(19) United States
(12) Patent Application Publication
(10) Pub. No.: US 2011/0045547 A1
(43) Pub. Date: Feb. 24, 2011

(54) ISOMERASES AND EPIMERASES AND METHODS OF USING

(76) Inventors: Mervyn L. de Souza, Fort Collins, CO (US); Wei Niu, San Diego, CA (US); Jose Laplaza, San Diego, CA (US); Christopher Solheid, Minneapolis, MN (US); Sherry R. Kollmann, Maple Grove, MN (US); Joshua M. Lundorff, Minneapolis, MN (US); Paula M. Hicks, Bend, OR (US); Sara C. McFarlan, St. Paul, MN (US); Fernando A. Sanchez-Riera, San Carlos, CA (US); David P. Weiner, Del Mar, CA (US); Ellen Burke, San Diego, CA (US); Peter Luginbuhl, San Diego, CA (US); Analia Bueno, San Diego, CA (US); Joslin Cuenca, San Diego, CA (US)

Correspondence Address: CARGILL, INCORPORATED, P.O. BOX 5624, MINNEAPOLIS, MN, 55440-5624, US

(21) Appl. No.: 12/828,714

(22) Filed: Jul. 1, 2010

Related U.S. Application Data
(63) Continuation of application No. PCT/US09/49599, filed on Jul. 2, 2009.
Application No. PCT/US09/49599 is a continuation of application No. PCT/US08/13968, filed on Dec. 22, 2008.
(60) Provisional application No. 61/018,845, filed on Jan. 3, 2008.

(51) Int. Cl. C12P 13/22; C12P 17/10

(52) U.S. Cl. 435/108; 435/121

(57) ABSTRACT

The invention provides for isomerase (e.g., racemase) and epimerase polypeptides and nucleic acids encoding such polypeptides. Also provided are methods of using such isomerase (e.g., racemase) and epimerase nucleic acids and polypeptides.

### CROSS REFERENCE TO RELATED APPLICATION

This application is a Continuation of, and claims benefit of priority to, International Application No. PCT/U.S.09/049,599 filed 2 Jul. 2009 and International Application No. PCT/U.S.08/013,968 filed 22 Dec. 2008, the latter of which claims benefit of priority under 35 U.S.C. §119(e) to U.S. Application No. 61/018,845 filed 3 Jan. 2008, all of which are hereby incorporated by reference in their entirety.

### INCORPORATION BY REFERENCE

A Sequence Listing is being electronically filed concurrently with the electronic filing of this application. The accompanying Sequence Listing, identified as 196080032002sequence.txt, is herein incorporated by reference.

Appendix I is being electronically filed concurrently with the electronic filing of this application. The accompanying Appendix I, identified as 196080032002appendix.pdf, is herein incorporated by reference.

LENGTHY TABLES
The patent application contains a lengthy table section. A copy of the table is available in electronic form from the USPTO web site (http://seqdata.uspto.gov/?pageRequest=docDetail&DocID=US20110045547A1). An electronic copy of the table will also be available from the USPTO upon request and payment of the fee set forth in 37 CFR 1.19(b)(3).

### TECHNICAL FIELD

This invention relates to nucleic acids and polypeptides, and more particularly to nucleic acids and polypeptides encoding isomerases (e.g., racemases) and epimerases as well as methods of using such isomerases and epimerases.

### BACKGROUND

Isomerases such as racemases as well as epimerases can catalyze the interconversion of substrate enantiomers. Isomerases and epimerases can catalyze the stereochemical inversion around an asymmetric carbon atom in a substrate having one or more centers of asymmetry.

### SUMMARY

This disclosure provides for a number of different isomerase (e.g., racemase) and epimerase polypeptides and the nucleic acids encoding such isomerase and epimerase polypeptides. This disclosure also provides for methods of using such isomerase and epimerase nucleic acids and polypeptides.

In one aspect, the invention provides methods of isomerizing a substrate. For example, one or more L-amino acids can be converted to the corresponding one or more D-amino acids (or, alternatively, one or more D-amino acids to the corresponding one or more L-amino acids). Such methods generally include combining one or more L-amino acids (or one or more D-amino acids) with a) one or more nucleic acid molecules chosen from the group consisting of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, and 497, wherein the one or more nucleic acid molecules encode polypeptides having isomerase or epimerase activity; b) a sequence variant of a), wherein the variant encodes a polypeptide having isomerase or epimerase activity; c) a fragment of a) or b), wherein the fragment encodes a polypeptide having isomerase or epimerase activity; d) one or more polypeptides chosen from the group consisting of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 442 D56N, 444, 446, 448, 450, 452, 454, 456, 458, 460, 462, 464, 466, 468, 470, 472, 474, 476, 478, 480, 482, 484, 486, 488, 490, 492, 494, 496, and 498, wherein the one or more polypeptides has isomerase or epimerase activity; e) a variant of d), wherein the variant has isomerase or epimerase activity; or f) a fragment of d) or e), wherein the fragment has isomerase or epimerase activity.

In one embodiment, the one or more nucleic acid molecules are chosen from the group consisting of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, and 497.

In one embodiment, the one or more polypeptides are chosen from the group consisting of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 386, 388, 390, 392, 394, 396, 398, 400, 402, 404, 406, 408, 410, 412, 414, 416, 418, 420, 422, 424, 426, 428, 430, 432, 434, 436, 438, 440, 442, 442 D56N, 444, 446, 448, 450, 452, 454, 456, 458, 460, 462, 464, 466, 468, 470, 472, 474, 476, 478, 480, 482, 484, 486, 488, 490, 492, 494, 496, and 498.

In certain embodiments, the nucleic acid molecule has the sequence shown in SEQ ID NO:411 and the polypeptide has the sequence shown in SEQ ID NO:412.

In certain embodiments, the variant is a nucleic acid molecule that has at least 98% (e.g., at least 99%) sequence identity to SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, and 497.

In certain embodiments, the variant is a polypeptide that has at least 98% (e.g., at least 99%) sequence identity to SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 442 D56N, 444, 446, 448, 450, 452, 454, 456, 458, 460, 462, 464, 466, 468, 470, 472, 474, 476, 478, 480, 482, 484, 486, 488, 490, 492, 494, 496, and 498.

In some embodiments, the variant is a nucleic acid that has at least 45% (e.g., at least 50%, at least 55%, at least 60%, at least 65%, at least 70%, at least 75%, at least 80%, at least 85%, at least 90%, at least 95%, or at least 99%) sequence identity to SEQ ID NO:411. In some embodiments, the variant is a polypeptide that has at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%) sequence identity to SEQ ID NO:412.

In some embodiments, the variant is a mutant. For example, a representative mutant has a mutation at the residue that aligns with residue 76 of A. caviae BAR. In some embodiments, the variant is a nucleic acid molecule that has been codon optimized. In one embodiment, the variant polypeptide is a chimeric polypeptide.

In certain embodiments, the nucleic acid molecule is contained within an expression vector and, for example, can be overexpressed. In certain embodiments, the isomerase or epimerase polypeptide lacks a signal sequence or a prepro domain. In some embodiments, the isomerase or epimerase polypeptide is immobilized on a solid support.

In certain embodiments, the polypeptide fragment is a PFAM domain. Representative polypeptide fragments that include a PFAM domain have the sequence shown in SEQ ID NO: 426, 440, or 462.

In one embodiment, the amino acid is tryptophan. In other embodiments, the amino acid is alanine. In some embodiments, the amino acid is a substituted amino acid.

In another aspect, the invention provides for methods of converting L-tryptophan to D-tryptophan (or, alternatively, D-tryptophan to L-tryptophan). Such methods typically include combining L-tryptophan (or D-tryptophan) with a) one or more nucleic acid molecules chosen from the group consisting of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, and 497, wherein the one or more nucleic acid molecules encode polypeptides having racemase activity; b) a variant of a), wherein the variant encodes a polypeptide having racemase activity; c) a fragment of a) or b), wherein the fragment encodes a polypeptide having racemase activity; d) one or more polypeptides chosen from the group consisting of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 386, 388, 390, 392, 394, 396, 398, 400, 402, 404, 406, 408, 410, 412, 414, 416, 418, 420, 422, 424, 426, 428, 430, 432, 434, 436, 438, 440, 442, 442 D56N, 444, 446, 448, 450, 452, 454, 456, 458, 460, 462, 464, 466, 468, 470, 472, 474, 476, 478, 480, 482, 484, 486, 488, 490, 492, 494, 496, and 498, wherein the one or more polypeptides has racemase activity; e) a variant of d), wherein the variant has racemase activity; or f) a fragment of d) or e), wherein the fragment has racemase activity.

Representative polypeptides include, without limitation, SEQ ID NOs:172, 178, 180, 182, 184, 140, 144, 188, 190, 112, 148, 156, 120, 162, and 108. Representative polypeptides also include, without limitation, SEQ ID NOs:136, 174, 138, 296, and 110. Representative polypeptides further include, without limitation, SEQ ID NOs:150, 192, 152, 118, 194, 154, 196, 158, 160, and 116. Representative polypeptides include, without limitation, SEQ ID NOs:248, 236, 246, 252, 250, 254, and 244. Representative polypeptides include, without limitation, SEQ ID NOs:274, 234, 220, 222, 226, 232, 240, 242, 258, 260, 264, 266, 286, 290, 170, 216, and 288. Representative polypeptides include, without limitation, SEQ ID NOs:208, 210, 228, 230, 270, 272, 278, 280, 282, 284, 292, 198, 212, 214, 114, and 218. Representative polypeptides include, without limitation, SEQ ID NOs:204 and 218.

In another aspect, the invention provides methods of converting L-tryptophan to D-tryptophan. Such methods generally include combining L-tryptophan with a) a nucleic acid molecule having the sequence shown in SEQ ID NO:411, wherein the nucleic acid molecule encodes a polypeptides having racemase activity; b) a variant of a), wherein the variant encodes a polypeptide having racemase activity; c) a fragment of a) or b), wherein the fragment encodes a polypeptide having racemase activity; d) one or more polypeptides chosen from the group consisting of SEQ ID NO:411, wherein the one or more polypeptides has racemase activity; e) a variant of d), wherein the variant has racemase activity; or f) a fragment of d) or e), wherein the fragment has racemase activity.

In one embodiment, the tryptophan is a substituted tryptophan. A representative substituted tryptophan is a chlorinated tryptophan (e.g., 6-chloro-D-tryptophan). In another embodiment, the substituted tryptophan is a halogenated tryptophan.

In yet another aspect, the invention provides methods of making monatin. Such methods generally include combining L-tryptophan with a) one or more nucleic acid molecules chosen from the group consisting of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, and 497, wherein the one or more nucleic acid molecules encode polypeptides having racemase activity; b) a variant of a), wherein the variant encodes a polypeptide having racemase activity; c) a fragment of a) or b), wherein the fragment encodes a polypeptide having racemase activity; d) one or more polypeptides chosen from the group consisting of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 386, 388, 390, 392, 394, 396, 398, 400, 402, 404, 406, 408, 410, 412, 414, 416, 418, 420, 422, 424, 426, 428, 430, 432, 434, 436, 438, 440, 442, 442 D56N, 444, 446, 448, 450, 452, 454, 456, 458, 460, 462, 464, 466, 468, 470, 472, 474, 476, 478, 480, 482, 484, 486, 488, 490, 492, 494, 496, and 498, wherein the one or more polypeptides has racemase activity; e) a variant of d), wherein the variant has racemase activity; or f) a fragment of d) or e), wherein the fragment has racemase activity.

In some embodiments, such methods further include adding one or more polypeptides having synthase/lyase (EC 4.1.3.- or EC 4.1.2.-) activity or a nucleic acid encoding such a polypeptide and/or one or more polypeptides having D-aminotransferase activity or a nucleic acid encoding such a polypeptide.

In certain embodiments, the monatin is predominantly R,R monatin. In one embodiment, the nucleic acid has the sequence shown in SEQ ID NO:411 and the polypeptide has the sequence shown in SEQ ID NO:412.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention, which may be in one or more embodiments of the invention, will be apparent from the drawings and detailed description, and from the claims.

### DETAILED DESCRIPTION

Disclosed herein are a number of different nucleic acid molecules encoding polypeptides having isomerase activity or epimerase activity. Isomerases such as racemases are provided herein that catalyze the racemization of a specific amino acid (e.g., tryptophan) or that catalyze the racemization of more than one amino acid (e.g., broad substrate racemases). In some embodiments, the nucleic acids or polypeptides disclosed herein can be used, for example, to convert L-tryptophan to D-tryptophan.

Isolated Nucleic Acid Molecules and Purified Polypeptides

The present invention is based, in part, on the identification of nucleic acid molecules encoding polypeptides having isomerase activity, herein referred to as “isomerase” or “racemase” nucleic acid molecules or polypeptides, where appropriate. The present invention also is based, in part, on the identification of nucleic acid molecules encoding polypeptides having epimerase activity, herein referred to as “epimerase” nucleic acid molecules or polypeptides, wherein appropriate.

Particular nucleic acid molecules described herein include the sequences shown in SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, and 497. As used herein, the term “nucleic acid molecule” can include DNA molecules and RNA molecules, analogs of DNA or RNA generated using nucleotide analogs. A nucleic acid molecule of the invention can be single-stranded or double-stranded, depending upon its intended use. Nucleic acid molecules of the invention include molecules that have at least, for example, 75% sequence identity (e.g., at least 80%, 85%, 90%, 95%, or 99% sequence identity) to any of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, and 497.and that have functional isomerase or epimerase activity.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It will be appreciated that a single sequence can align differently with other sequences and hence, can have different percent sequence identity values over each aligned region. It is noted that the percent identity value is usually rounded to the nearest integer. For example, 78.1%, 78.2%, 78.3%, and 78.4% are rounded down to 78%, while 78.5%, 78.6%, 78.7%, 78.8%, and 78.9% are rounded up to 79%. It is also noted that the length of the aligned region is always an integer.

The alignment of two or more sequences to determine percent sequence identity is performed using the algorithm described by Altschul et al. (1997, Nucleic Acids Res., 25:3389-3402) as incorporated into BLAST (basic local alignment search tool) programs, available at ncbi.nlm.nih.gov on the World Wide Web. BLAST searches can be performed to determine percent sequence identity between a DAT nucleic acid described herein and any other sequence or portion thereof aligned using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a sequence of the invention and another sequence, the default parameters of the respective programs are used.

Nucleic acid molecules of the invention, for example, those between about 10 and about 50 nucleotides in length, can be used, under standard amplification conditions, to amplify an isomerase or epimerase nucleic acid molecule. Amplification of an isomerase or epimerase nucleic acid can be for the purpose of detecting the presence or absence of an isomerase or epimerase nucleic acid molecule or for the purpose of obtaining (e.g., cloning) an isomerase or epimerase nucleic acid molecule. As used herein, standard amplification conditions refer to the basic components of an amplification reaction mix, and cycling conditions that include multiple cycles of denaturing the template nucleic acid, annealing the oligonucleotide primers to the template nucleic acid, and extension of the primers by the polymerase to produce an amplification product (see, for example, U.S. Pat. Nos. 4,683,195; 4,683,202; 4,800,159; and 4,965,188). The basic components of an amplification reaction mix generally include, for example, each of the four deoxynucleoside triphosphates, (e.g., dATP, dCTP, dTTP, and dGTP, or analogs thereof), oligonucleotide primers, template nucleic acid, and a polymerase enzyme. Template nucleic acid is typically denatured at a temperature of at least about 90° C., and extension from primers is typically performed at a temperature of at least about 72° C. In addition, variations to the original PCR methods (e.g., anchor PCR, RACE PCR, or ligation chain reaction (LCR)) have been developed and are known in the art. See, for example, Landegran et al., 1988, Science, 241:1077-1080; and Nakazawa et al., 1994, Proc. Natl. Acad. Sci. USA, 91:360-364).

The annealing temperature can be used to control the specificity of amplification. The temperature at which primers anneal to template nucleic acid must be below the Tm of each of the primers, but high enough to avoid non-specific annealing of primers to the template nucleic acid. The Tm is the temperature at which half of the DNA duplexes have separated into single strands, and can be predicted for an oligonucleotide primer using the formula provided in section 11.46 of Sambrook et al. (1989, Molecular Cloning: A Laboratory Manual, 2nd Ed., Cold Spring Harbor Laboratory Press, Cold Spring Harbor, N.Y.). Non-specific amplification products are detected as bands on a gel that are not the size expected for the correct amplification product.

Nucleic acid molecules of the invention, for example, those between about 10 and several hundred nucleotides in length (up to several thousand nucleotides in length), can be used, under standard hybridization conditions, to hybridize to an isomerase or epimerase nucleic acid molecule. Hybridization to an isomerase or epimerase nucleic acid molecule can be for the purpose of detecting or obtaining an isomerase or epimerase nucleic acid molecule. As used herein, standard hybridization conditions between nucleic acid molecules are discussed in detail in Sambrook et al. (1989, Molecular Cloning: A Laboratory Manual, 2^{nd} Ed., Cold Spring Harbor Laboratory Press, Cold Spring Harbor, N.Y.; Sections 7.37-7.57, 9.47-9.57, 11.7-11.8, and 11.45-11.57). For oligonucleotide probes less than about 100 nucleotides, Sambrook et al. discloses suitable Southern blot conditions in Sections 11.45-11.46. The Tm between a sequence that is less than 100 nucleotides in length and a second sequence can be calculated using the formula provided in Section 11.46. Sambrook et al. additionally discloses prehybridization and hybridization conditions for a Southern blot that uses oligonucleotide probes greater than about 100 nucleotides (see Sections 9.47-9.52). Hybridizations with an oligonucleotide greater than 100 nucleotides generally are performed 15-25° C. below the Tm. The Tm between a sequence greater than 100 nucleotides in length and a second sequence can be calculated using the formula provided in Sections 9.50-9.51 of Sambrook et al. Additionally, Sambrook et al. recommends the conditions indicated in Section 9.54 for washing a Southern blot that has been probed with an oligonucleotide greater than about 100 nucleotides.

The conditions under which membranes containing nucleic acids are prehybridized and hybridized, as well as the conditions under which membranes containing nucleic acids are washed to remove excess and non-specifically bound probe can play a significant role in the stringency of the hybridization. For example, hybridization and washing may be carried out under conditions of low stringency, moderate stringency or high stringency. Such conditions are described, for example, in Sambrook et al. section 11.45-11.46. The conditions used to achieve a particular level of stringency will vary, depending on the nature of the nucleic acids being hybridized. For example, the length, degree of complementarity, nucleotide sequence composition (e.g., G/C vs. A/T nucleotide content) and nucleic acid type (e.g., RNA v. DNA) of the hybridizing regions of the nucleic acids can be considered in selecting hybridization conditions. For example, washing conditions can be made more stringent by decreasing the salt concentration in the wash solutions and/or by increasing the temperature at which the washes are performed.

The amount of hybridization can be quantitated directly on a membrane or from an autoradiograph using, for example, a PhosphorImager or a Densitometer (Molecular Dynamics, Sunnyvale, Calif.). It is understood by those of skill in the art that interpreting the amount of hybridization can be affected by, for example, the specific activity of the labeled oligonucleotide probe, the number of probe-binding sites on the target nucleic acid, and the amount of exposure of an autoradiograph or other detection medium. It will be readily appreciated that, although any number of hybridization, washing and detection conditions can be used to examine hybridization of a probe nucleic acid molecule to immobilized target nucleic acids, it is more important to examine hybridization of a probe to target nucleic acids under identical hybridization, washing, and detection conditions. Preferably, the target nucleic acids are on the same membrane. In addition, it can be appreciated by those of skill in the art that appropriate positive and negative controls should be performed with every set of amplification or hybridization reactions to avoid uncertainties related to contamination and/or non-specific annealing of oligonucleotide primers or probes.

Oligonucleotide primers or probes specifically anneal or hybridize to one or more isomerase or epimerase nucleic acids. For amplification, a pair of oligonucleotide primers generally anneal to opposite strands of the template nucleic acid, and should be an appropriate distance from one another such that the polymerase can effectively polymerize across the region and such that the amplification product can be readily detected using, for example, electrophoresis. Oligonucleotide primers or probes can be designed using, for example, a computer program such as OLIGO (Molecular Biology Insights Inc., Cascade, Colo.) to assist in designing oligonucleotides. Typically, oligonucleotide primers are 10 to 30 or 40 or 50 nucleotides in length (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length), but can be longer or shorter if appropriate amplification conditions are used.

Non-limiting representative pairs of oligonucleotide primers that were used to amplify isomerase nucleic acid molecules are shown in Tables 16, 26, 35, 37 and 38 (e.g., SEQ ID NOs:503-515, 517-543, and 545-548). The sequences shown in SEQ ID NOs: 503-515, 517-543, and 545-548 are non-limiting examples of oligonucleotide primers that can be used to amplify isomerase nucleic acid molecules. Oligonucleotides in accordance with the invention can be obtained by restriction enzyme digestion of an isomerase or epimerase nucleic acid molecules or can be prepared by standard chemical synthesis and other known techniques.

As used herein, an “isolated” nucleic acid molecule is a nucleic acid molecule that is separated from other nucleic acid molecules that are usually associated with the isolated nucleic acid molecule. Thus, an “isolated” nucleic acid molecule includes, without limitation, a nucleic acid molecule that is free of sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule. In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA, or genomic library) or a portion of a gel (e.g., agarose, or polyacrylamine) containing restriction-digested genomic DNA is not to be considered an isolated nucleic acid.

Isolated nucleic acid molecules described herein having isomerase or epimerase activity can be obtained using techniques routine in the art, many of which are described in the Examples herein. For example, isolated nucleic acids within the scope of the invention can be obtained using any method including, without limitation, recombinant nucleic acid technology, the polymerase chain reaction (e.g., PCR, e.g., direct amplification or site-directed mutagenesis), and/or nucleic acid hybridization techniques (e.g., Southern blotting). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate an isomerase or epimerase nucleic acid molecule as described herein. Isolated nucleic acids in accordance with the invention also can be chemically synthesized, either as a single nucleic acid molecule or as a series of oligonucleotides.

Techniques for the manipulation of nucleic acids, such as, e.g., subcloning, labeling probes (e.g., random-primer labeling using Klenow polymerase, nick translation, amplification), sequencing, hybridization, amplification and the like are well described in the scientific and patent literature, see, e.g., Sambrook et al., Eds., 1989, Molecular Cloning: A Laboratory Manual (2^{nd} Ed.), Vols 1-3, Cold Spring Harbor Laboratory; Current Protocols in Molecular Biology, 1997, Ausubel, Ed. John Wiley & Sons, Inc., New York; Laboratory Techniques in Biochemistry and Molecular Biology: Hybridization with Nucleic Acid Probes, Part I. Theory and Nucleic Acid Preparation, Tijssen, Ed. Elsevier, N.Y. (1993).

Purified isomerase or epimerase polypeptides, as well as polypeptide fragments having isomerase or epimerase activity, are within the scope of the invention. Isomerase and epimerase polypeptides refer to polypeptides that catalyze the stereochemical inversion around an asymmetric carbon atom of a substrate. The predicted amino acid sequences of non-limiting exemplary isomerase and epimerase polypeptides are shown in SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, and 498. The term “purified” polypeptide as used herein refers to a polypeptide that has been separated from cellular components that naturally accompany it. Typically, a polypeptide is considered “purified” when it is at least partically free from the proteins and naturally occurring molecules with which it is naturally associated. The extent of enrichment or purity of an isomerase or epimerase polypeptide can be measured using any appropriate method, e.g., column chromatography, polyacrylamide gel electrophoresis, or HPLC analysis.

The invention also provides for isomerase and epimerase polypeptides that differ in sequence from any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, and 498. For example, the skilled artisan will appreciate that changes can be introduced into an isomerase or epimerase polypeptide (e.g., SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, and 498) or into an isomerase or epimerase nucleic acid molecule (e.g., SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, and 497), thereby leading to changes in the amino acid sequence of the encoded polypeptide. Isomerase and epimerase polypeptides that differ in sequence from SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, and 498 and that retain stereo-inverting activity readily can be identified by screening methods routinely used in the art.

For example, changes can be introduced into an isomerase or epimerase nucleic acid coding sequence that lead to conservative and/or non-conservative amino acid substitutions at one or more amino acid residues in the encoded isomerase or epimerase polypeptide. Polypeptides that differ in sequence from the amino acid sequences shown in SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, and 498 can be generated by standard techniques such as site-directed or PCR-mediated mutagenesis of a nucleic acid encoding such a polypeptide, or directed evolution. In addition, changes in the polypeptide sequence can be introduced randomly along all or part of the isomerase or epimerase polypeptide, such as by saturation mutagenesis of the corresponding nucleic acid molecule. Alternatively, changes can be introduced into a nucleic acid or polypeptide sequence by chemically synthesizing a nucleic acid molecule or polypeptide having such changes.

A “conservative amino acid substitution” is one in which one amino acid residue is replaced with a different amino acid residue having a similar side chain. Similarity between amino acid residues has been assessed in the art. For example, Dayhoff et al. (1978, in Atlas of Protein Sequence and Structure, 5 (Suppl. 3):345-352) provides frequency tables for amino acid substitutions that can be employed as a measure of amino acid similarity. Examples of conservative substitutions include, without limitation, replacement of an aliphatic amino acid with another aliphatic amino acid; replacement of a serine with a threonine or vice versa; replacement of an acidic residue with another acidic residue; replacement of a residue bearing an amide group with another residue bearing an amide group; exchange of a basic residue with another basic residue; or replacement of an aromatic residue with another aromatic residue. A non-conservative substitution is one in which an amino acid residue is replaced with an amino acid residue that does not have a similar side chain.

Changes in a nucleic acid can be introduced using one or more mutagens. Mutagens include, without limitation, ultraviolet light, gamma irradiation, or chemical mutagens (e.g., mitomycin, nitrous acid, photoactivated psoralens, sodium bisulfate, nitrous acid, hydroxylamine, hydrazine or formic acid). Other mutagens are analogues of nucleotide precursors, e.g., nitrosoguanidine, 5-bromouracil, 2-aminopurine, or acridine. Intercalating agents such as proflavine, acriflavine, quinacrine and the like can also be used.

Changes also can be introduced into an isomerase or epimerase nucleic acid and/or polypeptide by methods such as error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, gene reassembly (e.g., Gene Reassembly, see, e.g., U.S. Pat. No. 6,537,776), Gene Site Saturation Mutagenesis (GSSM), synthetic ligation reassembly (SLR), or a combination thereof. Changes also can be introduced into polypeptides by methods such as recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, or any combination thereof.

An isomerase or epimerase nucleic acid can be codon optimized if so desired. For example, a non-preferred or a less preferred codon can be identified and replaced with a preferred or neutrally used codon encoding the same amino acid as the replaced codon. A preferred codon is a codon over-represented in coding sequences in genes in the host cell and a non-preferred or less preferred codon is a codon under-represented in coding sequences in genes in the host cell, thereby modifying the nucleic acid to increase its expression in a host cell. An isomerase or epimerase nucleic acid can be optimized for particular codon usage from any host cell (e.g., any of the host cells described herein). See, for example, U.S. Pat. No. 5,795,737 for a representative description of codon optimization. In addition to codon optimization, a nucleic acid can undergo directed evolution. See, for example, U.S. Pat. No. 6,361,974.

Other changes also are within the scope of this disclosure. For example, one, two, three, four or more amino acids can be removed from the carboxy- and/or amino-terminal ends of an isomerase or epimerase polypeptide without significantly altering the biological activity. In addition, one or more amino acids can be changed to increase or decrease the pI of a polypeptide. In some embodiments, a residue can be changed to, for example, a glutamate. Also provided are chimeric isomerase or epimerase polypeptides. For example, a chimeric isomerase or epimerase polypeptide can include portions of different binding or catalytic domains. Methods of recombining different domains from different polypeptides and screening the resultant chimerics to find the best combination for a particular application or substrate are routine in the art.

One particular change in sequence exemplified herein involves the residue corresponding to residue 76 in A. caviae BAR. In one instance, the polypeptide sequence of SEQ ID NO:441 was aligned with A. caviae BAR and the residue in SEQ ID NO:441 that aligns with position 76 in A. caviae BAR was identified (residue 56) and changed from Asp to Asn (SEQ ID NO:441 with D56N). Those of skill in the art can readily identify the residue that corresponds to residue 76 from BAR A. caviae in any of the racemases disclosed herein and introduce a change into the polypeptide sequence at that particular residue.

By way of example, the invention provides for racemase polypeptides having the sequence shown in SEQ ID NOs:108, 110, 116, 244, 288 and 218 as well as racemase sequences that differ in sequence from SEQ ID NOs:108, 110, 116, 244, 288 and 218. For example, racemase polypeptides having the sequence shown in SEQ ID NOs: 172, 178, 180, 182, 184, 140, 144, 188, 190, 112, 148, 156, 120 and 162 each exhibit 96% or greater sequence identity to the racemase polypeptide having the sequence shown in SEQ ID NO: 108; while SEQ ID NOs:136, 174, 138 and 296 each exhibit 97% or greater sequence identity to SEQ ID NO:110. In addition, SEQ ID NOs:150, 192, 152, 118, 194, 154, 196, 158 and 160 each exhibit 97% or greater sequence identity to SEQ ID NO:116; and SEQ ID NOs:248, 236, 246, 252, 250 and 254 each exhibit 97% or greater sequence identity to SEQ ID NO:244. Also, SEQ ID NOs:274, 234, 220, 222, 226, 232, 240, 242, 258, 260, 264, 266, 286, 290, 170 and 216 each exhibit 97% or greater identity to SEQ ID NO:288; SEQ ID NOs:208, 210, 228, 230, 270, 272, 278, 280, 282, 284, 292, 198, 212, 214 and 114 each exhibit 97% or greater sequence identity to SEQ ID NO:218; and SEQ ID NO:204 exhibits 96% sequence identity to SEQ ID NO:218. These sequence identities between racemase polypeptides are exemplary and are not meant to be exhaustive of all possible sequence identities within or between the isomerase and epimerase nucleic acid and polypeptide sequences disclosed herein. Also as discussed herein, identifying and/or designing nucleic acid or polypeptide sequences that differ in sequence from, for example, one or more isomerase or epimerase sequences are well within the ordinary skill of those in the art.

In addition, the racemase polypeptide having the sequence shown in SEQ ID NO:412 is novel; the closest polypeptide sequence in the public databases exhibits only 23% sequence identity to SEQ ID NO:412. Therefore, polypeptides of the invention include polypeptides that have at least, for example, 25% sequence identity (e.g., at least 30%, 35%, 40%, 45%, 50%, 55%, 60%, 65%, 70%, 75%, 80%, 85%, 90%, 95%, or 99% sequence identity) to SEQ ID NO:412 or fragments thereof and that have functional racemase activity. The racemase polypeptide having the sequence shown in SEQ ID NO:412 is encoded by the nucleic acid having the sequence shown in SEQ ID NO:411. Similarly, SEQ ID NO:411 is a novel nucleic acid, for which the closest nucleic acid sequence in the public databases exhibits only 43% sequence identity to SEQ ID NO:411. Therefore, nucleic acid molecules of the invention include molecules that have at least, for example, 45% sequence identity (e.g., at least 50%, 55%, 60%, 65%, 70%, 75%, 80%, 85%, 90%, 95%, or 99% sequence identity) to SEQ ID NO:411 or fragments thereof and that encode a polypeptide that has functional racemase activity.

A fragment of an isomerase and epimerase nucleic acid or polypeptide refers to a portion of a full-length isomerase and epimerase nucleic acid or polypeptide. As used herein, “functional fragments” are those fragments of an isomerase or epimerase polypeptide that retain the respective enzymatic activity. “Functional fragments” also refer to fragments of an isomerase or epimerase nucleic acid that encode a polypeptide that retains the respective enzymatic activity. For example, functional fragments can be used in in vitro or in vivo reactions to catalyze transaminase or oxidation-reduction reactions, respectively. One example of a fragment, without limitation, is the PFAM domain from racemase polypeptides (PF01168 and PF00842; Finn et al., 2006, Nuc. Acids Res., Database Issue, 34:D247-D251). The PFAM domain is a fragment of a full-length racemase polypeptide that lacks about 30 to about 40 amino acids from the N-terminus of the polypeptide and also lacks about 10 to about 20 amino acids from the C-terminus of the polypeptide. The sequences of representative PFAM domains are shown in SEQ ID NOs:426, 440 and 462.

This disclosure provides for isomerase and epimerase polypeptides (and the nucleic acids encoding such polypeptides) lacking signal sequences (e.g., signal peptides) or prepro domains, and also provides for isomerases and epimerases that include signal sequences or prepro domains. The signal sequences or prepro domains can be isomerase or epimerase signal sequences or prepro domains, or such signal sequences or prepro domains can be obtained from non-isomerase, non-racemase and non-epimerase sequences. Such signal sequences or prepro domains can be operably linked to an isomerase or epimerase nucleic acid or polypeptide. A prepro domain can be located on the amino terminal or the carboxy terminal end of the polypeptide. Those in the art are familiar with SignalP, which can be used to identify signal peptides and cleavage sites. Representative signal sequences (also known as leader sequences) for racemase polypeptides include, without limitation, MHKKTLLATLIXGLLAGQAVA (SEQ ID NO:501), wherein X is F or L, and MPFRRTLLAASLALLITGQAPLYA (SEQ ID NO:502).

Isomerase or epimerase polypeptides can be obtained (e.g., purified) from natural sources (e.g., a biological sample) by known methods such as DEAE ion exchange, gel filtration, and hydroxyapatite chromatography. Natural sources include, but are not limited to, microorganisms such as bacteria and yeast. A purified isomerase or epimerase polypeptide also can be obtained, for example, by cloning and expressing an isomerase or epimerase nucleic acid (e.g., SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, and 497) and purifying the resultant polypeptide using, for example, any of the known expression systems including, but not limited to, glutathione S-transferase (GST), pGEX (Pharmacia Biotech Inc), pMAL (New England Biolabs, Beverly, Mass.) or pRITS (Pharmacia, Piscataway, N.J.)). In addition, a purified isomerase or epimerase polypeptide can be obtained by chemical synthesis using, for example, solid-phase synthesis techniques (see e.g., Roberge, 1995, Science, 269:202; Merrifield, 1997, Methods Enzymol., 289:3-13).

A purified isomerase or epimerase polypeptide or a fragment thereof can be used as an immunogen to generate polyclonal or monoclonal antibodies that have specific binding affinity for one or more isomerase or epimerase polypeptides. Such antibodies can be generated using standard techniques that are used routinely in the art. Full-length isomerase or epimerase polypeptides or, alternatively, antigenic fragments of isomerase or epimerase polypeptides can be used as immunogens. An antigenic fragment of an isomerase or epimerase polypeptide usually includes at least 8 (e.g., 10, 15, 20, or 30) amino acid residues of an isomerase or epimerase polypeptide (e.g., having the sequence shown in SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, and 498), and encompasses an epitope of an isomerase or epimerase polypeptide such that an antibody (e.g., polyclonal or monoclonal; chimeric or humanized) raised against the antigenic fragment has specific binding affinity for one or more isomerase or epimerase polypeptides.

Polypeptides can be detected and quantified by any method known in the art including, but not limited to, nuclear magnetic resonance (NMR), spectrophotometry, radiography (protein radiolabeling), electrophoresis, capillary electrophoresis, high performance liquid chromatography (HPLC), thin layer chromatography (TLC), hyperdiffusion chromatography, various immunological methods, e.g. immunoprecipitation, immunodiffusion, immuno-electrophoresis, radioimmunoassays (RIAs), enzyme-linked immunosorbent assays (ELISAs), immuno-fluorescent assays, gel electrophoresis (e.g., SDS-PAGE), staining with antibodies, fluorescent activated cell sorter (FACS), pyrolysis mass spectrometry, Fourier-Transform Infrared Spectrometry, Raman spectrometry, GC-MS, and LC-Electrospray and cap-LC-tandem-electrospray mass spectrometries, and the like. Novel bioactivities can also be screened using methods, or variations thereof, described in U.S. Pat. No. 6,057,103. Furthermore, one or more, or, all the polypeptides of a cell can be measured using a protein array.

Methods of Using Isomerase or Epimerase Nucleic Acids and Polypeptides

The isomerase or epimerase polypeptides or the isomerase or epimerase nucleic acids encoding such isomerase and epimerase polypeptides, respectively, can be used in the conversion of one or more L-amino acids to the corresponding D-amino acid(s). It is noted that the reactions described herein are not limited to any particular method, unless otherwise stated. In one embodiment, one or more of the racemase nucleic acids or polypeptides disclosed herein can be used to produce D-tryptophan (or another D-amino acid) from L-tryptophan (or another L-amino acid), or vice versa. The reactions disclosed herein can take place in vivo, in vitro, or a combination thereof.

Constructs containing isomerase or epimerase nucleic acid molecules are provided. Constructs, including expression vectors, suitable for expressing an isomerase or epimerase polypeptide are commercially available and/or readily produced by recombinant DNA technology methods routine in the art. Representative constructs or vectors include, without limitation, replicons (e.g., RNA replicons, bacteriophages), autonomous self-replicating circular or linear DNA or RNA, a viral vector (e.g., an adenovirus vector, a retroviral vector or an adeno-associated viral vector), a plasmid, a phage, a phagemid, a cosmid, a fosmid, a bacteriophage or an artificial chromosome. The cloning vehicle can comprise an artificial chromosome comprising a bacterial artificial chromosome (BAC), a bacteriophage P1-derived vector (PAC), a yeast artificial chromosome (YAC), or a mammalian artificial chromosome (MAC). Exemplary vectors include, without limitation, pBR322 (ATCC 37017), pKK223-3, pSVK3, pBPV, pMSG, and pSVL (Pharmacia Fine Chemicals, Uppsala, Sweden), GEM1 (Promega Biotech, Madison, Wis., USA) pQE70, pQE60, pQE-9 (Qiagen), pD10, psiX174 pBluescript II KS, pNH8A, pNH16a, pNH18A, pNH46A, pSV2CAT, pOG44, pXT1, pSG (Stratagene), ptrc99a, pKK223-3, pKK233-3, DR540, pRIT5 (Pharmacia), pKK232-8 and pCM7. See, also, U.S. Pat. No. 5,217,879 for a description of representative plasmids, viruses, and the like.

A vector or construct containing an isomerase or epimerase nucleic acid molecule can have elements necessary for expression operably linked to the isomerase or epimerase nucleic acid. Elements necessary for expression include nucleic acid sequences that direct and regulate expression of nucleic acid coding sequences. One example of an element necessary for expression is a promoter sequence. Promoter sequences are sequences that are capable of driving transcription of a coding sequence. A promoter sequence can be, for example, an isomerase or epimerase promoter sequence, or a non-isomerase or non-epimerase promoter sequence. Non-isomerase and non-epimerase promoters include, for example, bacterial promoters such as lacI, lacZ, T3, T7, gpt, lambda PR, lambdaPL and tip as well as eukaryotic promoters such as CMV immediate early, HSV thymidine kinase, early and late SV40, LTRs from retrovirus, and mouse metallothionein I. Promoters also can be, for example, constitutive, inducible, and/or tissue-specific. A representative constitutive promoter is the CaMV 35S; representative inducible promoters include, for example, arabinose, tetracycline-inducible and salicylic acid-responsive promoters.

Additional elements necessary for expression can include introns, enhancer sequences (e.g., an SV40 enhancer), response elements, or inducible elements that modulate expression of a nucleic acid. Elements necessary for expression also can include, for example, a ribosome binding site for translation initiation, splice donor and acceptor sites, and a transcription terminator. Elements necessary for expression can be of bacterial, yeast, insect, mammalian, or viral origin, and vectors or constructs can contain a combination of elements from different origins. Elements necessary for expression are described, for example, in Goeddel, 1990, Gene Expression Technology: Methods in Enzymology, 185, Academic Press, San Diego, Calif.

A vector or construct as described herein further can include sequences such as those encoding a selectable marker (e.g., genes encoding dihydrofolate reductase or genes conferring neomycin resistance for eukaryotic cells; genes conferring tetracycline or ampicillin resistance for E. coli; and the gene encoding TRP1 for S. cerevisiae), sequences that can be used in purification of an isomerase or epimerase polypeptide (e.g., 6×His tag), and one or more sequences involved in replication of the vector or construct (e.g., origins of replication). In addition, a vector or construct can contain, for example, one or two regions that have sequence homology for integrating the vector or construct. Vectors and constructs for genomic integration are well known in the art.

As used herein, operably linked means that a promoter and/or other regulatory element(s) are positioned in a vector or construct relative to an isomerase or epimerase nucleic acid in such a way as to direct or regulate expression of the isomerase or epimerase nucleic acid. Generally, promoter and other elements necessary for expression that are operably linked to a transcribed sequence are physically contiguous to the transcribed sequence, i.e., they are cis-acting. Some transcriptional regulatory sequences such as enhancers, however, need not be physically contiguous or located in close proximity to the coding sequences whose expression they enhance.

Also provided are host cells. Host cells generally contain a nucleic acid sequence of the invention, e.g., a sequence encoding an isomerase or an epimerase, or a vector or construct as described herein. The host cell may be any of the host cells familiar to those skilled in the art such as prokaryotic cells or eukaryotic cells including bacterial cells, fungal cells, yeast cells, mammalian cells, insect cells, or plant cells. Exemplary bacterial cells include any species within the genera Escherichia, Bacillus, Streptomyces, Salmonella, Pseudomonas and Staphylococcus, including, e.g., E. coli, L. lactis, B. subtilis, B. cereus, S. typhimurium, P. fluorescens. Exemplary fungal cells include any species of Aspergillus, and exemplary yeast cells include any species of Pichia, Saccharomyces, Schizosaccharomyces, or Schwanniomyces, including P. pastoris, S. cerevisiae, or S. pombe. Exemplary insect cells include any species of Spodoptera or Drosophila, including Drosophila S2 and Spodoptera Sf9. Exemplary animal cells include CHO, COS, Bowes melanoma, C127, 3T3, HeLa and BHK cell lines. See, for example, Gluzman, 1981, Cell, 23:175. The selection of an appropriate host is within the abilities of those skilled in the art.

Techniques for introducing nucleic acid into a wide variety of cells are well known and described in the technical and scientific literature. A vector or construct can be introduced into host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis et al., 1986, Basic Methods in Molecular Biology). Exemplary methods include CaPO₄ precipitation, liposome fusion, lipofection (e.g., LIPOFECTINT™), electroporation, viral infection, etc. The isomerase or epimerase nucleic acids may stably integrate into the genome of the host cell (for example, with retroviral introduction) or may exist either transiently or stably in the cytoplasm (i.e. through the use of traditional plasmids, utilizing standard regulatory sequences, selection markers, etc.).

The content of host cells usually is harvested by centrifugation, disrupted by physical or chemical means, and the resulting crude extract retained for further purification. Microbial cells employed for expression of proteins can be disrupted by any convenient method, including freeze-thaw cycling, sonication, mechanical disruption, or the use of cell lysing agents. Such methods are well known to those skilled in the art. The expressed polypeptide or fragment thereof can be recovered and purified from cell cultures by methods including, but not limited to, precipitation (e.g., ammonium sulfate or ethanol), acid extraction, chromatography (e.g., anion or cation exchange, phosphocellulose, hydrophobic interaction, affinity, hydroxylapatite and lectin). If desired, high performance liquid chromatography (HPLC) can be employed for final purification steps.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

An isomerase or epimerase polypeptide, a fragment, or a variant thereof can be assayed for activity by any number of methods. Methods of detecting or measuring the activity of an enzymatic polypeptide generally include combining a polypeptide, fragment or variant thereof with an appropriate substrate and determining whether the amount of substrate decreases and/or the amount of product increases. The substrates used to evaluate the activity of a number of racemases disclosed herein typically were one or more L-amino acids (e.g., L-tryptophan) and the products were the corresponding isomerized D-amino acid (e.g., D-tryptophan). In addition, racemases may show very little preference for or between substrate amino acids (e.g., broad activity racemases), while other racemases may exhibit a preference for one or more amino acids. In addition to L- or D-amino acid substrates, it is expected that polypeptides disclosed herein also will utilize substituted L- or D-amino acid substrates such as, without limitation, chlorinated tryptophan or 5-hydroxytryptophan. It is noted that D-isomers can be distinguished and/or separated from L-isomerase using methods known in the art including, but not limited to, chiral chromatography, simulated moving bed (SMB) continuous chromatography, chiral ausiliaries, and/or enzymatic resolution.

One method for evaluating racemase activity is described in Schonfeld & Bornscheuer (2004, Anal. Chem., 76(4):1184-8), which describes a polarimetric assay that indentifies alpha-amino acid racemase activity using a glutamate racemase from Lactobacillus fermentii expressed in E. coli, and measuring the time-dependent change of the optical rotation using L-glutamate as the substrate. In addition, methods of evaluating candidate polypeptides for racemase activity are described in Part A and Part B of the Example section herein. For the purposes of determining whether or not a polypeptide falls within the scope of the invention, the methods described in Part B of the Example section are employed.

Typically, an isomerase or epimerase polypeptide exhibits activity in the range of between about 0.05 to 20 units (e.g., about 0.10, 0.20, 0.30, 0.40, 0.50, 0.60, 0.70, 0.80, 0.90, 1.0, 1.5, 2.0, 2.5, 3.0, 3.5, 4.0, 4.5, 5.0, 5.5, 6.0, 6.5, 7, 8, 9, 10, 11, 12, 13, 14, 15, 16, 17, 18, 19 or 19.5 or more units). As used herein, a unit equals one μmol of product released per minute per mg of enzyme. In one embodiment, one unit of activity for a racemase polypeptide is one μmol of an isomer with inverted configuration (from the starting isomer) produced per minute per mg of enzyme (formed from the respective alpha-amino acid or amine). In an alternative embodiment, one unit of activity for an amino acid racemase is one μmol of R-amino acid produced per minute per mg of enzyme formed from the corresponding S-amino acid. In an alternative embodiment, one unit of activity for an amino acid racemase is one μmol of S-amino acid produced per minute per mg of enzyme formed from the corresponding R-amino acid.

The conversion of L-tryptophan to D-tryptophan using one or more of the isomerase or epimerase nucleic acids or polypeptides disclosed herein can be performed in vitro or in vivo, in solution or in a host cell, in series or in parallel. When one or more reactions are performed in vitro, the desired ingredients for the reaction(s) can be combined by admixture in an aqueous reaction medium or solution and maintained for a period of time sufficient for the desired products) to be produced. Alternatively, one or more isomerase or epimerase polypeptides used in the one or more of the reactions described herein can be immobilized onto a solid support. Examples of solid supports include those that contain epoxy, aldehyde, chelators, or primary amine groups. Specific examples of suitable solid supports include, but are not limited to, Eupergit® C (Rohm and Haas Company, Philadelphia, Pa.) resin beads and SEPABEADS® EC-EP (Resindion).

To generate D-tryptophan from L-tryptophan in vivo, an isomerase or epimerase nucleic acid (e.g., an expression vector) can be introduced into any of the host cells described herein. Depending upon the host cell, many or all of the co-factors (e.g., a metal ion, a coenzyme, a pyridoxal-phosphate, or a phosphopantetheine) and/or substrates necessary for the conversion reactions to take place can be provided in the culture medium. After allowing the in vitro or in vivo reaction to proceed, the efficiency of the conversion can be evaluated by determining whether the amount of substrate has decreased or the amount of product has increased.

In some embodiments, the activity of one or more of the isomerase or epimerase polypeptides disclosed herein can be improved or optimized using any number of strategies known to those of skill in the art. For example, the in vivo or in vitro conditions under which one or more reactions are performed such as pH or temperature can be adjusted to improve or optimize the activity of a polypeptide. In addition, the activity of a polypeptide can be improved or optimized by re-cloning the isomerase or epimerase nucleic acid into a different vector or construct and/or by using a different host cell. For example, a host cell can be used that has been genetically engineered or selected to exhibit increased uptake or production of tryptophan (see, for example, U.S. Pat. No. 5,728,555). Further, the activity of an isomerase or epimerase polypeptide can be improved or optimized by ensuring or assisting in the proper folding of the polypeptide (e.g., by using chaperone polypeptides) or in the proper post-translational modifications such as, but not limited to, acetylation, acylation, ADP-ribosylation, amidation, glycosylation, hydroxylation, iodination, methylation, myristolyation, oxidation, pegylation, phosphorylation, prenylation, selenoylation, sulfation, disulfide bond formation, and demethylation as well as covalent attachment of molecules such as flavin, a heme moiety, a nucleotide or nucleotide derivative, a lipid or lipid derivative, and/or a phosphytidylinositol. In addition, the solubility of a polypeptide can be increased using any number of methods known in the art such as, but not limited to, low temperature expression or periplasmic expression.

A number of polypeptides were identified herein that exhibit racemase activity. Specifically, SEQ ID NOs:412, 400, 406, 410, 408, 416, 418, 424, 440, 442, 444, 446, 454, 442, 474, 476, 322, 336, 338 and 442 exhibit isomerase activity. As disclosed herein, the sequence shown in SEQ ID NO:412 represents a very unique racemase, as the most homologous sequence in the public databases has only 30% sequence identity to SEQ ID NO:412. Additionally, despite the fact that SEQ ID NO:412 exhibited low solubility in its native form, SEQ ID NO:412 still exhibits very effective racemase activity. SEQ ID NO:322 also is unique as the encoded polypeptide is only 232 amino acids, making SEQ ID NO:322 the smallest active polypeptide identified.

Use of Isomerase or Epimerase Nucleic Acids or Polypeptides in the Production of Monatin and Monatin Derivatives

Monatin is a high-intensity sweetener having the chemical formula:

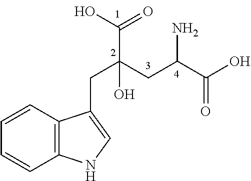

Monatin includes two chiral centers leading to four potential stereoisomeric configurations. The R,R configuration (the “R,R stereoisomer” or “R,R monatin”); the S,S configuration (the “S,S stereoisomer” or “S,S monatin”); the R,S configuration (the “R,S stereoisomer” or “R,S monatin”); and the S,R configuration (the “S,R stereoisomer” or “S,R monatin”). As used herein, unless stated otherwise, the term “monatin” is used to refer to compositions including all four stereoisomers of monatin, compositions including any combination of monatin stereoisomers, (e.g., a composition including only the R,R and S,S, stereoisomers of monatin), as well as a single isomeric form (or any of the salts thereof). Due to various numbering systems for monatin, monatin is known by a number of alternative chemical names, including: 2-hydroxy-2-(indol-3-ylmethyl)-4-aminoglutaric acid; 4-amino-2-hydroxy-2-(1H-indol-3-ylmethyl)-pentanedioic acid; 4-hydroxy-4-(3-indolylmethyl)glutamic acid; and, 3-(1-amino-1,3-dicarboxy-3-hydroxy-but-4-yl)indole.

Methods of producing various stereoisomers of monatin (e.g., R,R monatin) are disclosed in, for example, WO 07/133,183 and WO 07/103,389. One or more of the racemase polypeptides disclosed herein, in the presence of L-tryptophan, can be used in methods known to those of skill in the art to make a monatin composition. As disclosed in both WO 07/133,183 and WO 07/103,389, the conversion of indole-3-pyruvate (or derivatives thereof; see, for example, WO 07/103,389) to 2-hydroxy2-(indol-3-ylmethyl)-4-keto glutaric acid (“monatin precursor” or “MP”) dictates the first chiral center of monatin, while the conversion of MP to monatin dictates the second chiral center. Therefore, the racemases disclosed herein, with or without another polypeptide (e.g., an R-specific aldolase as disclosed in WO 07/103,389 and/or a D-aminotransferase as disclosed in co-pending U.S. Application No. 61/018,814 can be used to generate a desired percentage or a minimum or maximum desired percentage of one or more particular monatin stereoisomers (e.g., R,R monatin) relative to other monatin stereoisomers (e.g., S,R monatin). In some embodiments, amino acid racemases that do not isomerize significant amounts of monatin are used rather than racemases that isomerize monatin as a method of maintaining the desired percentage of one or more particular monatin stereoisomers.

Monatin that is produced utilizing one or more of the racemase polypeptides disclosed herein can be at least about 0.5-30% R,R-monatin by weight of the total monatin produced. In other embodiments, the monatin produced using one or more of the polypeptides disclosed herein, is greater than 30% R,R-monatin, by weight of the total monatin produced; for example, the R,R-monatin is 40%, 50%, 60%, 65%, 70%, 75%, 80%, 85%, 90%, 95% or 99% of the total monatin produced. Alternatively, various amounts of two or more preparations of monatin can be combined so as to result in a preparation that is a desired percentage of R,R-monatin.

Monatin produced using one or more of the racemase polypeptides disclosed herein can be, for example, a derivative. “Monatin derivatives” have the following structure:

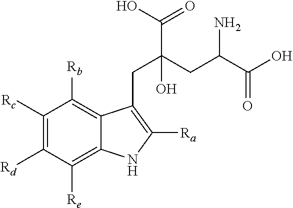

wherein, Rₐ, R_{b}, R_{c}, R_{d}, and Rₑ each independently represent any substituent selected from a hydrogen atom, a hydroxyl group, a C₁-C₃ alkyl group, a C₁-C₃ alkoxy group, an amino group, or a halogen atom, such as an iodine atom, bromine atom, chlorine atom, or fluorine atom. However, Rₐ, R_{b}, R_{c}, R_{d}, and Rₑ cannot simultaneously all be hydrogen. Alternatively, R_{b} and R_{c}, and/or R_{d} and Rₑ may together form a C₁-C₄ alkylene group, respectively. “Substituted monatin” refers to, for example, halogenated or chlorinated monatin or monatin derivatives. See, for example, U.S. Publication No. 2005/0118317.

Monatin derivatives also can be used as sweeteners. For example, chlorinated D-tryptophan, particularly 6-chloro-D-tryptophan, which has structural similarities to R,R monatin, has been identified as a non-nutritive sweetener. Similarly, halogenated and hydroxy-substituted forms of monatin have been found to be sweet. See, for example, U.S. Publication No. 2005/0118317. Substituted indoles have been shown in the literature to be suitable substrates and have yielded substituted tryptophans. See, for example, Fukuda et al., 1971, Appl. Environ. Microbiol, 21:841-43. The halogen does not appear to sterically hinder the catalytic mechanism or the enantiospecificity of the enzyme. Therefore, halogens and hydroxyl groups should be substitutable for hydrogen, particularly at positions 1-4 of the benzene ring in the indole of tryptophan, without interfering in subsequent conversions to D- or L-tryptophan, indole-3-pyruvate, MP, or monatin.

In accordance with the present invention, there may be employed conventional molecular biology, microbiology, biochemical, and chemical techniques within the skill of the art. Such techniques are explained fully in the literature. The invention will be further described in the following examples, which do not limit the scope of the invention described in the claims.

### EXAMPLES

The Examples in Part A describe the methodologies used for characterization of the candidate isomerase and epimerase nucleic acids and the encoded polypeptides. Additional characterization of a subset of the isomerase and epimerase nucleic acids and polypeptides, particularly the nucleic acids encoding amino acid racemases, is described in Part B.

### Part A

### Example 1

### Effect of Leader Sequence on Racemase

Many of the racemases that were discovered had native signal/leader sequences. The signal sequences and corresponding cleavage sites were identified by SignalP 3.0 (at cbs.dtu.dk/services/SignalP/ on the World Wide Web). It was observed that clones containing racemases with leader sequences tended to be more difficult to grow. The clones grew well with fresh transformations, however they did not grow well when they were subcultured or inoculated from glycerol stocks. Samples were grown (or, at least, attempted) a minimum of two times.

The table below indicates several clones that contained their native signal sequences. These samples were in the PCR4-TOPO vector/Top10 host (Invitrogen, Carlsbad, Calif.). Growth conditions were over-night in LB/kanamycin 50 μg/mL, 37° C. All of these samples were difficult to grow. Nineteen of the clones have the following leader sequence: MHKKTLLATLIFGLLAGQAVA (SEQ ID NO:499). Seventeen of the clones have a leader sequence that differs by one amino acid: MHKKTLLATLILGLLAGQAVA (SEQ ID NO:500). Therefore, the consensus sequence for racemase leader sequences is MHKKTLLATLIXGLLAGQAVA (SEQ ID NO:501) where X is F or L. Table 1 shows the leader sequences that were identified.

TABLE 1
Leadered racemase clones

| From clone | | | | |
|---|---|---|---|---|
| having SEQ | | | | SEQ ID |
| ID NO | Source | Signalp Cleavage Site | Predicted Signal Sequence | NO: |
| 41, 42 | Unknown | Probability: 0.999 AA1: | MPFCRTLLAVSLGLLITGQAPLYA | 550 |
| | | 24 AA2: 25 | | |
| 43, 44 | Unknown | Probability: 0.999 AA1: | MPFCRTLLAVSLGLLITGQAPLYA | 550 |
| | | 24 AA2: 25 | | |
| 45, 46 | Unknown | Probability: 1.000 AA1: | MPFSRTLLAVSLGLLITGQAPLYA | 551 |
| | | 24 AA2: 25 | | |
| 47, 48 | Unknown | Probability: 1.000 AA1: | MPFSRTLLAVSLGLLITGQAPLYA | 551 |
| | | 24 AA2: 25 | | |
| 49, 50 | Unknown | Probability: 1.000 AA1: | MPFCRTLLAASLALLITGQAPLYA | 552 |
| | | 24 AA2: 25 | | |
| 51, 52 | Unknown | Probability: 1.000 AA1: | MPFRRTLLALSLGLVLWQGQVHA | 553 |
| | | 23 AA2: 24 | | |
| 53, 54 | Unknown | Probability: 1.000 AA1: | MPFCRTLLALSLGLVLWQGQAHA | 554 |
| | | 23 AA2: 24 | | |
| 55, 56 | Unknown | Probability: 1.000 AA1: | MPFCRTLLALSLGLVLWQGQVHA | 555 |
| | | 23 AA2: 24 | | |
| 57, 58 | Unknown | Probability: 1.000 AA1: | MPFSRTLLAASLALLITGQAPLYA | 556 |
| | | 24 AA2: 25 | | |
| 59, 60 | Unknown | Probability: 1.000 AA1: | MPFCRTLLAASLALLITGQAPLYA | 552 |
| | | 24 AA2: 25 | | |
| 61, 62 | Unknown | Probability: 0.999 AA1: | MFTMIFMKKKFCLLFATIILFITCL | 557 |
| | | 33 AA2: 34 | CFLLKSVS | |
| 63, 64 | Unknown | Probability: 1.000 AA1: | MPFRRTLLAASLALLITGLAPLYA | 558 |
| | | 24 AA2: 25 | | |
| 65, 66 | Unknown | Probability: 1.000 AA1: | MPFPRTLLAASLALLITGQAPLYA | 559 |
| | | 24 AA2: 25 | | |
| 67, 68 | Unknown | Probability: 1.000 AA1: | MPFRRTLLAASLALLVTAQAPLYA | 560 |
| | | 24 AA2: 25 | | |
| 69, 70 | Unknown | Probability: 1.000 AA1: | MPFCRTLLAASLALLITGQAPLYA | 552 |
| | | 24 AA2: 25 | | |
| 71, 72 | Unknown | Probability: 1.000 AA1: | MPFRRTLLAASLALLITGQAPLYA | 502 |
| | | 24 AA2: 25 | | |
| 73, 74 | Unknown | Probability: 1.000 AA1: | MPFCRTLLAASLALLITGQAPLYA | 552 |
| | | 24 AA2: 25 | | |
| 75, 76 | Unknown | Probability: 1.000 AA1: | MPFPRTLLAASLALLITGQAPLYA | 559 |
| | | 24 AA2: 25 | | |
| 77, 78 | Unknown | Probability: 1.000 AA1: | MPFRRTLLAASLALLITGQAPLYA | 502 |
| | | 24 AA2: 25 | | |
| 79, 80 | Unknown | Probability: 1.000 AA1: | MPFCRTLLAASLALLITGQAPLYA | 552 |
| | | 24 AA2: 25 | | |
| 81, 82 | Unknown | Probability: 1.000 AA1: | MPFCRTLLAASLALLITGQAPLYA | 552 |
| | | 24 AA2: 25 | | |
| 83, 84 | Unknown | Probability: 1.000 AA1: | MPFCRTLLAASLALLITGQAPLYA | 552 |
| | | 24 AA2: 25 | | |
| 85, 86 | Unknown | Probability: 1.000 AA1: | MPFSRTLLAASLALLITGQAPLFA | 561 |
| | | 24 AA2: 25 | | |
| 87, 88 | Unknown | Probability: 1.000 AA1: | MPFCRTLLAASLALLITGQAPLYA | 552 |
| | | 24 AA2: 25 | | |
| 89, 90 | Unknown | Probability: 1.000 AA1: | MPFRRTLLAASLALLITGQAPLFA | 562 |
| | | 24 AA2: 25 | | |
| 91, 92 | Unknown | Probability: 1.000 AA1: | MPFCRTLLAASLALLITGQAPLYA | 552 |
| | | 24 AA2: 25 | | |
| 93, 94 | Unknown | Probability: 1.000 AA1: | MPFRRTLLAASLALLITGQAPLYA | 502 |
| | | 24 AA2: 25 | | |
| 95, 96 | Unknown | Probability: 1.000 AA1: | MPFPRTLLAASLALLITGQAPLYA | 559 |
| | | 24 AA2: 25 | | |
| 97, 98 | Unknown | Probability: 1.000 AA1: | MPFRRTLLAASLALLITGQAPLYA | 502 |
| | | 24 AA2: 25 | | |
| 99, 100 | Unknown | Probability: 1.000 AA1: | MPFCRTLLAASLALLITGQAPLYA | 552 |
| | | 24 AA2: 25 | | |
| 103, 104 | Unknown | Probability: 0.826 AA1: | MKNNKCIAILGGMGPQASS | 563 |
| | | 19 AA2: 20 | | |
| 107, 108 | Unknown | Probability: 1.000 AA1: | MHKKTLLATLILGLLAGQAVA | 500 |
| | | 21 AA2: 22 | | |
| 109, 110 | Unknown | Probability: 1.000 AA1: | MHKKTLLATLIFGLLAGQAVA | 499 |
| | | 21 AA2: 22 | | |
| 111, 112 | Unknown | Probability: 1.000 AA1: | MHKKTLLATLILGLLAGQAVA | 500 |
| | | 21 AA2: 22 | | |
| 113, 114 | Unknown | Probability: 1.000 AA1: | MHKKTLLATLVFGLLAGQAVA | 564 |
| | | 21 AA2: 22 | | |
| 115, 116 | Unknown | Probability: 1.000 AA1: | MHKKTLLATLVFGLLAGQAVA | 564 |
| | | 21 AA2: 22 | | |
| 117, 118 | Unknown | Probability: 1.000 AA1: | MHKKTLLATLVFGLLAGQAVA | 564 |
| | | 21 AA2: 22 | | |
| 119, 120 | Unknown | Probability: 1.000 AA1: | MHKKTLLATLVFGLLAGQAVA | 564 |
| | | 21 AA2: 22 | | |
| 133, 134 | Unknown | Probability: 1.000 AA1: | MHKKTLLATLILGLLAGQAVA | 500 |
| | | 21 AA2: 22 | | |
| 135, 136 | Unknown | Probability: 1.000 AA1: | MHKKTLLATLIFGLLAGQAVA | 499 |
| | | 21 AA2: 22 | | |
| 137, 138 | Unknown | Probability: 1.000 AA1: | MHKKTLLATLIFGLLAGQAVA | 499 |
| | | 21 AA2: 22 | | |
| 139, 140 | Unknown | Probability: 1.000 AA1: | MHKKTLLATLILGLLAGQAVA | 500 |
| | | 21 AA2: 22 | | |
| 141, 142 | Unknown | Probability: 1.000 AA1: | MHKKTLLATLIFGLLAGQAVA | 499 |
| | | 21 AA2: 22 | | |
| 143, 144 | Unknown | Probability: 1.000 AA1: | MHKKTLLATLILGLLAGQAVA | 500 |
| | | 21 AA2: 22 | | |
| 145, 146 | Unknown | Probability: 1.000 AA1: | MHKKTLLATLILGLLAGQAVA | 500 |
| | | 21 AA2: 22 | | |
| 147, 148 | Unknown | Probability: 1.000 AA1: | MHKKTLLATLILGLLAGQAVA | 500 |
| | | 21 AA2: 22 | | |
| 149, 150 | Unknown | Probability: 1.000 AA1: | MHKKTLLATLVFGLLAGQAVA | 564 |
| | | 21 AA2: 22 | | |
| 151, 152 | Unknown | Probability: 1.000 AA1: | MHKKTLLATLVFGLLAGQAVA | 564 |
| | | 21 AA2: 22 | | |
| 153, 154 | Unknown | Probability: 1.000 AA1: | MHKKTLLATLILGLLAGQAVA | 500 |
| | | 21 AA2: 22 | | |
| 155, 156 | Unknown | Probability: 1.000 AA1: | MHKKTLLATLILGLLAGQAVA | 500 |
| | | 21 AA2: 22 | | |
| 157, 158 | Unknown | Probability: 1.000 AA1: | MHKKTLLATLVFGLLAGQAVA | 564 |
| | | 21 AA2: 22 | | |
| 159, 160 | Unknown | Probability: 1.000 AA1: | MHKKTLLATLVFGLLAGQAVA | 564 |
| | | 21 AA2: 22 | | |
| 161, 162 | Unknown | Probability: 1.000 AA1: | MHKKTLLATLILGLLAGQAVA | 500 |
| | | 21 AA2: 22 | | |
| 169, 170 | Unknown | Probability: 1.000 AA1: | MHKKTLLATLILGLLAGQAVA | 500 |
| | | 21 AA2: 22 | | |
| 171, 172 | Unknown | Probability: 1.000 AA1: | MHKKTLLATLILGLLAGQAVA | 500 |
| | | 21 AA2: 22 | | |
| 173, 174 | Unknown | Probability: 1.000 AA1: | MHKKTLLATLIFGLLAGQAVA | 499 |
| | | 21 AA2: 22 | | |
| 175, 176 | Unknown | Probability: 1.000 AA1: | MHKKTLLATLVFGLLAGQAVA | 564 |
| | | 21 AA2: 22 | | |
| 177, 178 | Unknown | Probability: 1.000 AA1: | MHKKTLLATLILGLLAGQAVA | 500 |
| | | 21 AA2: 22 | | |
| 179, 180 | Unknown | Probability: 1.000 AA1: | MHKKTLLATLILGLLAGQAVA | 500 |
| | | 21 AA2: 22 | | |
| 181, 182 | Unknown | Probability: 1.000 AA1: | MHKKTLLATLILGLLAGQAVA | 500 |
| | | 21 AA2: 22 | | |
| 183, 184 | Unknown | Probability: 1.000 AA1: | MHKKTLLATLILGLLAGQAVA | 500 |
| | | 21 AA2: 22 | | |
| 185, 186 | Unknown | Probability: 1.000 AA1: | MHKKTLLATLIFGLLAGQAVA | 499 |
| | | 21 AA2: 22 | | |
| 187, 188 | Unknown | Probability: 1.000 AA1: | MHKKTLLATLILGLLAGQAVA | 500 |
| | | 21 AA2: 22 | | |
| 189, 190 | Unknown | Probability: 1.000 AA1: | MHKKTLLATLILGLLAGQAVA | 500 |
| | | 21 AA2: 22 | | |
| 191, 192 | Unknown | Probability: 1.000 AA1: | MHKKTLLATLVFGLLAGQAVA | 564 |
| | | 21 AA2: 22 | | |
| 193, 194 | Unknown | Probability: 1.000 AA1: | MHKKTLLATLVFGLLAGQAVA | 564 |
| | | 21 AA2: 22 | | |
| 195, 196 | Unknown | Probability: 1.000 AA1: | MHKKTLLATLVFGLLAGQAVA | 564 |
| | | 21 AA2: 22 | | |
| 197, 198 | Unknown | Probability: 1.000 AA1: | MHKKTLLATLVFGLLAGQAVA | 564 |
| | | 21 AA2: 22 | | |
| 199, 200 | Unknown | Probability: 1.000 AA1: | MHKKTLLATLIFGLLAGQAVA | 499 |
| | | 21 AA2: 22 | | |
| 201, 202 | Unknown | Probability: 1.000 AA1: | MHKKTLLATLIFGLLAGQAVA | 499 |
| | | 21 AA2: 22 | | |
| 203, 204 | Unknown | Probability: 1.000 AA1: | MHKKTLLATLIFGLLAGQAVA | 499 |
| | | 21 AA2: 22 | | |
| 205, 206 | Unknown | Probability: 1.000 AA1: | MHKKTLLATLIFGLLAGQAVA | 499 |
| | | 21 AA2: 22 | | |
| 207, 208 | Unknown | Probability: 1.000 AA1: | MHKKTLLATLVFGLLAGQAVA | 564 |
| | | 21 AA2: 22 | | |
| 209, 210 | Unknown | Probability: 1.000 AA1: | MHKKTLLATLVFGLLAGQAVA | 564 |
| | | 21 AA2: 22 | | |
| 211, 212 | Unknown | Probability: 1.000 AA1: | MHKKTLLATLVFGLLAGQAVA | 564 |
| | | 21 AA2: 22 | | |
| 213, 214 | Unknown | Probability: 1.000 AA1: | MHKKTLLATLVFGLLAGQAVA | 564 |
| | | 21 AA2: 22 | | |
| 215, 216 | Unknown | Probability: 1.000 AA1: | MHKKTLLATLILGLLAGQAVA | 500 |
| | | 21 AA2: 22 | | |
| 217, 218 | Unknown | Probability: 1.000 AA1: | MHKKTLLATLVFGLLAGQAVA | 564 |
| | | 21 AA2: 22 | | |
| 219, 220 | Unknown | Probability: 1.000 AA1: | MHKKTLLATLILGLLAGQAVA | 500 |
| | | 21 AA2: 22 | | |
| 221, 222 | Unknown | Probability: 1.000 AA1: | MHKKTLLATLILGLLAGQAVA | 500 |
| | | 21 AA2: 22 | | |
| 223, 224 | Unknown | Probability: 1.000 AA1: | MHKKTLLATLILGLLAGQAVA | 500 |
| | | 21 AA2: 22 | | |
| 225, 226 | Unknown | Probability: 1.000 AA1: | MHKKTLLATLILGLLAGQAVA | 500 |
| | | 21 AA2: 22 | | |
| 227, 228 | Unknown | Probability: 1.000 AA1: | MHKKTLLATLVFGLLAGQAVA | 564 |
| | | 21 AA2: 22 | | |
| 229, 230 | Unknown | Probability: 1.000 AA1: | MHKKTLLATLVFGLLAGQAVA | 564 |
| | | 21 AA2: 22 | | |
| 231, 232 | Unknown | Probability: 1.000 AA1: | MHKKTLLATLILGLLAGQAVA | 500 |
| | | 21 AA2: 22 | | |
| 233, 234 | Unknown | Probability: 1.000 AA1: | MHKKTLLATLILGLLAGQAVA | 500 |
| | | 21 AA2: 22 | | |
| 235, 236 | Unknown | Probability: 1.000 AA1: | MHKKTLLATLIFGLLAGQAVA | 499 |
| | | 21 AA2: 22 | | |
| 237, 238 | Unknown | Probability: 1.000 AA1: | MHKKTLLATLILGLLAGQAVA | 500 |
| | | 21 AA2: 22 | | |
| 239, 240 | Unknown | Probability: 1.000 AA1: | MHKKTLLATLILGLLAGQAVA | 500 |
| | | 21 AA2: 22 | | |
| 241, 242 | Unknown | Probability: 1.000 AA1: | MHKKTLLATLILGLLAGQAVA | 500 |
| | | 21 AA2: 22 | | |
| 243, 244 | Unknown | Probability: 1.000 AA1: | MHKKTLLATLIFGLLAGQAVA | 499 |
| | | 21 AA2: 22 | | |
| 245, 246 | Unknown | Probability: 1.000 AA1: | MHKKTLLATLIFGLLAGQAVA | 499 |
| | | 21 AA2: 22 | | |
| 247, 248 | Unknown | Probability: 1.000 AA1: | MHKKTLLATLIFGLLAGQAVA | 499 |
| | | 21 AA2: 22 | | |
| 249, 250 | Unknown | Probability: 1.000 AA1: | MHKKTLLATLIFGLLAGQAVA | 499 |
| | | 21 AA2: 22 | | |
| 251, 252 | Unknown | Probability: 1.000 AA1: | MHKKTLLATLIFGLLAGQAVA | 499 |
| | | 21 AA2: 22 | | |
| 253, 254 | Unknown | Probability: 1.000 AA1: | MHKKTLLATLIFGLLAGQAVA | 499 |
| | | 21 AA2: 22 | | |
| 257, 258 | Unknown | Probability: 1.000 AA1: | MHKKTLLATLILGLLAGQAVA | 500 |
| | | 21 AA2: 22 | | |
| 259, 260 | Unknown | Probability: 1.000 AA1: | MHKKTLLATLILGLLAGQAVA | 500 |
| | | 21 AA2: 22 | | |
| 261, 262 | Unknown | Probability: 1.000 AA1: | MHKKTLLATLILGLLAGQAVA | 500 |
| | | 21 AA2: 22 | | |
| 263, 264 | Unknown | Probability: 1.000 AA1: | MHKKTLLATLILGLLAGQAVA | 500 |
| | | 21 AA2: 22 | | |
| 265, 266 | Unknown | Probability: 1.000 AA1: | MHKKTLLATLILGLLAGQAVA | 500 |
| | | 21 AA2: 22 | | |
| 267, 268 | Unknown | Probability: 1.000 AA1: | MHKKTLLATLILGLLAGQAVA | 500 |
| | | 21 AA2: 22 | | |
| 269, 270 | Unknown | Probability: 1.000 AA1: | MHKKTLLATLVLGLLAGQAVA | 565 |
| | | 21 AA2: 22 | | |
| 271, 272 | Unknown | Probability: 1.000 AA1: | MHKKTLLATLVFGLLAGQAVA | 564 |
| | | 21 AA2: 22 | | |
| 273, 274 | Unknown | Probability: 1.000 AA1: | MHKKTLLATLILGLLAGQAVA | 500 |
| | | 21 AA2: 22 | | |
| 275, 276 | Unknown | Probability: 1.000 AA1: | MHKKTLLATLILGLLAGQAVA | 500 |
| | | 21 AA2: 22 | | |
| 277, 278 | Unknown | Probability: 1.000 AA1: | MHKKTLLATLVFGLLAGQAVA | 564 |
| | | 21 AA2: 22 | | |
| 279, 280 | Unknown | Probability: 1.000 AA1: | MHKKTLLATLVFGLLAGQAVA | 564 |
| | | 21 AA2: 22 | | |
| 281, 282 | Unknown | Probability: 1.000 AA1: | MHKKTLLATLVFGLLAGQAVA | 564 |
| | | 21 AA2: 22 | | |
| 283, 284 | Unknown | Probability: 1.000 AA1: | MHKKTLLATLVFGLLAGQAVA | 564 |
| | | 21 AA2: 22 | | |
| 285, 286 | Unknown | Probability: 1.000 AA1: | MHKKTLLATLILGLLAGQAVA | 500 |
| | | 21 AA2: 22 | | |
| 287, 288 | Unknown | Probability: 1.000 AA1: | MHKKTLLATLILGLLAGQAVA | 500 |
| | | 21 AA2: 22 | | |
| 289, 290 | Unknown | Probability: 1.000 AA1: | MHKKTLLATLILGLLAGQAVA | 500 |
| | | 21 AA2: 22 | | |
| 291, 292 | Unknown | Probability: 1.000 AA1: | MHKKTLLATLVFGLLAGQAVA | 564 |
| | | 21 AA2: 22 | | |
| 293, 294 | Unknown | Probability: 1.000 AA1: | MHKKTLLATLILGLLAGQAVA | 500 |
| | | 21 AA2: 22 | | |
| 295, 296 | Unknown | Probability: 1.000 AA1: | MHKKTLLATLIFGLLAGQAVA | 499 |
| | | 21 AA2: 22 | | |
| 299, 300 | Unknown | Probability: 1.000 AA1: | MPFTRTVLALSLGLVLLQSQVHA | 566 |
| | | 23 AA2: 24 | | |
| 301, 302 | Unknown | Probability: 1.000 AA1: | MKFTPTLLAVALAGCLSTQVQA | 567 |
| | | 22 AA2: 23 | | |
| 427, 428 | Unknown | Probability: 1.000 AA1: | MPFCRTLLALSLGLVLWQGQAHA | 554 |
| | | 23 AA2: 24 | | |
| 429, 430 | Unknown | Probability: 1.000 AA1: | MPFSRTLLAASLALLITGQAPLYA | 556 |
| | | 24 AA2: 25 | | |
| 437, 438 | Unknown | Probability: 0.826 AA1: | MKNNKCIAILGGMGPQASS | 563 |
| | | 19 AA2: 20 | | |
| 457, 458 | Unknown | Probability: 0.742 AA1: | MARVVLRWARSAYIRITTGSHALF | 568 |
| | | 30 AA2: 31 | ADATLA | |

The wild type (leadered) broad amino acid racemase (BAR) from Pseudomonas putida KT2440 BAR (cloned as described in Examples 28 in WO 2007/1033389) was not difficult to grow in the pET30 vector (Novagen, Madison, Wis.) and E. coli expression host BL21(DE3) (Novagen, Madison, Wis.). The leader sequence for the P. putida KT2440 BAR was: MPFRRTLLAASLALLITGQAPLYA (SEQ ID NO:502).

To further investigate the effect of the leader sequence on growth, some racemases were subcloned into expression vectors with and without the native signal sequences. The samples are listed below (Table 2). The left-hand column indicates the leadered sublone version while the middle column indicates the same gene subcloned without a leader sequence (for example, SEQ ID NO:412 is the leaderless version of SEQ ID NO:490).

These samples were in the pSE420-cHis vector/MB2946 host (Strych & Benedik, 2002, J. Bacteriology, 184:4321-5). The vector pSE420-cHis is a derivative of pSE420 (Invitrogen, Carlsbad, Calif.). For pSE420-cHis, the vector was cut with NcoI and HindIII, and ligated with C-His: C-His: 5′-CCA TGG GAG GAT CCA GAT CTC ATC ACC ATC ACC ATC ACT AAG CTT-3′ (SEQ ID NO:569). The expression of the His-tag in this vector depends on the choice of host and stop codon. That is, if a TAG stop codon and a supE host are used, the His-tag is expressed; if a TAG stop codon and a non-supE host are used, the His-tag is not expressed. Unless indicated otherwise, the His-tag was not expressed in these experiments.

Growth conditions were overnight in LB with 100 μg/mL carbenicillin, 37° C.

TABLE 2
Racemases that were subcloned with and without the native leader
sequence

| Subclone | Subclone | |
|---|---|---|
| w/ native | counterpart | |
| leader | w/out leader | |
| (SEQ ID NO) | (SEQ ID NO) | Comments |
| 490 | 412 | leadered version difficult to grow |
| 492 | 400 | no difficulties growing either version |
| 494 | 408 | leadered version difficult to grow |
| 496 | 410 | leadered version difficult to grow |
| 498 | 402 | leadered version difficult to grow |
| 428 | 404 | leadered version does not reach |
| | | OD600 = 0.5 within 8 hours; could not |
| | | induce |
| 430 | 406 | leadered version does not reach |
| | | OD600 = 0.5 within 8 hours; could not |
| | | induce |

In general, the leadered racemase subclones were more difficult to grow than the non-leadered counterparts under the conditions described in Part A. SEQ ID NOs:490, 494, 496 and 498 were difficult to grow. SEQ ID NOs:428 and 430 would grow; however, they grew extremely slowly and did not reach an inducible OD₆₀₀=0.5 within 8 hours. SEQ ID NO:492 was the only leadered racemase subclone tested that was not difficult to grow.

In summary, leadered racemase candidates generally were harder to grow than the non-leadered counterparts under the conditions described above. The reason for the decrease in viability or robustness has not been identified. The cells could potentially be expelling the plasmids, thereby losing the antibiotic resistance over time. In order to maximize robustness, the number of rounds of growth for racemases with leader sequences was minimized This was done by storing the DNA and performing fresh transformations each time the constructs were used.

The host organisms, expression conditions, and post expression cell handling can all affect whether there is detectable tryptophan racemase activity under the conditions of the assay in the presence of the respective leader sequences. Additionally, under optimized conditions, it is expected that racemases could have tryptophan racemase activity with or without leader sequences (native or artificial such as PelB).

### Example 2

### Improvement of SEQ ID NO:412 Solubility Using ArcticExpress™ Hosts

The expression of SEQ ID NO:411 nucleic acid encoding a racemase polypeptide having the sequence of SEQ ID NO:412 was analyzed by SDS-PAGE. SEQ ID NO:411 nucleic acid expressed well and the resulting SEQ ID NO:412 polypeptide had high activity even though only a portion (<20%) of the corresponding band in the total protein lane was soluble. In order to improve soluble expression, the racemase was moved into two ArcticExpress™ hosts (Stratagene, La Jolla, Calif.). The racemase was subcloned into the pET28b vector and the DNA was transformed into ArcticExpress™(DE3) and ArcticExpress™(DE3)RIL and plated on LB kanamycin 50 μg/mL, gentamicin 20 μg/mL, and LB kanamycin 50 μg/mL, gentamicin 20 μg/mL, streptomycin 75 μg/mL, respectively. The pET28b vector was also transformed into each host as a negative control. Samples were grown overnight at 30° C. Four colonies were picked for each construct from each ArcticExpress™ host.

TABLE 3
Names of constructs in ArcticExpress ™(DE3) &
ArcticExpress ™(DE3)RIL

| Name | Description |
|---|---|
| DE3-1 | SEQ ID NO: 411 ORF in pET28b/ArcticExpress ™(DE3)- |
| | colony #1 |
| DE3-2 | SEQ ID NO: 411 ORF in pET28b/ArcticExpress ™(DE3)- |
| | colony #2 |
| DE3-3 | SEQ ID NO: 411 ORF in pET28b/ArcticExpress ™(DE3)- |
| | colony #3 |
| DE3-4 | SEQ ID NO: 411 ORF in pET28b/ArcticExpress ™(DE3)- |
| | colony #4 |
| DE3-V | pET28b/ArcticExpress ™(DE3) |
| RIL-1 | SEQ ID NO: 411 ORF in pET28b/ArcticExpress ™(DE3)RIL- |
| | colony #1 |
| RIL-2 | SEQ ID NO: 411 ORF in pET28b/ArcticExpress ™(DE3)RIL- |
| | colony #2 |
| RIL-3 | SEQ ID NO: 411 ORF in pET28b/ArcticExpress ™(DE3)RIL- |
| | colony #3 |
| RIL-3 | SEQ ID NO: 411 ORF in pET28b/ArcticExpress ™(DE3)RIL- |
| | colony #4 |
| RIL-V | pET28b/ArcticExpress ™(DE3)RIL |

Cultures were streaked onto fresh plates with the appropriate antibiotics two days prior to performing a large scale growth. Samples were grown on LB plates with the appropriate antibiotics and incubated overnight at 30° C. The next day, a single colony was picked from each plate and used to inoculate 50 mL of LB with appropriate antibiotics. Samples were incubated overnight at 30° C. and 210 rpm. The next day, the culture was used to inoculate 500 mL of LB with the appropriate antibiotics in a 2.8 L baffled flask to an OD₆₀₀ₙₘ of 0.05. The cultures were grown at 30° C. at 210 rpm. When the OD₆₀₀ₙₘ was between 0.4-0.8, the flasks were transferred to an 11° C. incubator and allowed to incubate for 10 minutes prior to inducing with a final concentration of 1 mM IPTG Samples were induced overnight at 11° C. at 210 rpm (with the exception of DE3-2 and DE3-4, which were induced at 16° C.).

The next morning the cultures were collected and centrifuged at 6,000 rpm for 20 minutes, and the supernatant was discarded. The pellet was resuspended in 20 mL of 50 mM sodium phosphate buffer (pH 7.5), 400 μg/mL lysozyme, 26 U/mL DNaseI. Cells were lysed using a microfluidizer (Microfluidics Corporation, Newton, Mass.) per the manufacturer's instructions; each sample was passed through the microfluidizer three times. One mL of lysate was set aside for gel analysis of the total protein fraction. The remainder of the lysate was centrifuged at 12,000 rpm at 4° C. for 30 minutes. The supernatant was saved. Protein concentration was determined using the Bio-Rad Protein Assay (Bio-Rad, Hercules, Calif.). The soluble and whole cell fraction was then analyzed by SDS-PAGE using 4-20% Tris-glycine gels (Invitrogen, Carlsbad, Calif.).

TABLE 4
Approximate soluble expression levels of racemase constructs in
pET28b/ArcticExpress ™(DE3) and pET28b/ArcticExpress ™(DE3)RIL

| Name | Approximate amount of soluble expression |
|---|---|
| DE3-1 | 75% |
| DE3-2 | 75% |
| DE3-3 | 75% |
| DE3-4 | 75% |
| RIL-1 | 75% |
| RIL-2 | 50% |
| RIL-3 | 75% |
| RIL-4 | 50% |

As shown above, the soluble expression of the racemase, expressed as a percentage of the corresponding total racemase protein band, was improved in the ArcticExpress™(DE3) & ArcticExpress™(DE3)RIL host.

Samples were tested for activity using a racemase assay (as described in Example 4). Racemases were loaded at 7.5, 0.75, 0.075 μg/mL total protein and incubated with 10 mM L-tryptophan and 10 μM PLP at pH 8 and 37° C. At indicated timepoints, 50 μL of the reaction product was added to 150 μL of ice cold acetonitrile. Samples were vortexed for 30 seconds and the supernatant was then diluted fifty-fold in methanol. Samples were then analyzed by LC/MS/MS (as described in Example 4) to monitor the D-tryptophan formed and the residual L-tryptophan.

TABLE 5
Racemase activity of SEQ ID NO: 412 expressed from constructs in
ArcticExpress ™(DE3) or ArcticExpress ™(DE3)RIL

μg/ml D-tryptophan formed

| Name | 0 hrs | 1.75 hrs | 3.75 hrs | 19.75 hrs |
|---|---|---|---|---|
| DE3-1 | 13.58 | 1010 | 1488 | 1278 |
| DE3-2 | 4.24 | 1044 | 1130 | 1044 |
| DE3-3 | 4 | 1018 | 1180 | 1166 |
| DE3-4 | 5.46 | 1060 | 1070 | 1112 |
| RIL-1 | 14.2 | 1128 | 1178 | 1564 |
| RIL-2 | 13.2 | 976 | 1078 | 1110 |
| RIL-3 | 10.2 | 954 | 1122 | 1134 |
| RIL-4 | 7.98 | 1008 | 1164 | 1134 |

7.5 μg/mL total protein

As shown above, all of the constructs were active in ArcticExpress(DE3) and ArcticExpress(DE3)RIL at a 7.5 μg/mL total protein loading. All the constructs were also active when the protein was loaded at 0.75 and 0.075 μg/mL total protein. The vector/host controls had little or no activity compared to the racemase constructs.

In summary, the racemase polypeptide having the sequence of SEQ ID NO:412 was active and soluble expression was improved in ArcticExpress™(DE3) & ArcticExpress™(DE3)RIL.

### Example 3

### Activity of Racemase PFAM Domain Subclones

Several sets of proprietary degenerate PCR primers were designed as part of a sequence-based discovery effort for the amplification of racemases from mixed population environmental DNA libraries as described in U.S. Pat. No. 6,455,254. One set of proprietary degenerate PCR primers amplified the PFAM domain of the racemase exclusively. The racemases were amplified using a sequence-based discovery method as described in U.S. Pat. No. 6,455,254. The PFAM domain is slightly smaller than the full-length racemase protein. As compared to the full length racemase, the PFAM domain is missing about 30-40 amino acids from the N-terminus (mostly signal peptide) and about 10-20 amino acids from the C-terminus.

Several racemase PFAM domains were amplified using this method. Three PFAM domains were selected for subcloning in order to determine if the PFAM domain was sufficient to detect racemase activity. The samples were subcloned into the pSE420-cHis vector (His-tag not expressed) in MB2946 host cells (Strych & Benedik, 2002, J. Bacteriology, 184:4321-5). The subclones were SEQ ID NOs:425, 439 and 461 encoding SEQ ID NOs:426, 440 and 462, respectively.

The polypeptide having the sequence shown in SEQ ID NO:426 was selected for activity testing. Flasks containing 50 mL LB, 100 μg/mL carbenicillin and 50 mM D-alanine were inoculated from glycerol stocks and grown overnight at 37° C. with shaking. The following morning, flasks containing 400 mL LB, 100 μg/mL carbenicillin and 50 mM D-alanine were inoculated to an OD₆₀₀ of 0.05. Cultures were grown at 37° C. with shaking and induced with 1 mM IPTG when the OD₆₀₀ₙₘ was between 0.5-0.8. Cultures were induced overnight at 30° C.

Cell pellets were collected by centrifugation at 6000 rpm for 20 minutes. Cell pellets were resuspended in 20 mL of 50 mM sodium phosphate buffer pH 7.5 with 26 U/ml DNAseI. Cell pellets were lysed in a microfluidizer (Microfluidics Corporation, Newton, Mass.) per the manufacturer's instructions. Samples were centrifuged at 12,000 rpm for 30 minutes and the soluble fraction was collected. Protein concentration was determined by comparing the absorbance of cell extract containing the SEQ ID NO:426 polypeptide to known standards in the Bio-Rad Protein Assay reagent (Bio-Rad, Hercules, Calif.).

Samples were tested for activity using the following racemase assay conditions (also as described in Example 4). Racemases were loaded at 10 mg/mL total protein and incubated with 10 mM L-tryptophan and 10 μM PLP at pH 8 and a temperature of 37° C. At indicated timepoints (0, 2, 4, and 24 hours), 50 μL of the reaction product was added to 150 μL of ice-cold acetonitrile. Samples were vortexed for 30 seconds and passed through a 0.2 μm filter and the filtrate was then diluted fifty-fold in methanol. Samples were then analyzed by LC/MS/MS (as described in Example 4) to monitor the D-tryptophan formed (Table 6).

TABLE 6
Racemase activity of PFAM

| | | μg/ml D-tryptophan | | | |
|---|---|---|---|---|---|
| Name | Description | 0 hr | 2 hr | 4 hr | 24 hr |
| E coli MB2946 host | Negative control | 0.26 | 0.18 | 0.22 | 0.22 |
| cells | | | | | |
| Pseudomonas putida | Positive control | 0.23 | 58.97 | 88.35 | 180.41 |
| KT2440 BAR | | | | | |
| (leadered) | | | | | |
| Racemase | Racemase PFAM | 0.67 | 33.84 | 58.18 | 106.69 |
| (SEQ ID NO: 426) | | | | | |
| No enzyme | | 0.18 | 0.21 | 0.21 | 0.18 |

E. coli MB2946 host cells (Strych & Benedik, supra) was used as the negative control, while wild type Pseudomonas putida KT2440 BAR was used as a positive control. The racemase having SEQ ID NO:426 was active under the conditions described in Example 4. The results above thereby demonstrate that a racemase PFAM domain could have sufficient structural elements to maintain racemase catalytic activity.

### Example 4

### Growth and Racemase Assay Procedures Enzyme Preparation

Gycerol stocks were used to inoculate flasks containing 50 mL of LB with the appropriate antibiotic. The starter culture was grown overnight at 37° C. and 230 rpm. The OD₆₀₀ₙₘ of starter culture was checked, and used to inoculate a 400 ml culture to an OD₆₀₀ of 0.05. The culture was incubated at 37° C. and 230 rpm, and the OD₆₀₀ₙₘ was checked periodically. The cultures were induced, typically with 1 mM IPTG, when the OD₆₀₀ reached between 0.5-0.8. Induced cultures were incubated overnight at 30° C. and 230 rpm. The culture was harvested by pelleting cells at 4000 rpm for 15 minutes. The supernatant was poured off, and cell pellets were either lysed immediately or frozen for later use.

The pellets were resuspended in 20 mls of 50 mM sodium phosphate buffer (pH 7.5) supplemented with 26 U/ml of DNAse. Once the pellet was completely resuspended in the buffer, cells were lysed using a microfluidizer (Microfluidics Corporation, Newton, Mass.) per the manufacturer's instructions. The cell extract was collected and centrifuged at 11,000 rpm for 30 minutes. The supernatant was collected in a clean tube and filtered through a 0.2 μm filter. Five mL aliquots of clarified lysate were placed in each vial and freeze-dried using the lyophilizer (Virtis Company, Gardinier, N.Y.) per the manufacturer's instructions. A 1 mL sample was retained for protein estimation using the Bio-Rad Protein Assay Reagent (Bio-Rad, Hercules, Calif.) and SDS-PAGE analysis. Once the lysate was lyophilized, the amount of protein per vial was calculated.

Enzymes were prepared for the activity assay by resuspending in 50 mM sodium phosphate (pH 7.5). The racemase assays were usually run with about 10-20 mg/ml total protein.

### Racemase Assay

Ten mM L-tryptophan, 10 μM PLP, 50 mM sodium phosphate pH 8, 10 mg/mL racemase (total protein) prepared as described above (see Example 4—Enzyme Preparation) were combined and incubated at 37° C. and 300 rpm. Fifty μL of the reaction product were transferred to 150 μL of ice cold acetonitrile at timepoints (generally 0, 2, 4, and 24 hours) and the samples were vortexed for 30 seconds. The samples were centrifuged at 13,200 rpm for 10 minutes and 4° C. and the supernatant was passed through a 0.45 μm filter. The filtrate was diluted 10-fold in methanol. Samples were analyzed by LC/MS/MS to monitor the D-tryptophan formed (see description below).

### LC/MS/MS Method for Detecting D- and L-Tryptophan

LC/MS/MS screening was achieved by injecting samples from 96-well plates using a CTCPal auto-sampler (LEAP Technologies, Carrboro, N.C.) into a 70/30 MeOH/H₂O (0.25% AcOH) mixture provided by LC-10ADvp pumps (Shimadzu, Kyoto, Japan) at 0.8 mL/min through a Chirobiotic T column (4.6×250 mm) and into the API4000 TurboIon-Spray triple-quad mass spectrometer (Applied Biosystems, Foster City, Calif.). Ion spray and Multiple Reaction Monitoring (MRM) were performed for the analytes of interest in the positive ion mode and each analysis lasted 15.0 minutes. D- and L-tryptophan parent/daughter ions: 205.16/188.20.

### Example 5

### Racemase Activity Dependent Upon Conditions

SEQ ID NOs:401, 385, 395, 413, 419, 421, 425, 437, 427, 433, and 435 are racemase subclones that, when expressed (into polypeptides having the sequence of SEQ ID NOs:402, 386, 396, 414, 420, 422, 426, 438, 428, 434 and 436, respectively) were active under the conditions described in Part A. These subclones were not active under the conditions described in Part B (see Example 6 for details on SEQ ID NOs:385, 395, and 401; see Example 7 for details on SEQ ID NO:413; see Example 12 for details on SEQ ID NOs:419, 421, 425, 427, 433, 435, and 437).

The racemase subclones were in the pSE420-cHis vector/MB2946 host (Strych & Benedik, 2002, J. Bacteriology, 184:4321-5) with the exception of SEQ ID NO:413. SEQ ID NO:413 was in pSE420-cHis/Top10 host (Invitrogen, Carlsbad, Calif.). The His-tag was not expressed in any of these subclones.

The subclones were grown, lysed and lyophilized according to the procedures described in Example 4. Samples were tested for activity using a racemase assay (as described in Example 4). Racemases were incubated with 10 mM L-tryptophan and 10 μM PLP at pH 8 and 37° C. All racemases were utilized at 10 mg/mL total protein concentration with the exception of the polypeptide having the sequence of SEQ ID NO:402. This polypeptide having the sequence of SEQ ID NO:402 was used at 5 mg/mL total protein concentration because there was not enough biomass to allow for a higher loading.

At indicated timepoints, 50 μL of the reaction product was added to 150 μL of ice cold acetonitrile. Samples were vortexed for 30 seconds and the supernatant was then diluted fifty-fold in methanol. Samples were then analyzed by LC/MS/MS (as described in Example 4) to monitor the D-tryptophan formed and the residual L-tryptophan remaining.

Tables 7, 8, 9 and 10 show the racemase activity over time. Samples that were assayed together are grouped together in a single table.

TABLE 7
Racemase activity assays

| | μg/ml D-tryptophan formed | | | | | | | | |
|---|---|---|---|---|---|---|---|---|---|
| Time | SEQ ID | SEQ ID | SEQ ID | SEQ ID | SEQ ID | SEQ ID | No | (−) | (+) |
| (hours) | NO: 414 | NO: 420 | NO: 422 | NO: 434 | NO: 436 | NO: 438 | enzyme | control | control |
| 0 | 0.19 | 0.96 | 0.96 | 0.20 | 0.18 | 22.35 | 0.18 | 0.26 | 0.23 |
| 2 | 33.23 | 309.78 | 187.81 | 1.63 | 0.21 | 38.70 | 0.21 | 0.18 | 58.97 |
| 4 | 51.87 | 320.22 | 191.46 | 2.92 | 2.85 | 71.35 | 0.21 | 0.22 | 88.35 |
| 24 | 119.45 | 344.74 | 128.80 | 8.01 | 4.19 | 101.03 | 0.18 | 0.22 | 180.42 |

TABLE 8
Racemase activity assay

| Time | μg/ml D-tryptophan formed | | |
|---|---|---|---|
| (hours) | SEQ ID NO: 402 | (−) control | (+) control |
| 0 | 0.00 | 1.85 | 8.29 |
| 2 | 19.67 | 0.00 | 583.79 |
| 4 | 18.25 | 0.00 | 715.26 |
| 24 | 44.05 | 4.32 | 730.14 |

TABLE 9
Racemase activity assay

| | μg/ml D-tryptophan formed | | |
|---|---|---|---|
| Time (hours) | SEQ ID NO: 396 | (−) control | (+) control |
| 0 | 3.51 | 0.00 | 0.00 |
| 2 | 10.58 | 0.00 | 189.04 |
| 4 | 12.29 | 0.00 | 231.84 |
| 24 | 63.49 | 0.63 | 609.44 |

TABLE 10
Racemase activity assay

| | μg/ml D-tryptophan formed | | |
|---|---|---|---|
| Time (hours) | SEQ ID NO: 386 | (−) control | (+) control |
| 0 | 0.00 | 0.00 | 0.80 |
| 2 | 0.00 | 0.00 | 189.04 |
| 4 | 6.03 | 0.00 | 230.98 |
| 24 | 5.23 | 3.10 | 478.75 |

In summary, racemases having the sequence shown in SEQ ID NOs:402, 386, 396, 414, 420, 422, 426, 438, 434, and 436 were active on tryptophan under the conditions described in Part A. These samples were not active under the conditions described in Part B (see Examples 6, 7, and 12). The differences in observed racemase activity may be attributed to differences in host strains, expression conditions, post-expression cell handling and assay protein-loading. Refer to Example 3 for activity data for a racemase having SEQ ID NO:426. It is noted that the racemase having SEQ ID NO:428 is not included here because it did not reach an inducible OD₆₀₀ and, therefore, was not induced.

It is expected that the presence of activity in a polypeptide encoded from a subcloned nucleic acid is predictive of the presence of activity in the corresponding polypeptide encoded from the full-length or wild type nucleic acid. See, for example, Table 11.

TABLE 11

| Subclone number (SEQ ID NO) | Wild type clone (SEQ ID NO) |
|---|---|
| 401 | 51 |
| 385 | 35 |
| 395 | 9 |
| 413 | 7 |
| 419 | 115 |
| 421 | 117 |
| 425 | 121 |
| 437 | 103 |
| 433 | 55 |
| 435 | 113 |

### Part B

### Example 6

### Analysis of Racemases Provided as pSE420 Clones

SEQ ID NOs:385, 387, 389, 391, 393, 395, 397, 399, 401, 403, 405, 407, and 409 encoding racemases having SEQ ID NOs:386, 388, 390, 392, 394, 396, 398, 400, 402, 404, 406, 408 and 410, respectively, were provided as pSE420-cHis clones. One skilled in the art can synthesize the genes encoding these racemases using various published techniques for example, as described in Stemmer et al., 1995, Gene, 164(1):49-53. The plasmids were transformed into E. coli XL-1 Blue (Novagen/EMD Biosciences, San Diego, Calif.) cells as per manufacturer instructions.

Transformants were grown overnight at 37° C. and 250 rpm in 5 ml LB containing ampicillin (100 μg/mL). Overnight cultures were used to inoculate 25 mL of the same media in 250 mL baffled shake flasks. Cultures were grown at 30° C. and 250 rpm until they reached an OD₆₀₀ of 0.6, after which protein expression was induced with 1 mM IPTG for 4.25 h at 30° C. Samples for total protein were taken prior to induction and right before harvesting. Cells were harvested by centrifugation and frozen at −80° C.

Cell extracts were typically prepared from the above frozen pellets by adding 5 ml per g of cell pellet of Bugbuster Amine Free (Novagen/EMD Biosciences, San Diego, Calif.) with 5 μL/mL of Protease Inhibitor Cocktail II (Calbiochem, San Diego, Calif.) and 1 μl/ml of benzonase nuclease (Novagen/EMD Biosciences, San Diego, Calif.). Cell solutions were incubated at room temperature with gentle mixing for 15 min; cells were spun out at 14,000 rpm for 20 min (at 4° C.) and the supernatant was carefully removed. Detergents and low molecular weight molecules were removed by passage through PD-10 columns (GE Healthcare, Piscataway, N.J.) previously equilibrated with 100 mM potassium phosphate (pH 7.8) with 0.05 mM PLP. Proteins were eluted with 3.5 mL of the same buffer. Total protein concentration was determined using the Pierce BCA total protein assay with bovine serum albumin (BSA) as the standard, per the manufacturer's instructions (Pierce Biotechnology, Inc., Rockford, Ill.). The resulting cell-free extract was used for subsequent assays.

Racemase assays were performed on crude protein extracts as described in Example 17. For the tryptophan racemase assay, desalted protein was added to target 50 μg racemase protein for each enzyme assay. Calculations were based on Pierce BCA total protein analysis with BSA as the standard (Pierce Biotechnology, Inc., Rockford, Ill.) and SDS-PAGE visual inspection. Formation of D-tryptophan was measured at 2 hours, and 21 hours. A cell-free extract of empty vector pSE420-cHis served as a negative control.

TABLE 12
D-tryptophan production

| Racemase | μg/mL D-trp | μg/mL D-trp |
|---|---|---|
| polypeptide (SEQ ID NO) | 2 hours | 21 hours |
| 17148 - pSE420-cHis | nd | nd |
| vector control | | |
| 386 | nd | nd |
| 388 | nd | nd |
| 390 | nd | nd |
| 392 | nd | nd |
| 394 | nd | 6 |
| 396 | nd | nd |
| 398 | nd | nd |
| 414 | nd | nd |
| 400 | 46 | 410 |
| 402 | nd | nd |
| 404 | 10 | 18 |
| 406 | 1171 | 2724 |
| 408 | 248 | 785 |
| 410 | 502 | 1435 |
| A. caviae wild-type BAR | 1695 | 3820 |

nd = not detected under the conditions of the assay as described above

It is noted that, when cell-free extracts were analyzed by SDS-PAGE, very low expression was observed. It was concluded, therefore, that the cell-free extracts likely contained significantly less protein than the purified positive control enzyme (wild-type A. caviae), prepared as described in Example 30 of WO 07/103,389 and as described in Example 19.

Tryptophan racemase activity was detected for racemases having the amino acid sequence shown in SEQ ID NOs:400, 404, 406, 408 and 410 using the conditions described in Part B. Similar results were obtained for racemases having the polypeptide sequence shown in SEQ ID NO:400, 404, 406, 408 and 410 using the reaction conditions described in Part A. In addition, detectable activity was observed for candidates having the polypeptide sequence shown in SEQ ID NO:386, 396 and 402 using conditions described in Part A, but was not observed using conditions described in Part B (see, for example, Example 5). Detectable activity was not observed for the polypeptide shown in SEQ ID NO:394 under the conditions described in Part A, while very low activity (barely detectable at 21 hours) was observed for the racemase polypeptide shown in SEQ ID NO:394 under the conditions described in Part B.

Some constructs were observed, under the conditions described in Part A, to be unstable in expression systems, particularly those with a leader sequence. The host organisms, expression conditions, and post expression cell handling can all affect whether there is detectable tryptophan racemase activity under the conditions of the assay. Additionally, under optimized conditions, it is expected that all racemase candidates could have tryptophan racemase activity.

It is expected that the presence of activity in a polypeptide encoded from a subcloned nucleic acid is predictive of the presence of activity in the corresponding polypeptide encoded from the full-length or wild type nucleic acid, as indicated below in Table 13.

TABLE 13

| Subclone number (SEQ ID NO) | Wild-type clone (SEQ ID NO) |
|---|---|
| 399 | 41 |
| 403 | 53 |
| 405 | 57 |
| 407 | 47 |
| 409 | 45 |
| 393 | 3 |

### Example 7

### Characterization of Racemases Having the Sequences Shown in SEQ ID NO:414 and 412

Racemases having the polypeptide sequence of SEQ ID NO:412 and 414 were both found to be active when assayed for tryptophan racemase activity under the conditions described in Part A. One skilled in the art can synthesize the genes encoding these racemases using various published techniques for example, as described in Stemmer et al., supra. It should be noted that 10 mg of total protein in the form of lyophilized cell extracts was used in Part A when evaluating racemase activity (see Example 4). In some cases, this was ten times as much total soluble protein as was used in the assays described in Part B. This difference in the amount of protein used in the assays (i.e., of Part A vs. Part B) may explain, at least in part, some of the differences in activity observed with the same polypeptide.

The nucleic acid having the sequence of SEQ ID NO:413, which encodes the racemase polypeptide having the sequence shown in SEQ ID NO:414, was expressed in 3 different hosts in Part A (MB2946, XL-1 Blue, and TOP10). High activity was observed in cell-free extract from the TOP10 host, with only a small amount of activity observed in XL-1 Blue and no detectable product formed from the MB2946 host under the conditions of the assay. The nucleic acid having the sequence of SEQ ID NO:411, which encodes the polypeptide having the sequence of SEQ ID NO:412, was expressed in the MB2946 host and found to be highly active.

The nucleic acids of SEQ ID NOs:411 and 413 were received as pSE420-cHis constructs, and were initially evaluated in E. coli TOP10. Strains were grown and induced, and cell extracts were prepared as described in Part B.

Tryptophan racemase assays were carried out using desalted cell-free extracts under the conditions described in Example 17.

100 μg purified A. caviae D76N, prepared as described in Example 19, served as a positive control for the assay, and cell-free extract of E. coli host cells containing the empty vector pSE420-cHis served as a negative control. 1.4 mg of total protein was used for polypeptides having SEQ ID NO:412 and 414.

TABLE 14
Trp Assay results

| | D-trp production, μg/mL | | | |
|---|---|---|---|---|
| | pSE420-cHis (vector | A. caviae | SEQ ID | SEQ ID |
| Time (Hr) | control) | D76N | NO: 414 | NO: 412 |
| 0 | nd | nd | nd | nd |
| 0.5 | 9 | 1797 | nd | 1205 |
| 1 | 12 | 3764 | nd | 1765 |
| 4 | 24 | 3818 | 2 | 3012 |
| 22 | 22 | 3621 | 33 | 2204 |

nd = not detected under the conditions of the assay as described above;
control was purified BAR from A. caviae D76N mutant - 100 μg/ml;
not much activity detected in crude extract of SEQ ID NO: 414 polypeptides (1.4 mg/ml);
some limited (very low) activity in extracts containing pSE420-cHis vector control;
no band was observed in crude extracts from SEQ ID NO: 412 polypeptides, however, good racemase activity was still detected.

There was very little activity detected in crude extract that contains the polypeptide having the sequence shown in SEQ ID NO:414 as well as negative control. The polypeptide having the sequence shown in SEQ ID NO:412 gave high specific activity given that there was barely detectable protein band observed in the soluble fraction (comparing 100 μg of purified A. caviae BAR to an estimated less than 30 μg of SEQ ID NO:412 polypeptide, assuming it was 2% or less of the total protein).

The host organisms, expression conditions, and post expression cell handling can all affect whether there is detectable tryptophan racemase activity under the conditions of the assay. Additionally, under optimized conditions, it is expected that all racemase candidates could have tryptophan racemase activity.

It is expected that the presence of activity in a polypeptide encoded from a subcloned nucleic acid is predictive of the presence of activity in the corresponding polypeptide encoded from the full-length or wild type nucleic acid, as indicated in Table 15 below.

TABLE 15

| Subclone number (SEQ ID NO) | Wild type clone (SEQ ID NO) |
|---|---|
| 413 | 7 |
| 411 | 61 |

### Example 8

### Polypeptide Having the Sequence of SEQ ID NO:412 is More Active on Tryptophan Than Alanine

In order to get a more quantitative comparison of SEQ ID NO:412 to the benchmark BAR (A. caviae D76N from Example 19), SEQ ID NO:411 (encoding SEQ ID NO:412) was PCR-amplified with NcoI and XhoI restriction sites for subcloning into pET28 (Novagen/EMD Chemicals, San Diego, Calif.).

TABLE 16

| | | SEQ |
|---|---|---|
| | | ID |
| Designation | Sequence (5′ → 3′) | NO: |
| SEQ ID | GGTTCCGGAACCATGGCCGAAACAAATCTGC | 503 |
| NO: 411-F | | |
| Nco1 | | |
| SEQ ID | GGTTCCAAGGCTCGAGCTATTTTTGTTCTGCTATTCTATATGTC | 504 |
| NO: 411-R | | |
| Xho1 with | | |
| stop | | |
| SEQ ID | GGTTCCAAGGCTCGAGTTTTTGTTCTGCTATTCTATATGTC | 505 |
| NO: 411-R | | |
| Xho1 | | |

The pET28 constructs were created with and without a C-terminal His tag (tagged constructs were created by using a reverse primer without a stop codon in the PCR). In addition, pET26b constructs were created with a C-terminal His tag. Constructs were sequenced for accuracy (Agencourt Bioscience Inc., Beverly Mass.) and used to transform BL21(DE3) (Novagen/EMD Biosciences, San Diego, Calif.).

Transformants were grown and induced in OvernightExpress™ media and cell-free extracts were prepared as described herein. Proteins were purified from tagged constructs on Novagen/EMD Biosciences His-bind columns (Novagen/EMD Biosciences, San Diego, Calif.) and desalted on PD-10 columns; for untagged constructs, cell-free extracts were desalted on PD-10 columns.

Protein concentrations were determined by Pierce BCA protein assay and racemase purity was determined by Experion Automated Gel System (Experion, version A.01.10, Biorad, Hercules, Calif.). Racemase assays were performed on purified and crude protein extracts as described in Example 17. Racemase expression in the pET26b construct was lower than the pET28 vector, however, active protein having the sequence shown in SEQ ID NO:412 was obtained. Results for SEQ ID NO:411/pET28 (encoding the polypeptide having the sequence of SEQ ID NO:412) are shown in this example.

TABLE 17
D-trp production (μg/mL)

| | A. caviae | SEQ ID NO: 411/ | SEQ ID NO: |
|---|---|---|---|
| Time | D76N | pET28 CFE | 411/pET28 |
| (Hr) | (100 μg) | (100 μg) | purified (30 μg) |
| 0 | nd | nd | 70 |
| 0.5 | 1114 | 465 | 3133 |
| 1 | 3487 | 791 | 3350 |
| 2 | 4029 | 1113 | 4485 |

*Note 30 μg of the purified protein having the sequence of SEQ ID NO: 412 (encoded by SEQ ID NO: 411/pET28) was used as compared to 100 μg of other enzyme preps;
nd = not detected under the conditions of the assay as described above;
not normalized for racemase loading

Purified protein having the sequence shown in SEQ ID NO:412 from construct in pET28 was further characterized for racemase activity on tryptophan, alanine, and monatin. Tryptophan, monatin, and alanine assays were performed as described in Example 17, with A. caviae D76N serving as positive control for racemization assays. The analytical methodology is described in Example 18. It should be noted that, at the time these analyses were performed, the analysis of D-alanine was less quantitative than the analysis for D-tryptophan.

Racemase activity of SEQ ID NO:412 for tryptophan and alanine

TABLE 18
D-Trp nmoles/μl/μg protein

| Time (min) | A. caviae D76N | SEQ ID NO: 412 purified |
|---|---|---|
| 0 | nd | nd |
| 5 | 59 | 353 |
| 10 | 123 | 500 |
| 20 | 128 | 715 |
| 60 | 305 | 2145 |

TABLE 19
D-Ala nmoles/μl/μg protein

| Time (min) | A. caviae D76N | SEQ ID NO: 412 purified |
|---|---|---|
| 0 | 44 | nd |
| 5 | 1320 | nd |
| 10 | 2239 | 23 |
| 20 | 2602 | 314 |
| 60 | 4654 | 1044 |

nd = not detected under the conditions of the assay as described above

SEQ ID NO:412 consistently gave higher D-trp activity than the control racemase candidate, BAR, A. caviae D76N. SEQ ID NO:412 appears to have a higher preference for tryptophan versus alanine as a substrate for racemization. In contrast, A. caviae D76N BAR while active on tryptophan, has a preference for alanine as a substrate. The ability of purified SEQ ID NO:412 to racemize 7 additional L-amino acids was evaluated and the details are reported in Example 10.

In addition, the impact of alanine on tryptophan racemase activity was investigated. An experiment was designed to determine the impact of L-alanine on the racemization of L-tryptophan by either BAR A. caviae D76N or racemase polypeptide having the sequence of SEQ ID NO:412. Racemase enzymes were assayed in the presence of tryptophan and alanine together to further characterize substrate preference/competition. Assay was carried out as described in Example 17, with 30 mM of each substrate (L-Trp and L-Ala) in the reaction. For both racemase enzymes, control racemase assays were conducted in the presence of L-tryptophan alone. The data from these control assays at various time points were considered to be 100% when compared with the respective data from assays with both amino acids.

### Competition of L-Ala and L-Trp in Racemase Assay

TABLE 20
% D-trp formed (100% without L-Ala assumed)

| | | A. caviae | SEQ ID | SEQ ID NO: |
|---|---|---|---|---|
| | A. caviae | D76N (Trp | NO: 412 | 412 purified |
| Time (min) | D76N | only control) | purified | (Trp only control) |
| 0 min | | | 65% | 100% |
| 5 min | 0% | 100% | 39% | 100% |
| 20 min | 54.6% | 100% | 96% | 100% |
| 60 min | 36.7% | 100% | 94% | 100% |
| 180 min | 36.4% | 100% | 110.6% | 100% |

Despite some initial inhibition of tryptophan racemization between zero and five minutes, the polypeptide having the sequence of SEQ ID NO:412 had little to no impact on L-alanine. The SEQ ID NO:414 polypeptide retained 96%-100% of its tryptophan racemase activity between 20 minutes to the end of the assay at three hours. In contrast, BAR A. caviae D76N only retained 37%-55% of its tryptophan racemase activity in the presence of L-alanine, during the same time period. Thus, the preference of the racemase having SEQ ID NO:414 for tryptophan as a substrate is advantageous in the presence of competing substrates like alanine.

### Example 9

### Racemases Lacking Monatin Racemization Activity

TABLE 21
SEQ ID NO: 412 and A. caviae D76N do not racemize monatin

| | | | Time | Monatin Isomer Ratio | | |
|---|---|---|---|---|---|---|
| BAR | BAR ug | Vol Ptn | (hr) | % S,S | % R,S | % R,R |
| A. caviae (D76N) | 50 | 11.9 μL | 0 | 1.0 | 0.9 | 98.1 |
| SEQ ID NO: 412 | 50 | 73 μL | 0 | 0.6 | 0.8 | 98.6 |
| SEQ ID NO: 412 | 50 | 98 μL | 0 | 1.0 | 1.1 | 97.9 |
| Negative control | 0 | 0 | 0 | 0.4 | 0.1 | 98.5 |
| A. caviae (D76N) | 50 | | 24 | 0.6 | 1.0 | 98.4 |
| SEQ ID NO: 412 | 50 | | 24 | 0.5 | 1.0 | 98.5 |
| SEQ ID NO: 412 | 50 | | 24 | 0.4 | 1.3 | 98.3 |
| Negative control | 0 | | 24 | 0.6 | 0.9 | 98.5 |
| A. caviae (D76N) | 50 | | 48 | 0.4 | 0.7 | 98.9 |
| SEQ ID NO: 412 | 50 | | 48 | 0.4 | 0.8 | 98.8 |
| SEQ ID NO: 412 | 50 | | 48 | 1.3 | 0.5 | 98.2 |
| Negative control | 0 | | 48 | 0.4 | 0.7 | 98.9 |

Neither SEQ ID NO:412 nor the benchmark A. caviae BAR showed detectable racemization of R,R monatin under the conditions of the assay as described in Example 17.

TABLE 22
Racemase Substrate Specificity

| | | R,R | |
|---|---|---|---|
| Designation | Alanine | monatin | Vector/Host |
| Reference, A. caviae | +++ | − | pET30/BL21DE3 |
| SEQ ID NO: 412 | − (low, prefers | − | pSE420-cHis/TOP10, |
| | trp) | | pET28/BL21DE3 |
| SEQ ID NO: 442 | + | − | pSE420-cHis/TOP10 |
| SEQ ID NO: 456 | + | − | pSE420-cHis/TOP10 |
| SEQ ID NO: 458 | + | − | pSE420-cHis/TOP10 |
| SEQ ID NO: 462 | + | − | pSE420-cHis/TOP10 |
| SEQ ID NO: 464 | + | − | pSE420-cHis/TOP10 |
| SEQ ID NO: 466 | + | − | pSE420-cHis/TOP10 |
| SEQ ID NO: 468 | + | − | pSE420-cHis/TOP10 |
| SEQ ID NO: 470 | + | − | pSE420-cHis/TOP10 |
| SEQ ID NO: 472 | +++ | − | pSE420-cHis/TOP10 |
| SEQ ID NO: 478 | + | − | pSE420-cHis/TOP10 |
| SEQ ID NO: 314 | + | − | pET30/BL21DE3 |
| SEQ ID NO: 326 | + | − | pET30/BL21DE3 |
| SEQ ID NO: 340 | not tested | − | pET30/BL21DE3 |
| SEQ ID NO: 342 | + | − | pET30/BL21DE3 |
| SEQ ID NO: 344 | + | − | pET30/BL21DE3 |
| SEQ ID NO: 318 | + | − | pET30/BL21DE3 |
| SEQ ID NO: 330 | + | − | pET30/BL21DE3 |
| SEQ ID NO: 322 | + | − | pET30/BL21DE3 |
| SEQ ID NO: 324 | + | − | pET30/BL21DE3 |
| SEQ ID NO: 328 | + | − | pET30/BL21DE3 |
| SEQ ID NO: 346 | + | − | pET30/BL21DE3 |
| SEQ ID NO: 348 | + | − | pET30/BL21DE3 |
| SEQ ID NO: 334 | + | − | pET30/BL21DE3 |
| SEQ ID NO: 350 | not tested | − | pET30/BL21DE3 |
| SEQ ID NO: 352 | + | − | pET30/BL21DE3 |

− indicates no detectable racemization under the conditions of the assays after a minimum of 24 hours;
*indicates enzymes that were re-cloned in pET30a with a C-terminal His tag for purification and more quantitative assays

### Example 10

### The Polypeptide Having the Sequence of SEQ ID NO:412 is a Broad Specificity Amino Acid Racemase

The ability of purified SEQ ID NO:412 polypeptide to racemize 7 additional L-amino acids was evaluated. The amino acid racemase assay was carried out as described in Example 17, with 30 mM of each L-amino acid substrate and approximately 1 μg of purified racemase polypeptide SEQ ID NO:412 (expressed from SEQ ID NO:411/pET28/BL21(DE3) induction) added for each amino acid substrate assayed.

TABLE 23
Additional substrates for SEQ ID NO: 412

| | μg/mL corresponding | |
|---|---|---|
| Starting substrate (L- | D-amino acid | Relative Activity (Trp |
| amino acid) | produced (2 hours) | taken as 100%) |
| Leucine | 2429.2 | 131.8% |
| Phenylalanine | 2193.5 | 119% |
| Tryptophan | 1843.5 | 100% |
| Methionine | 1387.7 | 75.3% |
| Tyrosine | 154.1 | 8.36% |
| Alanine | 132.3 | 7.18% |
| Lysine | 49.3 | 2.67% |
| Aspartic acid | 23 | 1.25% |
| Glutamate | 1.9 | 0.1% |

The polypeptide having the sequence of SEQ ID NO:412 appears to be an amino acid racemase with broad substrate specificity and seems to prefer bulky, hydrophobic amino acids.

Racemase activity for various amino acids as substrates was observed as follows, under the conditions of the assay as described: [Leucine/Phenylalanine/Tryptophan/Methionine]>[Tyrosine/Alanine]>[Lysine/Aspartic Acid]>Glutamate.

It should be noted that analytical methods for detection of all of the above D-amino acids with the exception of tryptophan are semi-quantitative so these results indicate a trend in racemase activity.

### Example 11

### Methods to Improve Solubility of an Insoluble Protein and its Activity on Tryptophan

The polypeptide having the sequence of SEQ ID NO:412 showed lower solubility than other racemase candidates described in this application, under the expression conditions tested. The insoluble fraction of the SEQ ID NO:412 polypeptide was tested for racemization activity on tryptophan.

Cell-free extracts of SEQ ID NO:411/pET28 were prepared from frozen cell pellets by adding 5 ml of Bugbuster Amine Free (Novagen/EMD Biosciences, San Diego, Calif.) with 5 μL/mL of Protease Inhibitor Cocktail II (Calbiochem, San Diego, Calif.) and 1 μl/ml of benzonase nuclease (Novagen/EMD Biosciences, San Diego, Calif.), per gm of cell pellet. Cell pellet suspensions were incubated at room temperature with gentle mixing for 15 min; cells pellets were spun out at 14000 rpm for 20 min (at 4° C.) and retained for assays.

Cell pellets containing insoluble SEQ ID NO:412 polypeptide were washed multiple times in phosphate buffered saline to remove traces of supernatant containing soluble SEQ ID NO:412 protein fraction. Washed pellets were used in qualitative tryptophan assays (amount of protein in assay was not quantitated; rather, a set volume of pellet resuspended in phosphate buffer was added to assay). The experiment was performed twice, once with pellets that were washed four times, and the second time with frozen pellets that were thawed and washed an additional six times. Tryptophan racemization assays were performed on the insoluble protein suspension as described in Example 17.

TABLE 24
Insoluble protein assays for polypeptides having the sequence of
SEQ ID NO: 412 - active pellets
A. D-trp production (μg/mL) in pellets washed 10X and after freeze thaw

| Time (hr) | pET28 - 2 uL | pET28 - 20 uL |
|---|---|---|
| 0 | nd | nd |
| 0.16 | 44 | 353 |
| 1 | 137 | 2437 |

B. D-trp production (μg/mL) in pellets washed 4X with Phosphate buffer

| Time (hr) | pET28 2 uL | pET28 20 uL |
|---|---|---|
| 0 | nd | 19 |
| 0.16 | 153 | 681 |
| 1 | 831 | 2225 |

nd = not detected under the conditions of the assay as described above

SDS-PAGE analysis of cell pellets/insoluble protein fraction from the Bugbuster protocol above, showed a predominant protein band at the expected size (56.3 kD) for the racemase having the sequence of SEQ ID NO:412. The insoluble SEQ ID NO:412 protein fraction was observed to have tryptophan racemase activity. D-tryptophan production in the case of 20 μl samples was comparable between the two trials. The variation observed in the case of the 2 μl samples could be attributed to the small volume and sample nature (insoluble protein suspension).

Preliminary investigations indicated that the polypeptide having the sequence of SEQ ID NO:412 is not a membrane associated protein, which might be a possibility given the lack of solubility but the presence of activity.

### Experiments to Improve Solubility of the SEQ ID NO:412 Polypeptide

Various host systems reported to improve soluble expression of heterologous proteins were investigated in an effort to improve soluble expression of the SEQ ID NO:412 polypeptide: E. coli KRX (Promega, Madison, Wis.), CopyCutter™ EPI400™ (Epicentre Biotechnologies, Madison, Wis.), ArcticExpress™ (Stratagene, La Jolla, Calif.), E. coli HMS174 (Novagen/EMD Biosciences, San Diego, Calif.), and E. coli EE2D.

### A. Induction in ArcticExpress™

Competent cells of ArcticExpress™(DE3) were transformed with SEQ ID NO:411/pET28 and SEQ ID NO:411/pET26b as per manufacturer's protocol (Stratagene, La Jolla, Calif.).

Transformants were grown in LB containing kanamycin (50 mg/L) and gentamycin (20 mg/L) overnight at 37° C. and 250 rpm. A 2% inoculum was transferred to 50 mL of Novagen OvernightExpress™ AutoinductionSystem 2 (EMD Biosciences/Novagen catalog #71366) containing solutions 1-6, which contains kanamycin and gentamycin. Flasks were grown for 1.5 days at 15° C. and 250 rpm. Cells were harvested and cell extracts prepared as described herein. SDS-PAGE analysis of total and soluble protein was conducted.

No improvement was seen in solubility in the ArcticExpress™ strain. However, the chaperonin proteins that should be overexpressed in this strain were not observed (expected sizes of 10 kDa and 60 kDa) on the SDS-PAGE gel. The experiment was repeated with fresh competent cells and induction over 3 days, but SDS results were identical.

When the ArcticExpress™ experiments were repeated with the SEQ ID NO:411/pET28 construct using the methods of Part A, the data showed an improvement in soluble protein expression (see Example 1).

B. Induction in E. coli CopyCutter™

CopyCutter™ EPI400™ cells were transformed with SEQ ID NO:411/pET28 as per manufacturer instructions (Epicentre Biotechnologies, Madison, Wis.). Liquid cultures of transformants were grown overnight (LB kanamycin 50, 37° C., 250 rpm) and used to inoculate shake flasks containing 25 mL LB media, kanamycin (50 mg/L) and 1× CopyCutter™ induction solution. Cultures were grown at 30° C. and 250 rpm for 5 hours. Cultures were harvested and cell extracts were prepared as described herein. SDS-PAGE analysis of total and soluble protein was conducted.

### C. Induction in HMS174 and EE2D DE3

E. coli HMS174 (Novagen/EMD Biosciences, San Diego, Calif.) and E. coli BW30384(DE3) ΔompTΔmetE (“E. coli EE2D”) competent cells were transformed with SEQ ID NO:411/pET28 nucleic acid. (Construction of the E. coli BW30384(DE3) ΔompTΔmetE expression host and the transformation protocol are described in WO 2006/066072. Liquid cultures of transformants were grown overnight (LB kanamycin 50, 37° C., 250 rpm) and used to inoculate 50 mL flasks of Novagen OvernightExpress AutoinductionSystem 2 (EMD Biosciences/Novagen catalog #71366) containing solutions 1-6 and 50 mg/L kanamycin (25 mL in each flask). Cultures were grown at 30° C. and 250 to an OD₆₀₀ₙₘ>10. Cultures were harvested and cell extracts were prepared as described herein. SDS-PAGE analysis of total and soluble protein was conducted.

In all cases described above, no significant increase in soluble expression of the SEQ ID NO:412 polypeptide was observed based on SDS-PAGE analyses. In addition, the nucleic acid encoding the SEQ ID NO:412 polypeptide (i.e., SEQ ID NO:411) was subcloned into a derivative of the pET23d vector (Novagen, Madison, Wis.) containing the E. coli metE gene and promoter inserted at the NgoMIV restriction site and a second PsiI restriction site that was added for facile removal of the beta-lactamase gene (bla). The construction of this vector is described in WO 2006/066072. This construct was transformed into E. coli B834 DE3 host system (Novagen/EMD Biosciences, San Diego, Calif.), without significant increase in soluble expression.

Since the nucleic acid encoding SEQ ID NO:412, with its native leader sequence, could not be successfully cloned and propagated under the conditions described in Part A, a N-terminal alanine residue was added in place of the native leader sequence of SEQ ID NO:412. It was determined that deletion of this additional alanine residue had no impact on soluble expression, based on SDS-PAGE analysis.

The presence of DTT was shown to minimize protein precipitation during purification of selected histidine-tagged D-aminotransferase candidates. The addition of 5 mM DTT during the bugbuster solubilization and subsequent purification of histidine-tagged SEQ ID NO:412 from induction of SEQ ID NO:411/pET28 in BL21DE3 did not impact soluble expression as observed on SDS-PAGE.

One skilled in the art could employ various methods reported in the literature to improve soluble expression of the protein.

### Example 12

### Analysis of Racemases Provided as pSE420-cHis Clones

The nucleic acids encoding SEQ ID NO:412, 416, 418, 420, 422, 424, 426, 428, 430, 432, 434, 436, 438 and 440 racemases were provided as pSE420-cHis clones. One skilled in the art could synthesize the genes encoding these racemases using various published techniques for example, as described in Stemmer et al., supra. The plasmids were transformed into TOP10 chemically competent cells (Invitrogen, Carlsbad, Calif.). Overnight cultures grown in LB carbenicillin (100 μg/ml) were diluted a hundred-fold in 50 ml LB carbenicillin (100 μg/ml) in a 250 ml baffled flask. Cultures were grown at 30° C. with agitation at 250 rpm until they reached an OD₆₀₀ of 0.5 to 0.8, after which protein expression was induced with 1 mM IPTG for 4 h at 30° C. Samples for total protein were taken prior to induction and right before harvesting. Cells were harvested by centrifugation. Cells were frozen at −80° C.

Cell extracts were typically prepared from the above frozen pellets by adding 5 ml per g of cell pellet of Bugbuster Amine Free (Novagen/EMD Biosciences, San Diego, Calif.) with 5 μL/mL of Protease Inhibitor Cocktail II (Calbiochem, San Diego, Calif.) and 1 μl/ml of benzonase nuclease (Novagen/EMD Biosciences, San Diego, Calif.). Cell solutions were incubated at room temperature with gentle mixing for 15 min; cells were spun out at 14000 rpm for 20 min (at 4° C.) and the supernatant was carefully removed. Detergents and low molecular weight molecules were removed by passage through PD-10 columns (GE Healthcare, Piscataway, N.J.) previously equilibrated with 100 mM potassium phosphate (pH 7.8) with 0.05 mM PLP. Proteins were eluted with 3.5 mL of the same buffer. Total protein concentration was determined using the Pierce BCA total protein assay with bovine serum albumin (BSA) as the standard, per the manufacturer's instructions (Pierce Biotechnology, Inc., Rockford, Ill.). The resulting cell-free extract was used for subsequent assays.

For the tryptophan racemase assay a total of 650 μg of desalted protein was added for each enzyme based on Pierce BCA total protein analysis with BSA as the standard (Pierce Biotechnology, Inc., Rockford, Ill.). Formation of D-tryptophan was measured at 30 minutes, 2 hours, 4 hours and 24 hours. pSE420-cHis cell-free extract of the SEQ ID NO:412 polypeptide served as a positive control for the assay, and cell-free extract of empty vector pSE420-cHis served as a negative control.

TABLE 25
D-trp production, (pSE420-cHis constructs)
D-trp production, μg/mL

| Polypeptide (SEQ ID NO) | 30 min | 2 hours | 4 hours | 24 hours |
|---|---|---|---|---|
| pSE420-cHis vector control | nd | nd | nd | nd |
| 412 | 25 | 79 | 126 | 582 |
| 416 | 82 | 336 | 568 | 2344 |
| 418 | 59 | 209 | 307 | 1346 |
| 420 | nd | nd | nd | nd |
| 422 | nd | nd | nd | nd |
| 424 | 1407 | *no data | 3322 | 2564 |
| 426 | nd | nd | nd | nd |
| 428 | nd | nd | nd | nd |
| 430 | nd | nd | nd | nd |
| 432 | nd | nd | nd | nd |
| 434 | nd | nd | nd | nd |
| 436 | nd | nd | nd | nd |
| 438 | nd | nd | nd | nd |
| 440 | 864 | 2213 | 3022 | 3947 |

nd = not detected under the conditions of the assay as described above
*sample was not tested

Racemase polypeptides having the sequence shown in SEQ ID NO:420, 422, 426, 428, 430, 432, 434, 436, and 438 showed no detectable tryptophan racemase activity after 24 hours under the conditions tested. (Under the conditions described in Part A, good activity was observed for polypeptides having the sequence of SEQ ID NO:420, 422, 426, and 438; very slight activity was detected for polypeptides having the sequence of SEQ ID NO:428, 434, and 436; and no activity was detected for the polypeptide having the sequence of SEQ ID NO:440).

Racemase polypeptides having the sequence of SEQ ID NO:416, 418, 424 and 440 showed appreciable tryptophan activity in this assay. These were PCR amplified with and without C-terminal His tags for subcloning into pET30a. The oligonucleotides used for amplification are shown in Table 26.

TABLE 26
Oligonucleotide primers

| | | SEQ |
|---|---|---|
| | | ID |
| Designation | Sequence (5′ → 3′) | NO: |
| SEQ ID NO: 415- | GGCCTTAACTCGAGGCGGTTGATCTTCTTGGGGTTG | 506 |
| R Xho1 no stop | | |
| SEQ ID NO: 415- | GGCCTTAACATATGGCTCCCTATCTGCCCCTTGTGAC | 507 |
| F Nde1 | | |
| SEQ ID NO: 415- | CCTTGGAACTCGAGTTAGCGGTTGATCTTCTTGG | 508 |
| R Xho1 no stop | | |
| SEQ ID NO: 417- | GGCCTTAACATATGGCCCCTTACCTGCCGCTG | 509 |
| F Nde1 | | |
| SEQ ID NO: 417- | CCGGAACCTTGGAACCGTCGACTTAGCGTTTGATCTTCTTGGG | 510 |
| R Sal1 with stop | | |
| SEQ ID NO: 417- | GGCCTTAACCTTGTCGACGCGTTTGATCTTCTTGGGGTTGGTGTAG | 511 |
| R Sal1 no stop | | |
| SEQ ID NO: 423- | GGCCTTAACATATGGCTCCACCGCTGTCGATGGACAAC | 512 |
| F Nde1 | | |
| SEQ ID NO: 439- | GGCCTTAACATATGGCTAGCAATGCCTGGGTGGAGATAGAC | 513 |
| F Nde1 | | |
| SEQ ID NO: 439- | GGCCTTAACTCGAGTTAGGTGTTGCCCCAGACGGTGTAC | 514 |
| R Xho1 with stop | | |
| SEQ ID NO: 439- | GGCCTTAACTCGAGGGTGTTGCCCCAGACGGTGTACATGTCC | 515 |
| R Xho1 no stop | | |

Tagged and untagged constructs were sequenced for accuracy (Agencourt Bioscience Inc., Beverly Mass.) and transformed into BL21DE3; transformants were grown and induced in Novagen OvernightExpress AutoinductionSystem 2 (EMD Biosciences/Novagen catalog #71366) containing solutions 1-6 with the appropriate antibiotic selection, and cell-free extracts were prepared as described herein. Racemase candidate proteins were purified from tagged constructs and desalted on PD-10 columns. Untagged racemase candidate cell-free extracts were desalted on PD-10 columns. Protein concentrations were determined by Pierce BCA protein assay (Pierce Biotechnology, Inc., Rockford, Ill.) and racemase purity was estimated by Experion Automated Gel System (Experion, version A.01.10, Biorad, Hercules, Calif.).

Racemase assays were performed on purified and crude protein extracts as described in Example 17. Purified protein having the sequence shown in SEQ ID NO:412 served as a positive control. For the assay, 5 μg of equivalent BAR protein was added for the positive control, and an estimated 50 μg equivalent BAR protein was added for each of the other enzymes based on Pierce BCA total protein analysis and racemase purity estimation by Experion Automated Gel System (Experion, version A.01.10, Biorad, Hercules, Calif.).

TABLE 27
D-trp production
D-trp production, μg/mL

| Enzyme | 0.5 hr | 2 hr | 4 hr |
|---|---|---|---|
| SEQ ID NO: 412 purified | 2197 | 2431 | 3186 |
| SEQ ID NO: 416 purified | 52 | 54 | 134 |
| SEQ ID NO: 418 purified | 187 | 533 | 664 |
| SEQ ID NO: 424 purified | nd | nd | nd |
| SEQ ID NO: 440 purified | nd | nd | nd |
| SEQ ID NO: 416 CFE | nd | 84 | 100 |
| SEQ ID NO: 418 CFE | 36 | 82 | 74 |
| SEQ ID NO: 424 CFE | nd | nd | nd |
| SEQ ID NO: 440 CFE | nd | nd | nd |

nd = not detected under the conditions of the assay as described above;
4 candidates that appeared to have higher activity than the SEQ ID NO: 412 polypeptide - the SEQ ID NO: 416, 418, 424, and 440 polypeptides not replicated in pET30 with BL21DE3 host; all experiments conducted with purified protein (approx 50 μg BAR); previously shown in Part A that there are differences in activity when the same construct is in different host backgrounds.

Extracts of polypeptides having the sequence shown in SEQ ID NO:416, 418, 424 and 440 expressed in pSE420-cHis/TOP10 exhibited tryptophan racemase activity, while extracts from the same clones in pET30/BL21DE3 did not exhibit or exhibited very little tryptophan racemase activity. Polypeptides having the sequence shown in SEQ ID NO:424 and 440 showed no detectable tryptophan racemase activity in purified or crude cell extracts when cloned into pET30 and expressed in BL21DE3, under the conditions tested. Polypeptides having the sequence shown in SEQ ID NO:416 and 418 showed tryptophan racemase activity for both purified and crude extracts.

Since variations in racemase activity were observed with polypeptides having the sequence shown in SEQ ID NO:416, 418, 424 and 440 in different vector and host backgrounds, the reproducibility in the original pSE420-cHis vector was investigated. [It is noted that the SEQ ID NO:424 racemase candidate could not be revived from glycerol stocks.] Racemase assay under the conditions described earlier in this Example were was repeated using 1 mg total protein (from pSE420-cHis/TOP10 cell-free extracts) of polypeptides having the amino acid sequence shown in SEQ ID NO:416, 418 and 440. The 3 clones showed severely diminished racemase activity. Comparison of the racemase activity for polypeptides having SEQ ID NO:416, 418 and 440 show that inconsistent results were obtained despite using the same vector/host background. Conditions described under Part A resulted in similar observations of clone/construct instability of a few of the racemase candidates.

TABLE 28
D-trp production
D-trp production, μg/mL

| Enzyme | 0.5 hr | 2 hr | 4 hr |
|---|---|---|---|
| pSE420-cHis vector control | nd | nd | nd |
| SEQ ID NO: 412 | 636 | 1672 | 3154 |
| SEQ ID NO: 416 | 11 | nd | 78 |
| SEQ ID NO: 418 | 31 | 139 | 187 |
| SEQ ID NO: 440 | nd | 2 | nd |

nd = not detected under the conditions of the assay as described above

The host organisms, expression conditions, and post expression cell handling can all affect whether there is detectable tryptophan racemase activity under the conditions of the assay. Additionally, under optimized conditions, it is expected that all racemase candidates could have tryptophan racemase activity.

Racemase candidates were grouped by amino acid sequence homology, with clusters having 95% or greater homology at amino acid level to a reference sequence. One or more representatives was/were chosen from each group for characterization of tryptophan racemase activity under the conditions described in Part B.

Using SEQ ID NO:110 as the reference sequence, the following racemase candidates had 97% or greater identity at amino acid level to the above reference sequence: SEQ ID NO:136, 174, 138, and 296. SEQ ID NO:416 is a non-leadered version of the reference SEQ ID NO:110 sequence. Under the conditions described in Part B (see, for example, Example 17), tryptophan racemase activity was detected for the non-leadered version (SEQ ID NO:416) of the reference candidate, SEQ ID NO:110. Thus, it would be expected that other racemase candidates with 97% or greater sequence identity at the amino acid level would also have tryptophan racemase activity.

Using SEQ ID NO:116 as the reference sequence, the following racemase candidates had 97% or greater identity at amino acid level to the above reference sequence: SEQ ID NOs:150, 192, 152, 118, 194, 154, 196, 158, and 160. SEQ ID NO:420 is a non-leadered version of the reference SEQ ID NO:116 sequence. SEQ ID NO:422 is a non-leadered version of the reference SEQ ID NO:118 sequence. Under the conditions described in Part B (e.g., Example 17), tryptophan racemase activity was not detected for polypeptides having the amino acid sequence shown in SEQ ID NO:420 and 422, which are the non-leadered versions of SEQ ID NO:116 and 118, respectively. However, activity was observed for these polypeptides under the assay conditions described in Part A. The host organisms, expression conditions, and post-expression cell handling can all affect whether there is detectable tryptophan racemase activity under the conditions of the assay. Additionally, under optimized conditions or as shown in the assay conditions described in Part A, it is expected that all of the above racemase candidates could have tryptophan racemase activity.

It is expected that the presence of activity in a polypeptide encoded from a subcloned nucleic acid is predictive of the presence of activity in the corresponding polypeptide encoded from the full-length or wild type nucleic acid as indicated in Table 29.

TABLE 29

| Sub-clone number (SEQ ID NO) | Wild-Type Clone (SEQ ID NO) |
|---|---|
| 416 | 110 |
| 418 | 112 |
| 424 | 50 |
| 440 | 124 |

### Example 13

### Analysis of Racemases Provided as pSE420-cHis Clones

Nucleic acids having the sequence shown in SEQ ID NO:441, 443, 445, 447, 449, 451, and 453 (encoding racemase polypeptides having the sequence shown in SEQ ID NO:442, 444, 446, 448, 450, 452, and 454) were provided as pSE420-cHis clones. One skilled in the art can synthesize the genes encoding these racemases using various published techniques for example, as described in Stemmer et al., supra. The plasmids were transformed into TOP10-chemically competent cells (Invitrogen, Carlsbad, Calif.). Overnight cultures growing in LB carbenicillin (100 μg/ml) were diluted 100× in 50 ml LB carbenicillin in a 250 ml baffled flask. Cultures were grown at 30° C. and 250 rpm until they reached an OD₆₀₀ of 0.5 to 0.8, after which protein expression was induced with 1 mM IPTG for 4 h at 30° C. Samples for total protein were taken prior to induction and right before harvesting. Cells were harvested by centrifugation. Cells were frozen at −80° C.

Cell extracts were typically prepared from the above frozen pellets by adding 5 ml per g of cell pellet of Bugbuster Amine Free (Novagen/EMD Biosciences, San Diego, Calif.) with 5 μL/mL of Protease Inhibitor Cocktail II (Calbiochem, San Diego, Calif.) and 1 μl/ml of benzonase nuclease (Novagen/EMD Biosciences, San Diego, Calif.). Cell solutions were incubated at room temperature with gentle mixing for 15 min; cells were spun out at 14000 rpm for 20 min (at 4° C.) and the supernatant was carefully removed. Detergents and low molecular weight molecules were removed by passage through PD-10 columns (GE Healthcare, Piscataway, N.J.) previously equilibrated with 100 mM potassium phosphate (pH 7.8) with 0.05 mM PLP. Proteins were eluted with 3.5 mL of the same buffer. Total protein concentration was determined using the Pierce BCA protein assay (Pierce Biotechnology, Inc., Rockford, Ill.) with bovine serum albumin (BSA) as the standard, per the manufacturer's instructions. The resulting cell-free extract was used for subsequent assays.

Tryptophan racemase assays were carried out under the conditions described in Example 17. For the tryptophan racemization assay, a total of 1 mg of soluble protein (based on Pierce BCA total protein analysis with BSA as the standard) was added for each racemase candidate and positive controls. Cell free extract of polypeptides having the sequence shown in SEQ ID NO:412, pSE420/TOP10 construct served as positive control for the assay, and cell-free extract of TOP10 (Invitrogen, Carlsbad, Calif.) containing vector pSE420-cHis served as a negative control. Total protein concentration was determined using the Pierce BCA protein assay (Pierce Biotechnology, Inc., Rockford, Ill.) with bovine serum albumin (BSA) as the standard, per the manufacturer's instructions. Formation of D-tryptophan was measured at 30 minutes, 2 hours and 4 hours as described in Example 18.

TABLE 30
D-trp production
D-trp production, μg/mL

| Polypeptide (SEQ ID NO) | 0.5 hr | 2 hr | 4 hr |
|---|---|---|---|
| pSE420-cHis vector control | nd | nd | nd |
| 412 | 636 | 1672 | 3154 |
| 442 | 678 | 2107 | 3346 |
| 444 | 105 | 457 | 758 |
| 446 | 312 | 1549 | 1934 |
| 448 | 6 | 42 | nd |
| 450 | 23 | 66 | 33 |
| 452 | 32 | 36 | 85 |
| 454 | 257 | 577 | 888 |

nd = not detected under the conditions of the assay as described above

All of the racemase candidate extracts tested above, polypeptides having the sequence shown in SEQ ID NO:442, 444, 446, 448, 450, 452 and 454, had detectable tryptophan racemase activity under the conditions described above. In addition, tryptophan racemase activity was detected for the positive control, the SEQ ID NO:412 polypeptide extract, and there was no detectable activity in the case of the pSE420-cHis vector control extracts. It is expected that the homologs of the representative racemase candidates having 95% or greater homology at amino acid level (see Table 31) will also have tryptophan racemase activity.

TABLE 31

| Polypeptide (SEQ ID NO) | 0.5 hr | 2 hr | 4 hr |
|---|---|---|---|
| pSE420-cHis vector control | nd | nd | nd |
| 412 | 636 | 1672 | 3154 |
| 442 | 678 | 2107 | 3346 |
| 444 | 105 | 457 | 758 |
| 446 | 312 | 1549 | 1934 |
| 448 | 6 | 42 | nd |
| 450 | 23 | 66 | 33 |
| 452 | 32 | 36 | 85 |
| 454 | 257 | 577 | 888 |

nd, not detected under the conditions of the assay as described above

Racemase candidates described in this example were grouped by amino acid sequence homology with clusters having 95% or greater homology at amino acid level to a reference sequence. One or more representatives were chosen from each group for characterization of tryptophan racemase activity using the conditions described in Part B. Using SEQ ID NO:244 as the reference sequence, the following racemase candidates had 97% or greater identity at amino acid level to the above reference sequence: SEQ ID NO:248, 236, 246, 252, 250, and 254. SEQ ID NO:448 is a non-leadered version of the reference SEQ ID NO:244 sequence. Under the conditions described in Part B (e.g., Example 17), tryptophan racemase activity was detected for the non-leadered version (SEQ ID NO:448) of the reference candidate, SEQ ID NO:244; as well as the non-leadered version (SEQ ID NO:450) of the candidate, SEQ ID NO:248. Thus, it would be expected that other racemase candidates with 97% or greater sequence identity at the amino acid level would also have tryptophan racemase activity.

Using SEQ ID NO:288 as a reference sequence, the following racemase candidates had 97% or greater identity at amino acid level to the above reference sequence: SEQ ID NO:274, 234, 220, 222, 226, 232, 240, 242, 258, 260, 264, 266, 286, 290, 170, and 216. SEQ ID NO:454 is a non-leadered version of the reference SEQ ID NO:288 sequence; SEQ ID NO:452 is a non-leadered version of SEQ ID NO:274 sequence; and SEQ ID NO:446 is a non-leadered version of SEQ ID NO:234 sequence. Under the conditions of the assay as described in Example 17, tryptophan racemase activity was detected for the non-leadered version (SEQ ID NO:454) of the reference candidate, SEQ ID NO:288; as well as the non-leadered versions (SEQ ID NO:452 and 446) of racemase candidates SEQ ID NO:274 and 234, respectively. Thus, it would be expected that other racemase candidates listed above with 97% or greater sequence identity at the amino acid level would also have tryptophan racemase activity.

Using SEQ ID NO:218 as a reference sequence, the following racemase candidates had 97% or greater identity at amino acid level to the above reference sequence: SEQ ID NO:208, 210, 228, 230, 270, 272, 278, 280, 282, 284, 292, 198, 212, 214, and 114. SEQ ID NO:204 had 96% identity with SEQ ID NO:218 reference sequence. SEQ ID NO:444 is a non-leadered version of the reference SEQ ID NO:218 sequence. Under the conditions described in Part B (e.g., Example 17), tryptophan racemase activity was detected for the non-leadered version (SEQ ID NO:444) of the reference candidate, SEQ ID NO:218. Thus it would be expected that other racemase candidates with 97% or greater sequence identity at the amino acid level would also have tryptophan racemase activity.

SEQ ID NO:436 is a non-leadered version of SEQ ID NO:114 sequence. Under the conditions of the assays described in Part B, tryptophan racemase activity was not detected for the non-leadered version (SEQ ID NO:436) of the racemase candidate SEQ ID NO:114, as shown in Example 12.

The SEQ ID NO:441 Nucleic Acid (Encoding the Polypeptide Having the Sequence of SEQ ID NO:442) was Subcloned into pET30a with a C-Terminal His Tag

A D56N mutant (corresponding to D76N mutation in A. caviae) was created in SEQ ID NO:442. Mutagenesis was done using the QuickChange-Multi site-directed mutagenesis kit (Stratagene, La Jolla, Calif.), using the C-tagged SEQ ID NO:442 gene in pET30a as template. The following mutagenic primer was used to make the D56N change as described in Example 19: 5′-CGCCATCATGAAGGCGAACGCCTACGGTCACG-3′ (SEQ ID NO:516).

The site-directed mutagenesis was done as described in the manufacturer's protocol. The resulting mutation was detrimental to tryptophan racemase activity in this candidate, whereas, in A. caviae, the corresponding D76N mutation has a positive effect.

It is expected that the presence of activity in a polypeptide encoded from a subcloned nucleic acid is predictive of the presence of activity in the corresponding polypeptide encoded from the full-length or wild type nucleic acid as indicated in Table 32 below.

TABLE 32

| Subclone number (SEQ ID NO) | Wild type clone (SEQ ID NO) |
|---|---|
| 442 | 224 |
| 444 | 218 |
| 446 | 234 |
| 448 | 244 |
| 450 | 248 |
| 452 | 274 |
| 454 | 288 |

### Example 14

### Analysis of Racemases Provided as pSE420-cHis Clones

Nucleic acids having the sequence shown in SEQ ID NO:455, 457, 459, 461, 463, 465, 467, 469, 471, 473, 475, and 477 (encoding polypeptides having the sequence shown in SEQ ID NO:456, 458, 460, 462, 464, 466, 468, 470, 472, 474, 476, and 478) were provided as pSE420-cHis clones. One skilled in the art can synthesize the genes encoding these racemases using various published techniques for example, as described in Stemmer et al., supra. The plasmids were transformed into TOP10 chemically competent cells (Invitrogen, Carlsbad, Calif.). Overnight cultures growing in LB carbenicillin (100 μg/ml) were diluted 100× in 50 ml LB carbenicillin (100 μg/ml) in a 250 ml baffled flask. Cultures were grown at 30° C. at 250 rpm until they reached an OD₆₀₀ of 0.5 to 0.8, after which protein expression was induced with 1 mM IPTG for 4 h at 30° C. Samples for total protein were taken prior to induction and right before harvesting. Cells were harvested by centrifugation and frozen at −80° C.

Cell extracts were typically prepared from the above frozen pellets by adding 5 ml per g of cell pellet of Bugbuster Amine Free (Novagen/EMD Biosciences, San Diego, Calif.) with 5 μL/mL of Protease Inhibitor Cocktail II (Calbiochem, San Diego, Calif.) and 1 μl/ml of benzonase nuclease (Novagen/EMD Biosciences, San Diego, Calif.). Cell solutions were incubated at room temperature with gentle mixing for 15 min; cells were spun out at 14,000 rpm for 20 min (at 4° C.) and the supernatant was carefully removed. Detergents and low molecular weight molecules were removed by passage through PD-10 columns (GE Healthcare, Piscataway, N.J.) previously equilibrated with 100 mM potassium phosphate (pH 7.8) with 0.05 mM PLP. Proteins were eluted with 3.5 mL of the same buffer. Total protein concentration was determined using the Pierce BCA (Pierce Biotechnology, Inc., Rockford, Ill.) protein assay with bovine serum albumin (BSA) as the standard, per the manufacturer's instructions. The resulting cell-free extract was used for subsequent assays.

Desalted cell-free extracts of racemase polypeptides having the sequence of SEQ ID NO:456, 458, 460, 462, 464, 466, 468, 470, 472, 474, 476, 478 were prepared as described in other examples.

Tryptophan racemase assays were carried out under the conditions described in Example 17. For the tryptophan racemization assay a total of 800 μg of soluble protein was added for each racemase candidate and positive controls. pSE420-cHis/TOP10 cell-free extracts of racemase polypeptides having the sequence shown in SEQ ID NO:412 and 442 served as positive controls for the assay, and cell-free extract of TOP10 (Invitrogen, Carlsbad, Calif.) containing vector pSE420-cHis served as a negative control. Total protein concentration was determined using the Pierce BCA (Pierce Biotechnology, Inc., Rockford, Ill.) protein assay with bovine serum albumin (BSA) as the standard, per the manufacturer's instructions. Formation of D-tryptophan was measured at 30 minutes, 2 hours and 4 hours as described in Example 18.

TABLE 33
D-trp production
D-trp production, μg/mL

| Polypeptides (SEQ ID NO) | 0.5 hr | 2 hr | 4 hr |
|---|---|---|---|
| pSE420-cHis | nd | nd | nd |
| 412 | 834 | 1861 | 2803 |
| 442 | 911 | 1863 | 2912 |
| 456 | nd | nd | nd |
| 458 | nd | nd | nd |
| 460 | 11 | 5 | 105 |
| 462 | nd | nd | nd |
| 464 | nd | nd | nd |
| 466 | nd | nd | nd |
| 468 | nd | nd | nd |
| 470 | nd | nd | nd |
| 472 | nd | nd | nd |
| 474 | 121 | 455 | 1101 |
| 476 | 28 | 100 | 249 |
| 478 | nd | nd | nd |

nd = not detected under the conditions of the assay as described.

Racemase polypeptides having the sequence shown in SEQ ID NO:460, 474 and 476 showed tryptophan racemase activity. Racemase polypeptides having the sequence shown in SEQ ID NO:456, 458, 462, 464, 466, 468, 470, 472 and 478 showed no detectable tryptophan racemase activity after 4 hours under the conditions tested. In a follow up experiment, a 24-hour sample was evaluated for D-tryptophan production. None of the racemases listed above showed detectable tryptophan racemase activity at 24 hours under the conditions described above. Of the candidates for which no activity was observed, racemase polypeptides having the sequence shown in SEQ ID NO:456, 458, 462, 464, 466, 468, 470, 472 and 478 exhibited poor or questionable soluble protein expression. The host organisms, expression conditions, and post expression cell handling can all affect whether there is detectable tryptophan racemase activity under the conditions of the assay. Additionally, under optimized conditions, it is expected that all racemase candidates could have tryptophan racemase activity.

Racemase candidates were grouped by amino acid sequence homology, with clusters having 95% or greater homology at amino acid level to a reference sequence. One or more representatives was/were chosen from each group for characterization of tryptophan racemase activity using the conditions described in Part B.

Using SEQ ID NO:108 as the reference sequence, the following racemase candidates had 96% or greater identity at amino acid level to the above reference sequence: SEQ ID NO:172, 178, 180, 182, 184, 140, 144, 188, 190, 112, 148, 156, 120 and 162. SEQ ID NO:474 is a non-leadered version of the reference SEQ ID NO:108 sequence. Under the conditions described in Part B (e.g., Example 17), tryptophan racemase activity was detected for the non-leadered version (SEQ ID NO:474) of the reference candidate, SEQ ID NO:108, as well as the non-leadered version (SEQ ID NO:460) of SEQ ID NO:120 which is 97% identical with the reference candidate, SEQ ID NO:108. Additionally the non-leadered version (SEQ ID NO:418) of SEQ ID NO:112 was shown to have detectable tryptophan racemase activity as seen in Example 12. Thus it would be expected that the other racemase candidates listed above, with 96% or greater sequence identity at the amino acid level would also have tryptophan racemase activity.

It is expected that the presence of activity in a polypeptide encoded from a subcloned nucleic acid is predictive of the presence of activity in the corresponding polypeptide encoded from the full-length or wild type nucleic acid.

TABLE 34

| Sub-clone number (SEQ ID NO) | Wild-Type Clone (SEQ ID NO) |
|---|---|
| 459 | 119 |
| 473 | 107 |
| 475 | 299 |

### Example 15

### Analysis of Racemase Candidates Provided as PCR Products First Group

Racemase nucleic acid sequences SEQ ID NO:313, 325, 341, 343, 317, 329, 327, 345, 333, and 351 were provided as PCR products with NdeI and NotI restriction sites at the 5′ and 3′ ends, respectively. The PCR fragments were cloned into pCR-Blunt II-Topo (Invitrogen, Carlsbad, Calif.) as recommended by the manufacturer. The sequence was verified by sequencing (Agencourt, Beverly, Mass.) and an insert with the correct sequence was then released from the vector using NdeI and NotI restriction enzymes and ligated into the NdeI and NotI restriction sites of pET30a. One skilled in the art can synthesize the genes encoding these racemases using various published techniques for example, as described in Stemmer et al., supra.

The pET30a constructs of all racemase candidates listed above were transformed into the expression host BL21DE3. Liquid cultures were grown overnight in LB medium (BD Diagnostics, Franklin Lakes, N.J.) containing 50 μg/ml kanamycin at 37° C. with agitation at 250 rpm. These overnight cultures were used to inoculate shake flasks containing 50 mL Overnight Express™ media (Solutions 1-6, Novagen/EMD Biosciences, San Diego, Calif.) containing 50 μg/ml kanamycin. Overnight Express™ cultures were grown at 30° C. with agitation at 250 rpm for approximately 20 hours, and cells were harvested by centrifugation when OD₆₀₀ reached ˜6-10.

Cell extracts were typically prepared from the above frozen pellets by adding 5 ml per g of cell pellet of Bugbuster Amine Free (Novagen/EMD Biosciences, San Diego, Calif.) with 5 μL/mL of Protease Inhibitor Cocktail II (Calbiochem, San Diego, Calif.) and 1 μl/ml of benzonase nuclease (Novagen/EMD Biosciences, San Diego, Calif.). Cell solutions were incubated at room temperature with gentle mixing for 15 min; cells were spun out at 14000 rpm for 20 min (at 4° C.) and the supernatant was carefully removed. Detergents and low molecular weight molecules were removed by passage through PD-10 columns (GE Healthcare, Piscataway, N.J.) previously equilibrated with 100 mM potassium phosphate (pH 7.8) with 0.05 mM PLP. Proteins were eluted with 3.5 mL of the same buffer. Total protein concentration was determined using the Pierce BCA protein assay with bovine serum albumin (BSA) as the standard, per the manufacturer's instructions (Pierce Biotechnology, Inc., Rockford, Ill.). The resulting cell-free extract was used for subsequent assays.

Desalted cell-free extracts were evaluated using tryptophan racemase assays under the conditions described in Example 17, with purified SEQ ID NO:442 polypeptides serving as a positive control. For the tryptophan racemase assay, a total of 10 μg and 100 μg BAR-equivalent SEQ ID NO:442 racemase (based on Pierce BCA total protein analysis with BSA as the standard and estimation of percentage of BAR protein expressed from Experion, (Experion, version A.01.10, Biorad, Hercules, Calif.)), were used as positive controls. 1 mg of total protein was added for each racemase candidate being tested (based on Pierce BCA total protein analysis with BSA as the standard). Formation of D-tryptophan was measured at 30 minutes, 1 hour, 2 hours, and 4 hours as described in Example 18. In a follow up experiment, a 24-hour sample was evaluated for D-tryptophan production.

None of the racemases listed above showed detectable tryptophan racemase activity at 24 hours under the conditions described herein. Tryptophan racemase activity was seen for positive control, SEQ ID NO:442. The host organisms, expression conditions, and post expression cell handling can all affect whether there is detectable tryptophan racemase activity under the conditions of the assay. Additionally, under optimized conditions, it is expected that all racemase candidates could have tryptophan racemase activity.

### Second Group

Racemase nucleic acids SEQ ID NO:321, 323 and 347 were provided as PCR products with Nde I and Not I restriction sites at the 5′ and 3′ ends, respectively. However all of these sequences had additional Nde I and/or Not I sites internal to the gene sequence so direct subcloning was not possible. SEQ ID NO:349 was re-amplified by PCR with RTth polymerase (Applied Biosystems, Foster City, Calif.) and primers adding an Nde I and Xho I restriction site at the 5′ and 3′ ends, respectively.

TABLE 35

| Designation | Sequence (5′ → 3′) | SEQ ID NO: |
|---|---|---|
| SEQ ID | TAAGAAGGAGATATACATATGGAATTCGATTGGATTCG | 517 |
| NO: 323 | | |
| infusion F | | |
| Nde1 | | |
| SEQ ID | GGTGGTGGTGCTCGAGTGCGGCCGCTTATAACACCTG | 518 |
| NO: 323 | | |
| infusion R | | |
| Not1 | | |
| SEQ ID | TAAGAAGGAGATATACATATGTCGCATTCCACCACCTGG | 519 |
| NO: 347 | | |
| infusion F | | |
| Nde1 | | |
| SEQ ID | GGTGGTGGTGCTCGAGTGCGGCCGCTCAGCGATACTG | 520 |
| NO: 347 | | |
| infusion R | | |
| Not1 | | |
| SEQ ID | TAAGAAGGAGATATACATATGAAAAGTGCAGGCATTATA G | 521 |
| NO: 321 | | |
| infusion F | | |
| Nde1 | | |
| SEQ ID | GGTGGTGGTGCTCGAGTGCGGCCGCTTAAGCCTTAGT | 522 |
| NO: 321 | | |
| infusion R | | |
| Not1 | | |

The PCR fragment was digested with NdeI and XhoI restriction enzymes and ligated into the NdeI and XhoI restriction sites of pET30a. Correct plasmids were verified by digestion with NdeI and XhoI and sequencing (Agencourt, Beverly, Mass.). One skilled in the art can synthesize the genes encoding these racemases using various published techniques for example, as described in Stemmer et al., supra.

The pET30a clones of all of the above racemases were transformed into expression host BL21DE3. Liquid cultures were grown overnight (LB kanamycin 50, 37° C., 250 rpm) and used to inoculate shake flasks containing 50 mL Overnight Express™ media (Solutions 1-6, Novagen/EMD Biosciences, San Diego, Calif.) containing kanamycin. Overnight Express™ cultures were grown at 30° C. and 250 rpm for approximately 20 hours, and collected when the OD₆₀₀ reached ˜6-10. Cells were harvested by centrifugation.

Desalted cell-free extracts of racemase polypeptides SEQ ID NO:322, 324, and 348 were prepared as described above.

Tryptophan racemase assays were carried out under the conditions described in Example 17, with purified A. caviae D76N BAR (see Example 19) serving as a positive control. For the tryptophan racemase assay, a total of 50 μg BAR equivalent of positive control (based on Pierce BCA total protein analysis with BSA as the standard and estimation of percentage of BAR protein expressed from Experion (Experion, version A.01.10, Biorad, Hercules, Calif.) was added. 1 mg of total protein was added for each racemase candidate being tested (based on Pierce BCA total protein analysis with BSA as the standard). Formation of D-tryptophan was measured at 1 hour, 2 hours, 4 hours, and 21.5 hours as described in Example 18.

TABLE 36
D-trp production
D-trp production, μg/mL

| Polypeptide (SEQ ID NO) | 1 hour | 2 hours | 4 hours | 21.5 hours |
|---|---|---|---|---|
| A. caviae D76N pure | 742.7 | 1368 | 2160.7 | 2458.7 |
| 322 | 305 | 437.7 | 596 | 1174.3 |
| 324 | nd | nd | nd | nd |
| 348 | nd | nd | nd | nd |

nd = not detected under the conditions of the assay as described above.

Tryptophan racemase activity was observed for polypeptides having the sequence shown in SEQ ID NO:322. This enzyme is interesting because it is the smallest racemase protein that was active on tryptophan, with the protein being only 232 amino acids (as compared to 409 amino acids for the A. caviae benchmark, and >300 amino acids for most of the other racemase candidates).

There was no detectable tryptophan racemase activity observed for polypeptides having the sequence shown in SEQ ID NO:324 and 348 under the conditions tested. SDS-PAGE analysis showed good soluble protein expression for the SEQ ID NO:348 polypeptide, but minimal soluble protein expression for the SEQ ID NO:324 polypeptide. The host organisms, expression conditions, and post expression cell handling can all affect whether there is detectable tryptophan racemase activity under the conditions of the assay. Additionally, under optimized conditions, it is expected that all racemase candidates could have tryptophan racemase activity.

### Third Group

Racemase nucleic acids having the sequence of SEQ ID NO:339 and 349 (encoding polypeptides having the sequence of SEQ ID NO:340 and 350, respectively) were provided as PCR products with NdeI and NotI restriction sites at the 5′ and 3′ ends, respectively. However, all of these sequences had additional NdeI and/or NotI sites internal to the gene sequence so direct subcloning was not carried out. The nucleic acid having the sequence of SEQ ID NO:350 was re-amplified by PCR with RTth polymerase (Applied Biosystems, Foster City, Calif.) and primers adding an NdeI and XhoI restriction site at the 5′ and 3′ ends, respectively.

TABLE 37

| | | SEQ ID |
|---|---|---|
| Designation | Sequences (5′ → 3′) | NO: |
| SEQ ID NO349-FNde1 | GGTTCCAACATATGACCGGGCTGACGGTAACGGCGGTG | 523 |
| SEQ ID NO: 349-RXho1 | GGTTCCAACTCGAGATAATAGCGGCCGCTCATCCC | 524 |
| SEQ ID NO: 339- | TAAGAAGGAGATATACATATGAGACCAGCTCGCGTTAGC | 525 |
| infusion F Nde1 | | |
| SEQ ID NO: 339 | GGTGGTGGTGCTCGAGTGCGGCCGCCTAGTTCCAGTT | 526 |
| infusion R Not1 | | |

The PCR fragment was digested with Nde I and Xho I restriction enzymes and ligated into the NdeI and XhoI restriction sites of pET30a. Correct plasmids were verified by digestion with NdeI and XhoI and sequencing (Agencourt, Beverly, Mass.). One skilled in the art can synthesize the genes encoding these racemases using various published techniques for example, as described in Stemmer et al., supra.

The pET30a constructs of all racemase candidates listed above were transformed into the expression host BL21DE3. Liquid cultures were grown overnight in LB medium (BD Diagnostics, Franklin Lakes, N.J.) containing 50 μg/ml kanamycin at 37° C. with agitation at 250 rpm. These overnight cultures were used to inoculate shake flasks containing 50 mL Overnight Express™ media (Solutions 1-6, Novagen/EMD Biosciences, San Diego, Calif.) containing 50 μg/ml kanamycin. Overnight Express™ cultures were grown at 30° C., with agitation at 250 rpm for approximately 20 hours, and cells were harvested by centrifugation when the OD₆₀₀ₙₘ reached between 6 and 10.

Desalted cell-free extracts of racemase polypeptides having the sequence of SEQ ID NO:340 and 350 were prepared as described above.

Tryptophan racemase assays were carried out under the conditions described in Example 17, with the polypeptide having the sequence of SEQ ID NO:412 serving as a positive control.

For the tryptophan racemase assay, a total of approximately 5 μg BAR equivalent of control (based on Pierce BCA total protein analysis with BSA as the standard and estimation of percentage of BAR protein expression level from Experion, (Experion, version A.01.10, Biorad, Hercules, Calif.)) was added, and 1 mg of total cell-free protein extract was added for each racemase candidate being tested (based on Pierce BCA total protein analysis with BSA as the standard). Formation of D-tryptophan was measured at 15 minutes, 2 hours, and 21 hours as described in Example 18.

No tryptophan racemization was detected for polypeptides having the sequence of SEQ ID NO:340 or 350 under the conditions tested. Positive control polypeptides having the sequence of SEQ ID NO:412 showed tryptophan racemase activity. SDS-PAGE analysis showed low soluble protein expression for SEQ ID NO:340 and 350 polypeptides. The host organisms, expression conditions, and post expression cell handling can all affect whether there is detectable tryptophan racemase activity under the conditions of the assay. Additionally, under optimized conditions, it is expected that all racemase candidates could have tryptophan racemase activity.

### Example 16

### Analysis of Racemases Provided as PCR-4-Blunt TOPO Clones

Racemase nucleic acids having the sequence shown in SEQ ID NO:335, 337, 357, 359, 361, and 365 were provided as PCR-4-Blunt TOPO clones. Racemases in these plasmids were amplified with RTth polymerase (Applied Biosystems, Foster City, Calif.) and primers adding an NdeI and XhoI restriction site at the 5′ and 3′ ends, respectively.

TABLE 38

| | | SEQ ID |
|---|---|---|
| Clone | Sequence (5′ → 3′) | NO: |
| SEQ ID NO: 335-F- | GGTTCCAAGGCATATGAGAACGAGACATCGATGCTG | 527 |
| Nde1 | | |
| SEQ ID NO: 334-R- | GGTTCCAATTCTCGAGTCAGGCTGCGTGACTGCCGCGA | 528 |
| Xho1 | | |
| SEQ ID NO: 337-F- | TTCCAATTGGCATATGCACCGCGTCTGGGCTGAAATC | 529 |
| Nde1 | | |
| SEQ ID NO: 337-R- | GGTTCCAACTCGAGTCACACATACACACGCGCCACGC | 530 |
| Xho1 | | |
| SEQ ID NO: 355-F- | TTGGAACCTTCATATGCAAACGAAACCCGCGCCCCG | 531 |
| Nde1 | | |
| SEQ ID NO: 355-R- | GGTTCCAAGGCTCGAGTTAGCGAATGTAAACCCGTTCCAC | 532 |
| Xho1 | | |
| SEQ ID NO: 357-F- | CCTTGGAACATATGGAACGAATCGTCCAGAAGCTGC | 533 |
| Nde1 * | | |
| SEQ ID NO: 357-R- | GGCCTTAACTCGAGTTATGACATCCGCGGAATCC | 534 |
| Xho1 * | | |
| SEQ ID NO: 359-F- | CCTTGGAACATATGGAACGAATCGTCCAGAAGCTGC | 535 |
| Nde1 * | | |
| SEQ ID NO: 359-R- | GGCCTTAACTCGAGTTATGACATCCGCGGAATCC | 536 |
| Xho1 * | | |
| SEQ ID NO: 361-F- | CCTTGGAACATATGGAACGAATCGTCCAGAAGCTGC | 537 |
| Nde1 * | | |
| SEQ ID NO: 361-R- | GGCCTTAACTCGAGTTATGACATCCGCGGAATCC | 538 |
| Xho1 * | | |
| SEQ ID NO: 365-F- | CCTTGGAACATATGGAACGAATCGTCCAGAAGCTGC | 539 |
| Nde1 * | | |
| SEQ ID NO: 365-R- | GGCCTTAACTCGAGTTATGACATCCGCGGAATCC | 540 |
| Xho1 * | | |

* Same forward and reverse primer pair was used for SEQ ID NO: 357, 359, 361, and 365 due to 100% DNA homology in primer regions.

The PCR fragments were cloned into pCR-Blunt II-Topo (Invitrogen, Carlsbad, Calif.) as recommended by the manufacturer. The sequence was verified by sequencing (Agencourt, Beverly, Mass.) and an insert with the correct sequence was then released from the vector using NdeI and XhoI restriction enzymes (New England Biolabs, Ipswich, Mass.) and ligated into the NdeI and XhoI restriction sites of pET30a. See Table above for specific primers and plasmids names. (It is noted that the TOPO cloning efforts for the SEQ ID NO:355 nucleic acid were unsuccessful after multiple attempts, so this racemase was not further processed). One skilled in the art can synthesize the genes encoding these racemases using various published techniques for example, as described in Stemmer et al., supra.

The pET30a constructs of all racemase candidates listed above were transformed into the expression host BL21DE3. Liquid cultures were grown overnight in LB medium (BD Diagnostics, Franklin Lakes, N.J.) containing 50 μg/ml kanamycin at 37° C. with agitation at 250 rpm. These overnight cultures were used to inoculate shake flasks containing 50 mL Overnight Express™ media (Solutions 1-6, Novagen/EMD Biosciences, San Diego, Calif.) containing 50 μg/ml kanamycin. Overnight Express™ cultures were grown at 30° C. with agitation at 250 rpm for approximately 20 hours, and cells were harvested by centrifugation when OD₆₀₀ reached ˜6-10.

Desalted cell-free extracts of racemase polypeptides having SEQ ID NO:336, 338, 358, 360, 362, and 366 were prepared as described below (polypeptides having SEQ ID NO:356 and 364 from this experiment were not further characterized).

Cell extracts were typically prepared from the above frozen pellets by adding 5 ml per g of cell pellet of Bugbuster Amine Free (Novagen/EMD Biosciences, San Diego, Calif.) with 5 μL/mL of Protease Inhibitor Cocktail II (Calbiochem, San Diego, Calif.) and 1 μl/ml of benzonase nuclease (Novagen/EMD Biosciences, San Diego, Calif.). Cell solutions were incubated at room temperature with gentle mixing for 15 min; cells were spun out at 14000 rpm for 20 min (at 4° C.) and the supernatant was carefully removed. Detergents and low molecular weight molecules were removed by passage through PD-10 columns (GE Healthcare, Piscataway, N.J.) previously equilibrated with 100 mM potassium phosphate (pH 7.8) with 0.05 mM PLP. Proteins were eluted with 3.5 mL of the same buffer. Total protein concentration was determined using the Pierce BCA (Pierce Biotechnology, Inc., Rockford, Ill.) protein assay with bovine serum albumin (BSA) as the standard, per the manufacturer's instructions. The resulting cell-free extract was used for subsequent assays.

Tryptophan racemase assays were carried out under the conditions described in Example 17, with purified A. caviae D76N BAR (Example 19) serving as a positive control. A total of 100 μg BAR equivalent of control was added (based on Pierce BCA total protein analysis with BSA as the standard and estimation of percentage of BAR protein expressed from Experion, version A.01.10, Biorad, Hercules, Calif.), and 1 mg of total protein was added for each racemase candidate being tested. Formation of D-tryptophan was measured at 30 minutes, 2 hours, 4 hours and 52 hours as described in Example 18.

TABLE 39
D-trp production
D-tryptophan production, μg/mL

| | 30 | | | |
|---|---|---|---|---|
| Polypeptide (SEQ ID NO) | min | 2 hours | 4 hours | 52 hours |
| 336 | 1485 | 2787 | 2705 | 2434 |
| 338 | 1503 | 2757 | 2738 | 2353 |
| 366 | nd | nd | nd | nd |
| 358 | nd | nd | nd | 27 |
| 360 | nd | nd | nd | nd |
| 362 | nd | nd | nd | nd |
| 442 purified - 100 μg | 340 | 1020 | 1726 | 3011 |
| A. caviae D76N purified - 100 μg | 1036 | 2804 | 2849 | 3011 |

nd = not detected under the conditions of the assay as described above.

Racemase polypeptides having SEQ ID NO:336, 338 and 358 were active. Racemase polypeptides having SEQ ID NO:366, 360, and 362 showed no detectable tryptophan racemase activity under the conditions tested. Polypeptides having SEQ ID NO:366, 360, and 362 all had satisfactory soluble protein expression. The host organisms, expression conditions, and post expression cell handling can all affect whether there is detectable tryptophan racemase activity under the conditions of the assay. Additionally, under optimized conditions, it is expected that all racemase candidates could have tryptophan racemase activity.

### Example 17

### Description of Racemase Assay Conditions

Leucine, Phenylalanine, Tryptophan, Methionine, Tyrosine, Alanine, Lysine, Aspartic Acid, Glutamate Racemase Assay

Racemase assays were performed starting with the L-amino acid isomer and the formation of corresponding D-amino acid was followed.

Assay conditions:

30 mM L-amino acid (L-Leucine, L-Phenylalanine, L-Tryptophan, L-Methionine, L-Tyrosine, L-Alanine, L-Lysine, L-Aspartic Acid, or L-Glutamate), 50 mM Potassium phosphate buffer (pH 8.0), 0.05 mM PLP, and water was added to make the volume up to 1 mL.

The assays were conducted at 30° C. with shaking at 225 rpm. Desalted racemase candidate proteins (cell-free extracts or purified preparations) were evaluated for amino acid racemase activity. Wherever possible, appropriate negative and positive controls were included for the assays. Sample aliquots were taken for analysis at various timepoints and formic acid was added to a final concentration of 2% to stop the reaction. Samples were frozen at −80° C., then thawed, centrifuged and filtered through 0.2μ filter (Pall Life Sciences, Ann Arbor, Mich.). Samples were analyzed for D-amino acid using the chiral LC/MS/MS method described in Example 18.

Monatin Racemase Assay

A subset of racemase candidates that gave promising tryptophan racemase results was tested for monatin racemization.

Assay conditions:

10 mM R,R monatin, 50 mM Potassium phosphate buffer (pH 8.0), 0.05 mM PLP, and water were added to make the volume up to 1 mL.

The assays were performed at 30° C. with shaking at 225 rpm. At various time points, sample aliquots were taken, diluted five-fold with distilled water, then filtered through a 0.2μ filter (Pall Life Sciences, Ann Arbor, Mich.) and stored at −80° C. for subsequent analysis. Samples were analyzed for the distribution of monatin stereoisomers as described in Example 18.

### Example 18

### Detection of Monatin Stereoisomers and Chiral Detection of Lysine, Alanine, Methionine, Tyrosine, Leucine, Phenylalanine, Tryptophan, Glutamate, and Aspartate

This example describes methods used to detect the presence of stereoisomers of monatin, lysine, alanine, methionine, tyrosine, leucine, phenylalanine, tryptophan, glutamate, and aspartate. It also describes a method for the separation and detection of the four stereoisomers of monatin.

### Chiral LC/MS/MS (“MRM”) Measurement of Monatin

Determination of the stereoisomer distribution of monatin in in vitro reactions was accomplished by derivatization with 1-fluoro-2-4-dinitrophenyl-5-L-alanine amide (“FDAA”), followed by reversed-phase LC/MS/MS MRM measurement.

Derivatization of Monatin with FDAA

To 50 μL of sample or standard and 10 μL of internal standard was added 100 μL of a 1% solution of FDAA in acetone. Twenty μL of 1.0 M sodium bicarbonate was added, and the mixture incubated for 1 h at 40° C. with occasional mixing. The sample was removed and cooled, and neutralized with 20 μL of 2.0 M HCl (more HCl may be required to effect neutralization of a buffered biological mixture). After degassing was complete, samples were ready for analysis by LC/MS/MS.

### LC/MS/MS Multiple Reaction Monitoring for the Determination of the Stereoisomer Distribution of Monatin

Analyses were performed using the LC/MS/MS instrumentation described above. LC separations capable of separating all four stereoisomers of monatin (specifically FDAA-monatin) were performed on a Phenomenex Luna 2.0×250 mm (3 μm) C18 (2) reversed phase chromatography column at 40° C. The LC mobile phase consisted of A) water containing 0.05% (mass/volume) ammonium acetate and B) acetonitrile. The elution was isocratic at 13% B, 0-2 min, linear from 13% B to 30% B, 2-15 min, linear from 30% B to 80% B, 15-16 min, isocratic at 80% B 16-21 min, and linear from 80% B to 13% B, 21-22 min, with an 8 min re-equilibration period between runs. The flow rate was 0.23 mL/min, and PDA absorbance was monitored from 200 nm to 400 nm. All parameters of the ESI-MS were optimized and selected based on generation of deprotonated molecular ions ([M-H]—) of FDAA-monatin, and production of characteristic fragment ions.

The following instrumental parameters were used for LC/MS analysis of monatin in the negative ion ESI/MS mode: Capillary: 3.0 kV; Cone: 40 V; Hex 1: 15 V; Aperture: 0.1 V; Hex 2: 0.1 V; Source temperature: 120° C.; Desolvation temperature: 350° C.; Desolvation gas: 662 L/h; Cone gas: 42 L/h; Low mass resolution (Q1): 14.0; High mass resolution (Q1): 15.0; Ion energy: 0.5; Entrance: OV; Collision Energy: 20; Exit: OV; Low mass resolution (Q2): 15; High mass resolution (Q2): 14; Ion energy (Q2): 2.0; Multiplier: 650. Three FDAA-monatin-specific parent-to daughter transitions are used to specifically detect FDAA-monatin in in vitro and in vivo reactions. The transitions monitored for monatin are 542.97 to 267.94, 542.97 to 499.07, and 542.97 to 525.04. Monatin internal standard derivative mass transition monitored was 548.2 to 530.2. Identification of FDAA-monatin stereoisomers is based on chromatographic retention time as compared to purified synthetic monatin stereoisomers, and mass spectral data. An internal standard was used to monitor the progress of the reaction and for confirmation of retention time of the S,S stereoisomer.

### Detection of L- and D-Amino Acids by LC/MS/MS

Samples containing a mixture of L- and D-amino acids such as lysine, alanine, methionine, tyrosine, leucine, phenylalanine, tryptophan, glutamate, and aspartate from biochemical reaction experiments were first treated with formic acid to denature protein. The sample was then centrifuged and filtered through a 0.2 μm nylon syringe filter prior to LC/MS/MS analysis. Identification of L- and D-amino acids was based on retention time and mass selective detection. LC separation was accomplished by using Waters 2690 liquid chromatography system and an ASTEC 2.1 mm×250 mm Chirobiotic TAG chromatography column with column temperature set at 45° C. LC mobile phase A and B were 0.25% acetic acid and 0.25% acetic acid in methanol, respectively. Isocratic elution was used for all methods to separate the L and D isomers. Lysine was eluted using 80% mobile phase A, and 20% B and a flow rate of 0.25 mL/min. Glutamate, alanine, and methionine were separated with elution of 60% mobile phase A and 40% B and a flow rate of 0.25 mL/min. Aspartate, tryptophan, tyrosine, leucine, and phenylalanine were separated isomerically with 30% mobile phase A and 70% B with a flow rate of 0.3 mL/min for aspartate and tryptophan, and 0.25 mL/min for tyrosine, leucine, and phenylalanine.

The detection system for analysis of L- and D-amino acids included a Waters 996 Photo-Diode Array (PDA) detector and a Micromass Quattro Ultima triple quadrupole mass spectrometer. The PDA, scanning from 195 to 350 nm, was placed in series between the chromatography system and the mass spectrometer. Parameters for the Micromass Quattro Ultima triple quadrupole mass spectrometer operating in positive electrospray ionization mode (+ESI) were set as the following: Capillary: 3.2 kV; Cone: 20 V; Hex 1: 12 V; Aperture: 0.1 V; Hex 2: 0.2V; Source temperature: 120° C.; Desolvation temperature: 350° C.; Desolvation gas: 641 L/h; Cone gas: 39 L/h; Low mass Q1 resolution: 16.0; High mass Q1 resolution: 16.0; Ion energy 1: 0.1; Entrance: −5; Collision: 20; Exit 1: 10; Low mass Q2 resolution: 16.0; High mass Q2 resolution: 16.0 Ion energy 2: 1.0; Multiplier: 650 V. MS/MS experiments with Multiple Reaction Monitoring (MRM) mode were set up to selectively monitor reaction transitions of 147.8 to 84.03, 147.8 to 56.3, and 147.8 to 102.2 for glutamate, 133.85 to 74.03, 133.85 to 69.94 and 133.85 to 87.99 for aspartate, 146.89 to 84.09, 146.89 to 55.97 and 146.89 to 67.23 for lysine, 149.80 to 56.1, 149.8 to 61.01, and 149.80 to 104.15 for methionine, 181.95 to 135.97, 181.95 to 90.88 and 181.95 to 118.87 for tyrosine, 131.81 to 86.04 and 131.81 to 69.31 for leucine, 90.0 to 44.3 for alanine, and 165.83 to 102.96, 165.83 to 93.27 and 165.83 to 120.06 for phenylalanine. In the case where numerous transitions are listed, the first transition listed was used for quantification. For tryptophan, MS/MS experiments with Multiple Reaction Monitoring (MRM) mode were set up to selectively monitor reaction transitions of 205.0 to 145.91, 205.0 to 117.92, and 205.0 to 188.05, and the transition from 212.0 to 150.98 for d8-DL tryptophan. Tryptophan quantification was achieved by determining the ratio of analyte response of transition 205.0 to 145.91 to that of the internal standard, d8-D,L tryptophan.

### Production of Monatin for Standards and for Assays

A racemic mixture of R,R and S,S monatin was synthetically produced as described in U.S. Pat. No. 5,128,482.

The R,R and S,S monatin were separated by a derivatization and hydrolysis step. Briefly, the monatin racemic mixture was esterified, the free amino group was blocked with Cbz, a lactone was formed, and the S,S lactone was selectively hydrolyzed using an immobilized protease enzyme. The monatin can also be separated as described in Bassoli et al., 2005, Eur. J. Org. Chem., 8:1652-1658.

### Example 19

### Cloning and Analysis of Broad Activity Racemase (BAR) from Aeromonas caviae ATCC 14486

This example describes the cloning of the A. caviae BAR and a D76N mutant that were used as positive controls in some of the Examples.

Since tryptophan racemase activity was detected in crude extracts from Aeromonas caviae ATCC 14486, degenerate primers were designed (based on conserved regions of known BAR homologs) to obtain the BAR gene from Aeromonas caviae ATCC 14486. Degenerate primer sequences are shown below:

Aer deg F2:

| | |
|---|---|
| 5′-GCCAGCAACGARGARGCMCGCGT-3′; | (SEQ ID NO: 541) |
| and | |
| Aer deg R1: | |
| 5′-TGGCCSTKGATCAGCACA-3′ | (SEQ ID NO: 542) |

wherein K indicates G or T, R indicates A or G, S indicates C or G, and M indicates A or C.

The above primers were used to PCR amplify a 715 bp DNA fragment from A. caviae (ATCC Accession No. 14486) genomic DNA. The following PCR protocol was used: A 50 μL reaction contained 0.5 μL template (˜100 μg of A. caviae genomic DNA), 1.6 μM of each primer, 0.3 mM each dNTP, 10 U rTth Polymerase XL (Applied Biosystems, Foster City, Calif.), 1× XL buffer, 1 mM Mg(OAc)₂ and 2.5 μL dimethyl sulfoxide. The thermocycler program used included a hot start at 94° C. for 3 minutes and 30 repetitions of the following steps: 94° C. for 30 seconds, 53° C. for 30 seconds, and 68° C. for 2 minutes. After the 30 repetitions, the sample was maintained at 68° C. for 7 minutes and then stored at 4° C. This PCR protocol produced a product of 715 bp.

The PCR product was gel purified from 0.8% TAE-agarose gel using the Qiagen gel extraction kit (Qiagen, Valencia, Calif.). The product was TOPO cloned and transformed into TOP10 cells according to manufacturer's protocol (Invitrogen, Carlsbad, Calif.). The plasmid DNA was purified from the resulting transformants using the Qiagen spin miniprep kit (Qiagen, Valencia, Calif.) and screened for the correct inserts by restriction digest with EcoR1. The sequences of plasmids appearing to have the correct insert were verified by dideoxy chain termination DNA sequencing with universal M13 forward primers.

Four libraries were constructed for each strain as per manufacturer's protocols (BD GenomeWalker™ Universal Kit, Clontech). Gene-specific primers were designed as per GenomeWalker™ manufacturer's protocols based on sequences obtained using degenerate primer sequences (see above), allowing for a few hundred homologous base pair overlap with original product. These gene-specific primers were subsequently used with GenomeWalker™ adaptor primers for PCR of upstream and downstream sequences to complete A. caviae BAR ORF.

Full-length gene sequence of the A. caviae BAR gene:

(SEQ ID NO: 543)
acgcacaaga aaacactgct cgcgaccctg atctttggcc
tgctggccgg ccaggcagtc gccgccccct atctgccgct
cgccgacgac caccgcaacg gtcaggaaca gaccgccgcc
aacgcctggc tggaagtgga tctcggcgcc ttcgagcaca
acatccagac cctgaagaat cgcctcggtg acaagggccc
gcagatctgc gccatcatga aggcggacgc ctacggtcac
ggcatcgacc tgctggtccc ttccgtggtc aaggcaggca
tcccctgcat cggcatcgcc agcaacgaag aagcacgtgt
tgcccgcgag aagggcttcg aaggtcgcct gatgcgggta
cgtgccgcca ccccggatga agtggagcag gccctgccct
acaagctgga ggagctcatc ggcagcctgg agagcgccaa
ggggatcgcc gacatcgccc agcgccatca caccaacatc
ccggtgcaca tcggcctgaa ctccgccggc atgagccgca
acggcatcga tctgcgccag gacgatgcca aggccgatgc
cctggccatg ctcaagctca aggggatcac cccggtcggc
atcatgaccc acttcccggt ggaggagaaa gaggacgtca
agctggggct ggcccagttc aagctggact accagtggct
catcgacgcc ggcaagctgg atcgcagcaa gctcaccatc
cacgccgcca actccttcgc caccctggaa gtaccggaag
cctactttga catggtgcgc ccgggcggca tcatctatgg
cgacaccatt ccctcctaca ccgagtacaa gaaggtgatg
gcgttcaaga cccaggtcgc ctccgtcaac cactacccgg
cgggcaacac cgtcggctat gaccgcacct tcaccctcaa
gcgcgactcc ctgctggcca acctgccgat gggctactcc
gacggctacc gccgcgccat gagcaacaag gcctatgtgc
tgatccatgg ccagaaggcc cccgtcgtgg gcaagacttc
catgaacacc accatggtgg acgtcaccga catcaagggg
atcaaacccg gtgacgaggt ggtcctgttc ggacgccagg
gtgatgccga ggtgaaacaa tctgatctgg aggagtacaa
cggtgccctc ttggcggaca tgtacaccgt ctggggctat
accaacccca agaagatcaa gcgctaa.

The corresponding amino acid sequence for the A. caviae native BAR:

(SEQ ID NO: 544)
MHKKTLLATL IFGLLAGQAV AAPYLPLADD HRNGQEQTAA
NAWLEVDLGA FEHNIQTLKN RLGDKGPQIC AIMKADAYGH
GIDLLVPSVV KAGIPCIGIA SNEEARVARE KGFEGRLMRV
RAATPDEVEQ ALPYKLEELI GSLESAKGIA DIAQRHHTNI
PVHIGLNSAG MSRNGIDLRQ DDAKADALAM LKLKGITPVG
IMTHFPVEEK EDVKLGLAQF KLDYQWLIDA GKLDRSKLTI
HAANSFATLE VPEAYFDMVR PGGIIYGDTI PSYTEYKKVM
AFKTQVASVN HYPAGNTVGY DRTFTLKRDS LLANLPMGYS
DGYRRAMSNK AYVLIHGQKA PVVGKTSMNT TMVDVTDIKG
IKPGDEVVLF GRQGDAEVKQ SDLEEYNGAL LADMYTVWGY
TNPKKIKR.

The following PCR primers were utilized to clone the native full-length A. caviae BAR in both tagged and C-terminally his-tagged versions:

caviae F Nde1
(SEQ ID NO: 545)
5′-GGA ACC TTC ATA TGC ACA AGA AAA CAC TGC TCG CGA
CC-3′;
caviae R BamH1 (untagged)
(SEQ ID NO: 546)
5′-GGT TCC AAG GAT CCT TAG CGC TTG ATC TTC TTG GGG
TTG-3′;
and
caviae R Xho1 (C-term tag)
(SEQ ID NO: 547)
5′-TTC CAA GGC TCG AGG CGC TTG ATC TTC TTG GGG TTG
GTA-3′.

The C-terminally tagged enzyme had comparable activity to the untagged native A. caviae BAR. When 200 μg of purified (tagged) racemase enzymes were used in a tryptophan racemase assay as described in Example 17, at 30 minutes, A. caviae BAR produced 1034 μg/mL of D-tryptophan.

### The Effect of Leader Sequences on Racemase Activity

The first 21 N-terminal amino acid residues of the A. caviae native BAR amino acid sequence above (SEQ ID NO:544) were predicted to be a signal peptide using the program Signal P 3.0 ((cbs.dtu.dk/services/SignalP/ on the World Wide Web). The following N-terminal primer was used to clone the A. caviae gene without amino acids 2-21 of the leader sequence:

A. cavMinus leader F NdeI
(SEQ ID NO: 548)
5′-CCT TGG AAC ATA TGG CCC CCT ATC TGC CGC T-3′.

The leaderless racemase, when expressed, was found to retain approximately 65% of the activity, as compared with the expression product of the full-length gene. The periplasmic and cytoplasmic protein fractions were isolated for the wild type expression products, as well as the leaderless constructs, as described in the pET System Manual (Novagen, Madison, Wis.). The majority of expressed wildtype BAR was found in the periplasm, while the leaderless BAR appeared to remain in the cytoplasm. The reduction in activity of the leaderless A. caviae BAR may be due to a change in processing and/or folding when expressed in the cytoplasm.

Effect of D76N Mutation on A. caviae BAR Activity

A D76N mutant of A. caviae BAR was made to determine if this position was critical for broad activity. Mutagenesis was done using the QuickChange-Multi site-directed mutagenesis kit (Stratagene, La Jolla, Calif.), using the C-tagged A. caviae BAR gene in pET30 as template. The following mutagenic primer was used to make a D76N change (nucleotide position 226): 5′-CGC CAT CAT GAA GGC GAA CGC CTA CGG TCA CG-3′ (SEQ ID NO:549). The site-directed mutagenesis was done as described in the manufacturer's protocol. The mutant and the wildtype enzyme were produced as described above and assayed as described in Example 17 using 200 micrograms of purified protein (prepared as described herein—purified A. caviae D76N was C-term His tagged in pET30) and approximately 7 mg/mL of L-tryptophan as substrate. At the 30 minute time point, the mutant produced 1929 micrograms per mL of D-tryptophan as compared to 1149 micrograms per mL for the wildtype enzyme. The D76N mutant also reached equilibrium at an earlier time point. The improvement in activity was unexpected.

Based on the high homology in this region for Aeromonas and Pseudomonas BAR enzymes, it might be expected that similar mutations in other broad activity racemases would also be beneficial. A benefit effect, however, was not observed when a similar mutation in SEQ ID NO:442 was generated. See Example 13.

The following racemase polypeptides had 99% identity to the BAR from A. caviae described in this example: SEQ ID NO:200, 202, 206, 142, 186 and 176. SEQ ID NO:176 had 97% identity at the amino acid level to the BAR from A. caviae described in this example. It is expected that these candidates would also have tryptophan racemase activity given the high sequence homology to an enzyme with demonstrated tryptophan racemase activity.

### Example 20

### Sequence Identity Matrix

Appendix I shows a table that describes selected characteristics of exemplary nucleic acids and polypeptides of the invention, including sequence identity comparison of the exemplary sequences to public databases. By way of example and to further aid in understanding Appendix I, the first row, labeled “SEQ ID NO:”, the numbers “1, 2” represent the exemplary polypeptide of the invention having a sequence as set forth in SEQ ID NO:2, encoded by, e.g., SEQ ID NO:1. The sequences described in Appendix I (the exemplary sequences of the invention) have been subject to a BLAST search (as described herein) against two sets of databases. The first database set is available through NCBI (National Center for Biotechnology Information). The results from searches against these databases are found in the columns entitled “NR Description”, “NR Accession Code”, “NR E-value” or “NR Organism”. “NR” refers to the Non-Redundant nucleotide database maintained by NCBI. This database is a composite of GenBank, GenBank updates, and EMBL updates. The entries in the column “NR Description” refer to the definition line in any given NCBI record, which includes a description of the sequence, such as the source organism, gene name/protein name, or some description of the function of the sequence. The entries in the column “NR Accession Code” refer to the unique identifier given to a sequence record. The entries in the column “NR E-value” refer to the Expected value (E-value), which represents the probability that an alignment score as good as the one found between the query sequence (the sequences of the invention) and that particular database sequence would be found in the same number of comparisons between random sequences as was done in the present BLAST search. The entries in the column “NR Organism” refer to the source organism of the sequence identified as the closest BLAST hit.

The second database set is collectively known as the GENESEQ™ database, which is available through Thomson Derwent (Philadelphia, Pa.). The results from searches against this database are found in the columns entitled “GENESEQ Protein Description”, “GENESEQ Protein Accession Code”, “E-value”, “GENESEQ DNA Description”, “GENESEQ DNA Accession Code” or “E-value”. The information found in these columns is comparable to the information found in the NR columns described above, except that it was derived from BLAST searches against the GENESEQ™ database instead of the NCBI databases.

In addition, this table includes the column “Predicted EC No.”. An EC number is the number assigned to a type of enzyme according to a scheme of standardized enzyme nomenclature developed by the Enzyme Commission of the Nomenclature Committee of the International Union of Biochemistry and Molecular Biology (IUBMB). The results in the “Predicted EC No.” column are determined by a BLAST search against the Kegg (Kyoto Encyclopedia of Genes and Genomes) database. If the top BLAST match has an E-value equal to or less than e-6, the EC number assigned to the top match is entered into the table.

The columns “Query DNA Length” and “Query Protein Length” refer to the number of nucleotides or the number amino acids, respectively, in the sequence of the invention that was searched or queried against either the NCBI or GENESEQ™ databases. The columns “Subject DNA Length” and “Subject Protein Length” refer to the number of nucleotides or the number amino acids, respectively, in the sequence of the top match from the BLAST searches. The results provided in these columns are from the search that returned the lower E-value, either from the NCBI databases or the GENESEQ™ database. The columns “% ID Protein” and “% ID DNA” refer to the percent sequence identity between the sequence of the invention and the sequence of the top BLAST match. The results provided in these columns are from the search that returned the lower E-value, either from the NCBI databases or the GENESEQ™ database.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of converting one or more L-amino acids to the corresponding one or more D-amino acids (or one or more D-amino acids to the corresponding one or more L-amino acids), comprising
combining one or more L-amino acids (or one or more D-amino acids) with
a) one or more nucleic acid molecules chosen from the group consisting of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, and 497, wherein said one or more nucleic acid molecules encode polypeptides having isomerase or epimerase activity;
b) a sequence variant of a), wherein said variant encodes a polypeptide having isomerase or epimerase activity;
c) a fragment of a) or b), wherein said fragment encodes a polypeptide having isomerase or epimerase activity;
d) one or more polypeptides chosen from the group consisting of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 442 D56N, 444, 446, 448, 450, 452, 454, 456, 458, 460, 462, 464, 466, 468, 470, 472, 474, 476, 478, 480, 482, 484, 486, 488, 490, 492, 494, 496, and 498, wherein said one or more polypeptides has isomerase or epimerase activity;
e) a variant of d), wherein said variant has isomerase or epimerase activity; or
f) a fragment of d) or e), wherein said fragment has isomerase or epimerase activity.

2. The method of claim 1, wherein said one or more nucleic acid molecules are chosen from the group consisting of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, and 497.

3. The method of claim 1, wherein said one or more polypeptides are chosen from the group consisting of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 386, 388, 390, 392, 394, 396, 398, 400, 402, 404, 406, 408, 410, 412, 414, 416, 418, 420, 422, 424, 426, 428, 430, 432, 434, 436, 438, 440, 442, 442 D56N, 444, 446, 448, 450, 452, 454, 456, 458, 460, 462, 464, 466, 468, 470, 472, 474, 476, 478, 480, 482, 484, 486, 488, 490, 492, 494, 496, and 498.

4. The method of claim 1, wherein the nucleic acid molecule has the sequence shown in SEQ ID NO:411.

5. The method of claim 1, wherein the polypeptide has the sequence shown in SEQ ID NO:412.

6. The method of claim 1, wherein said variant is a nucleic acid molecule that has at least 98% or at least 99% sequence identity to SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, and 497.

7. The method of claim 1, wherein said variant is a polypeptide that has at least 98% or at least 99% sequence identity to SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 442 D56N, 444, 446, 448, 450, 452, 454, 456, 458, 460, 462, 464, 466, 468, 470, 472, 474, 476, 478, 480, 482, 484, 486, 488, 490, 492, 494, 496, and 498.

8. The method of claim 1, wherein said variant is a nucleic acid that has at least 45%, at least 50%, at least 55%, at least 60%, at least 65%, at least 70%, at least 75%, at least 80%, at least 85%, at least 90%, at least 95%, or at least 99% sequence identity to SEQ ID NO:411.

9. The method of claim 1, wherein said variant is a polypeptide that has at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% sequence identity to SEQ ID NO:412.

10. The method of claim 1, wherein said variant is a mutant.

11. The method of claim 10, wherein the mutant has a mutation at the residue that aligns with residue 76 of A. caviae BAR.

12. The method of claim 1, wherein the variant is a nucleic acid molecule that has been codon optimized.

13. The method of claim 1, wherein the nucleic acid molecule is contained within an expression vector.

14. The method of claim 13, wherein the nucleic acid molecule is overexpressed.

15. The method of claim 1, wherein the polypeptide lacks a signal sequence or a prepro domain.

16. The method of claim 1, wherein the isomerase or epimerase polypeptide is immobilized on a solid support.

17. The method of claim 1, wherein the variant polypeptide is a chimeric polypeptide.

18. The method of claim 1, wherein the polypeptide fragment is a PFAM domain.

19. The method of claim 18, wherein the polypeptide fragment has the sequence shown in SEQ ID NO: 426, 440, or 462.

20. The method of claim 1, wherein the amino acid is selected from the group consisting of tryptophan, alanine and a substituted amino acid.

21. A method of converting L-tryptophan to D-tryptophan (or D-tryptophan to L-tryptophan), comprising
combining L-tryptophan (or D-tryptophan) with
a) one or more nucleic acid molecules chosen from the group consisting of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, and 497, wherein said one or more nucleic acid molecules encode polypeptides having racemase activity;
b) a variant of a), wherein said variant encodes a polypeptide having racemase activity;
c) a fragment of a) or b), wherein said fragment encodes a polypeptide having racemase activity;
d) one or more polypeptides chosen from the group consisting of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 386, 388, 390, 392, 394, 396, 398, 400, 402, 404, 406, 408, 410, 412, 414, 416, 418, 420, 422, 424, 426, 428, 430, 432, 434, 436, 438, 440, 442, 442 D56N, 444, 446, 448, 450, 452, 454, 456, 458, 460, 462, 464, 466, 468, 470, 472, 474, 476, 478, 480, 482, 484, 486, 488, 490, 492, 494, 496, and 498, wherein said one or more polypeptides has racemase activity;
e) a variant of d), wherein said variant has racemase activity; or
f) a fragment of d) or e), wherein said fragment has racemase activity.

22. The method of claim 21, wherein said one or more polypeptides are chosen from the group consisting of SEQ ID NOs:172, 178, 180, 182, 184, 140, 144, 188, 190, 112, 148, 156, 120, 162, 108, 136, 174, 138, 296, 110, 150, 192, 152, 118, 194, 154, 196, 158, 160, 116, 248, 236, 246, 252, 250, 254, 244, 274, 234, 220, 222, 226, 232, 240, 242, 258, 260, 264, 266, 286, 290, 170, 216, 288, 208, 210, 228, 230, 270, 272, 278, 280, 282, 284, 292, 198, 212, 214, 114, 218, and 204.

23. The method of claim 21, wherein said tryptophan is a substituted typtophan.

24. A method of converting L-tryptophan to D-tryptophan, comprising
combining L-tryptophan with
a) a nucleic acid molecule having the sequence shown in SEQ ID NO:411, wherein said nucleic acid molecule encodes a polypeptides having racemase activity;
b) a variant of a), wherein said variant encodes a polypeptide having racemase activity;
c) a fragment of a) or b), wherein said fragment encodes a polypeptide having racemase activity;
d) one or more polypeptides chosen from the group consisting of SEQ ID NO:411, wherein said one or more polypeptides has racemase activity;
e) a variant of d), wherein said variant has racemase activity; or
f) a fragment of d) or e), wherein said fragment has racemase activity.

25. The method of claim 24, wherein said tryptophan is a substituted tryptophan.

26. The method of claim 25, wherein said substituted tryptophan is selected from the group consisting of a chlorinated tryptophan, a halogenated tryptophan and 6-chloro-D-tryptophan.

27. A method of making monatin, comprising:
combining L-tryptophan with
a) one or more nucleic acid molecules chosen from the group consisting of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, and 497, wherein said one or more nucleic acid molecules encode polypeptides having racemase activity;
b) a variant of a), wherein said variant encodes a polypeptide having racemase activity;
c) a fragment of a) or b), wherein said fragment encodes a polypeptide having racemase activity;
d) one or more polypeptides chosen from the group consisting of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 386, 388, 390, 392, 394, 396, 398, 400, 402, 404, 406, 408, 410, 412, 414, 416, 418, 420, 422, 424, 426, 428, 430, 432, 434, 436, 438, 440, 442, 442 D56N, 444, 446, 448, 450, 452, 454, 456, 458, 460, 462, 464, 466, 468, 470, 472, 474, 476, 478, 480, 482, 484, 486, 488, 490, 492, 494, 496, and 498, wherein said one or more polypeptides has racemase activity;
e) a variant of d), wherein said variant has racemase activity; or
f) a fragment of d) or e), wherein said fragment has racemase activity.

28. The method of claim 27, further comprising adding one or more polypeptides having synthase/lyase (EC 4.1.3.- or EC 4.1.2.-) activity or a nucleic acid encoding such a polypeptide and/or one or more polypeptides having D-aminotransferase activity or a nucleic acid encoding such a polypeptide.

29. The method of claim 27, wherein said monatin is predominantly R,R monatin.

30. The method of claim 27, wherein said nucleic acid has the sequence shown in SEQ ID NO:411 and said polypeptide has the sequence shown in SEQ ID NO:412.
